(19) United States
(12) Patent Application Publication
(10) Pub. No.: US 2009/0054649 A1
(43) Pub. Date: Feb. 26, 2009

(54) Bridged Organosilane and Production Method Thereof

(75) Inventors: Toyoshi Shimada, Soraku-gun (JP); Yasutomo Goto, Owariasahi-shi (JP); Shinji Inagaki, Nagoya-shi (JP); Norihiro Mizoshita, Nagoya-shi (JP); Masamichi Ikai, Kitanagoya-shi (JP)

Correspondence Address: OLIFF & BERRIDGE, PLC, P.O. BOX 320850, ALEXANDRIA, VA, 22320-4850, US

(73) Assignees: KABUSHIKI KAISHA TOYOTA CHUO KENKYUSHO, AICHI (JP); TOYOSHI SHIMADA, KYOTO (JP)

(21) Appl. No.: 11/992,139

(22) PCT Filed: Sep. 21, 2006
(86) PCT No.: PCT/JP2006/318712

§ 371 Date: Mar. 17, 2008

(30) Foreign Application Priority Data
Sep. 22, 2005 (JP) 2005-276924
Mar. 9, 2006 (JP) 2006-064828

(51) Int. Cl. C07D 221/08; C07F 7/08; C07D 209/58; C07F 7/10

(52) U.S. Cl. 546/14; 556/432; 548/406; 556/413

(57) ABSTRACT

Provided is a bridged organosilane, which has a large complex organic group, and which is useful in the synthesis of a mesoporous silica and a light-emitting material, and a production method of the bridged organosilane. The bridged organosilane is expressed by the following general formula (1):

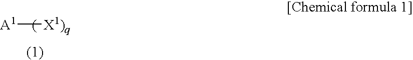

[in the formula (1), q represents an integer in a range from 2 to 4, X¹— represents a substituent selected from the group consisting of substituents expressed by the following general formulae (2) to (5):

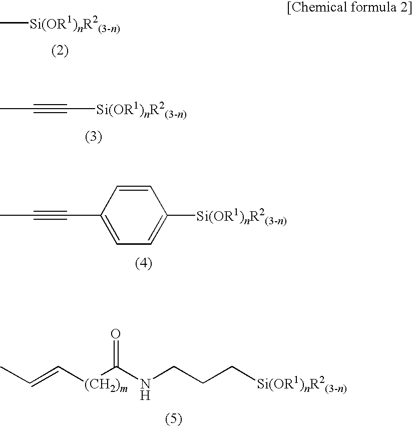

(in the formulae (2) to (5), R¹ represents alkyl group having 1 to 5 carbon atoms, R² represents an allyl group, and n represents an integer in a range from 0 to 3, and m represents an integer in a range from 0 to 6), and A¹ represents an organic group expressed by, for example, the following general formula (6):

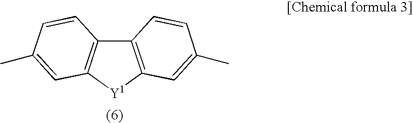

(in the formula (6), Y¹< represents a substituent expressed by, for example, O═C<)].

### TECHNICAL FIELD

The present invention relates to a bridged organosilane and a production method thereof.

### BACKGROUND OF THE INVENTION

Studies have been conducted on various bridged organosilanes. For example, a bridged organosilane expressed by the following formula:

(R′O)₃—Si—R—Si—(OR′)₃

[in the formula, R represents a phenyl group, a biphenyl group, a terphenyl group, or an anthracene group, and R′ represents a methyl group or an ethyl group] and a production method thereof have been reported (K. J. Shea et. al., J. American. Chemical. Society. 1992, vol. 114, No. 17, pp. 6700-6709).

However, in a conventional method which has been reported for the synthesis of a bridged organosilane, as R in the formula becomes more complex and larger, the synthesis becomes more difficult to achieve. Accordingly, it is still impossible to obtain a bridged organosilane having a complex organic group where R is fluorene, quaterphenyl, or the like.

On the other hand, in such a conventional method for the synthesis of a bridged organosilane, it is possible to obtain a bridged organosilane having anthracene in the position of R in the formula, and having silanes bound at the 9- and 10-positions of the anthracene. Nonetheless, when the bridged silane is used for the synthesis of a mesoporous material, a steric hindrance occurs. As a result, there is a problem that it is difficult to synthesize a mesoporous material.

### DISCLOSURE OF THE INVENTION

The present invention has been made in consideration of the problems in the background art. An object of the present invention is to provide a bridged organosilane, which has a large complex organic group, and which is useful for the synthesis of a mesoporous silica and light-emitting material, and to provide a production method of the bridged organosilane.

The present inventors have devoted themselves to keen studies so as to achieve the above object. As a result, the present inventors have discovered that the reaction between a specific organic compound and a specific silane compound leads to the achievement of the above object. Thus, the present inventors have completed the present invention.

Specifically, the bridged organosilane according to the present invention is expressed by the following general formula (1):

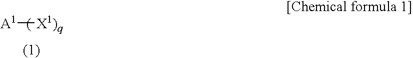

[in the formula (1), q represents an integer in a range from 2 to 4, X¹— represents a substituent selected from the group consisting of substituents expressed by the following general formulae (2) to (5):

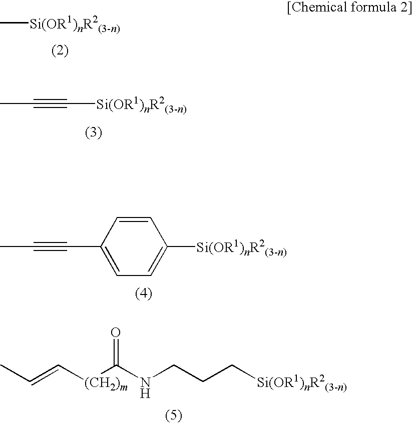

(in the formulae (2) to (5), R¹ represents any one of alkyl groups having 1 to 5 carbon atoms, R² represents an allyl group, n represents an integer in a range from 0 to 3, and m represents an integer in a range from 0 to 6), and A¹ represents one organic group selected from the group consisting of organic groups expressed by the following general formula (6):

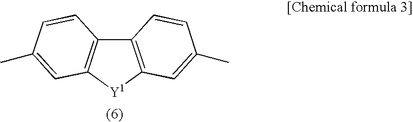

{in the formula (6), Y¹< represents a substituent selected from the group consisting of substituents expressed by the following general formulae (7) to (12):

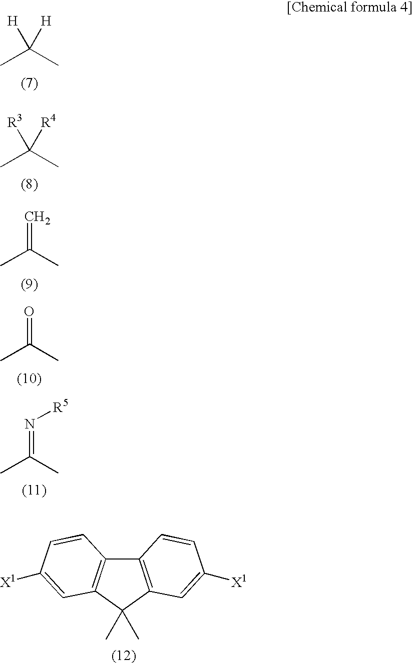

(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (12), X¹— represents a substituent selected from the group consisting of substituents expressed by the formulae (2) to (5))},
organic groups expressed by the following general formulae (13) and (14):

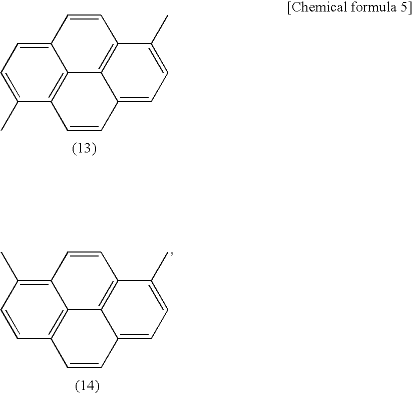

organic group expressed by the following general formulae (15) to (17):

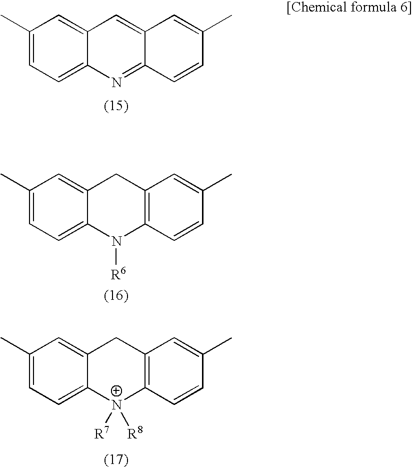

(in the formula (16), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (17) R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms),
an organic group expressed by the following general formula (18):

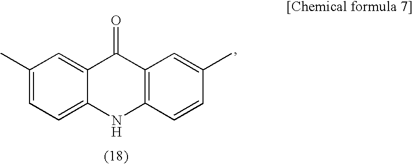

an organic group expressed by the following general formula (19):

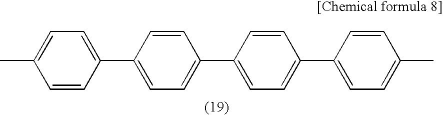

organic groups expressed by the following general formulae (20) and (21):

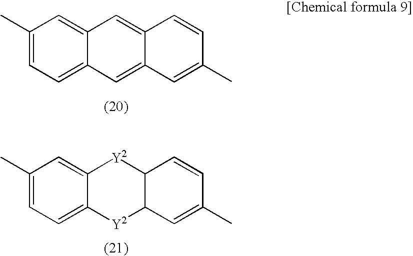

{in the formula (21), Y²< represents a substituent expressed by anyone of the following general formulae (10) and (11):

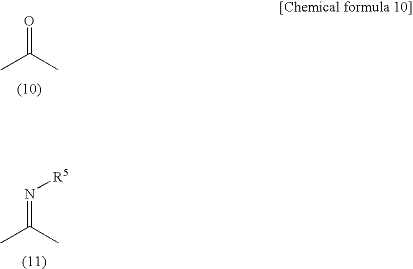

(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms)},
organic groups expressed by the following general formulae (22) and (23):

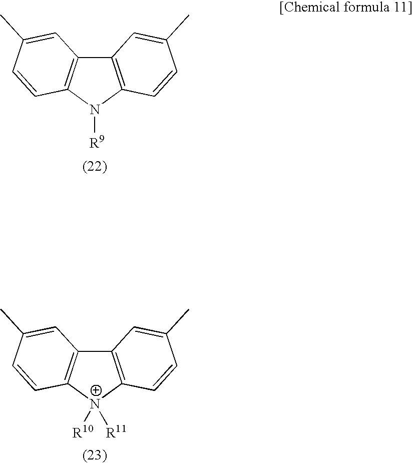

(in the formula (22), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (23) R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formula (24):

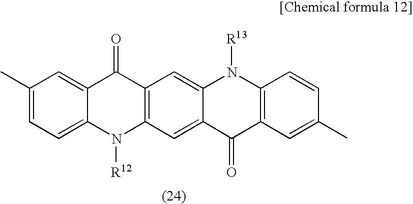

(in the formula (24), R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formulae (25) and (26):

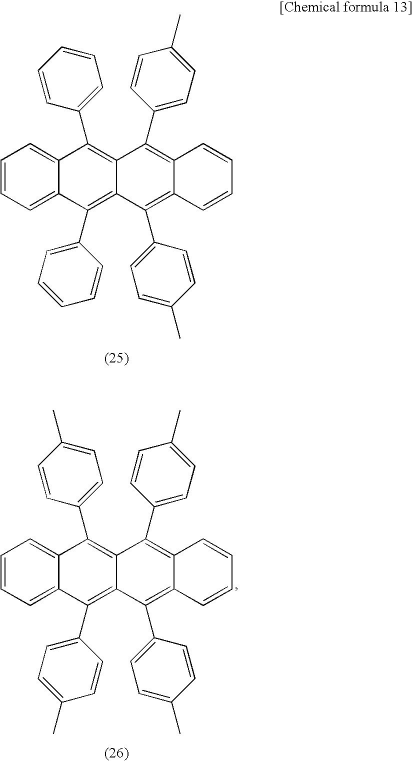

organic groups expressed by the following general formula (27):

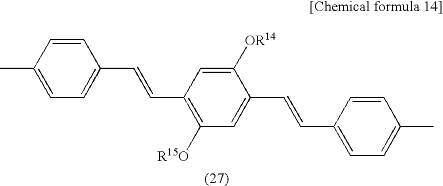

(in the formula (27), R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms), and an organic group expressed by the following general formula (28):

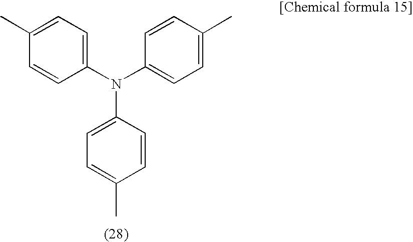

As the bridged organosilane of the present invention, preferable is a bridged organosilane (i) which is a fluorene-silane compound expressed by the following general formula (29):

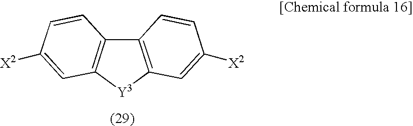

[in the formula (29), X²— represents a substituent selected from the group consisting of substituents expressed by the following general formulae (2) to (4):

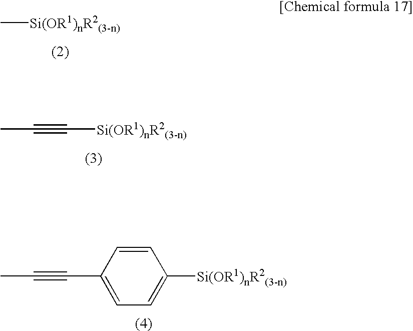

(in the formulae (2) to (4), R¹ represents any one of alkyl groups having 1 to 5 carbon atoms, R² represents an allyl group, and n represents an integer in a range from 0 to 3), and Y³< represents a substituent selected from the group consisting of substituents expressed by the following general formulae (7) to (11) and (30):

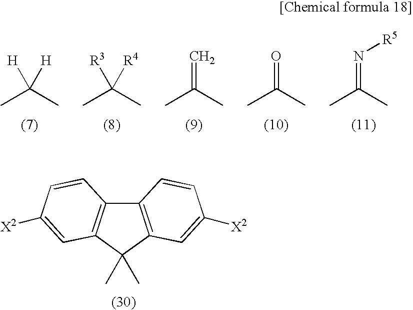

(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (30), X²— represents a substituent selected from the group consisting of substituents expressed by the formulae (2) to (4))].

Additionally, as the bridged organosilane of the present invention, preferable is a bridged organosilane (ii) which is a pyrene-silane compound expressed by any one of the following general formula (31) and (32):

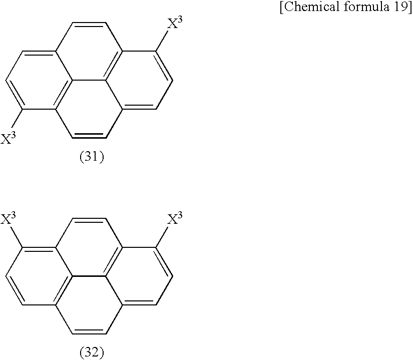

[in the formulae (31) and (32), X³— represents a substituent expressed by the following general formula (2):

### [Chemical Formula 20]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3)].

Moreover, as the bridged organosilane of the present invention, preferable is a bridged organosilane (iii) which is an acridine-silane compound expressed by any one of the following general formula (33), (34) and (35):

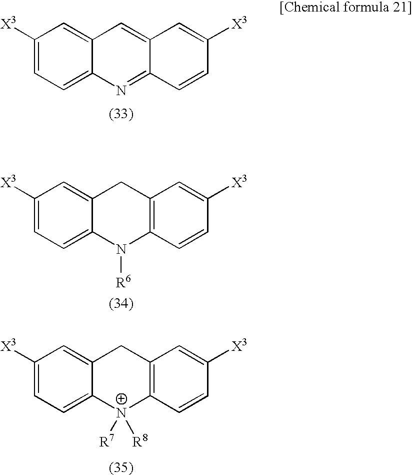

[in the formulae (33) to (35), X³— represents a substituent expressed by the following general formula (2):

### [Chemical Formula 22]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3);
in the formula (34), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (35) R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms].

Furthermore, as the bridged organosilane of the present invention, preferable is a bridged organosilane (iv) which is an acridone-silane compound expressed by the following general formula (36):

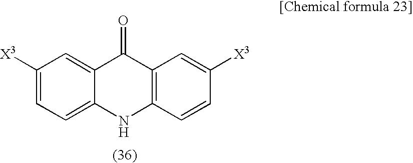

[in the formula (36), X³— represents a substituent expressed by the following general formula (2):

### [Chemical Formula 24]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3)].

In addition, as the bridged organosilane of the present invention, preferable is a bridged organosilane (v) which is a quaterphenyl-silane compound expressed by the following general formula (37):

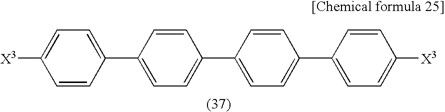

[in the formula (37), X³— represents a substituent expressed by the following general formula (2):

### [Chemical Formula 26]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3)].

Moreover, as the bridged organosilane of the present invention, preferable is abridged organosilane (vi) which is an anthracene-silane compound, an anthraquinone-silane compound or an anthraquinonediimine-silane compound, expressed by any one of the following general formula (38) and (39):

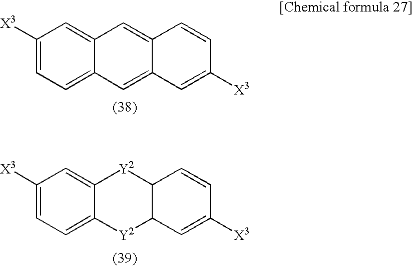

[in the formulae (38) and (39), X³— represents a substituent expressed by the following general formula (2):

### [Chemical Formula 28]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3); and
in the formula (39), Y²< represents a substituent expressed by any one of the following general formulae (10) and (11):

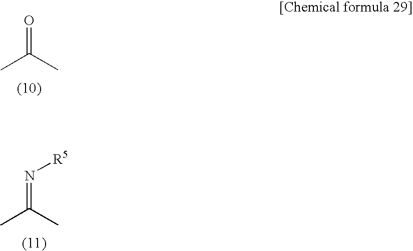

(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms)].

Furthermore, as the bridged organosilane of the present invention, preferable is a bridged organosilane (vii) which is a carbazole-silane compound expressed by any one of the following general formula (40) and (41):

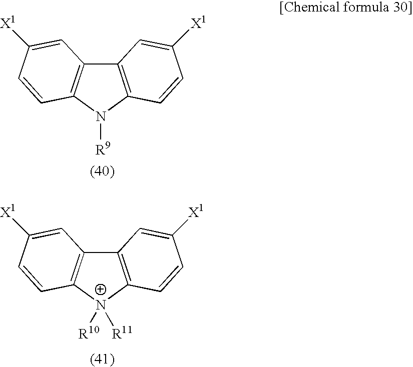

[in the formulae (40) and (41), X¹— represents a substituent selected from the group consisting of substituents expressed by the following general formulae (2) to (5):

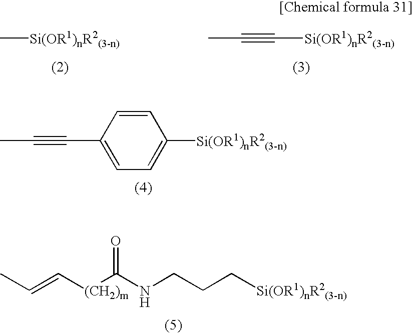

(in the formulae (2) to (5), R¹ represents any one of alkyl groups having 1 to 5 carbon atoms, R² represents an allyl group, n represents an integer in a range from 0 to 3, and m represents an integer in a range from 0 to 6); in the formula (40), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (41), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms].

Additionally, as the bridged organosilane of the present invention, preferable is a bridged organosilane (viii) which is a quinacridone-silane compound expressed by the following general formula (42):

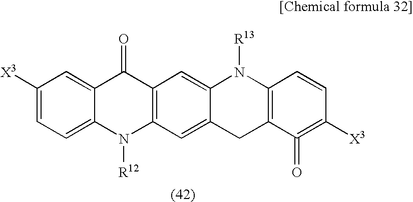

[in the formula (42), X³— represents a substituent expressed by the following general formula (2):

### [Chemical Formula 33]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3), and R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms].

Moreover, as the bridged organosilane of the present invention, preferable is abridged organosilane (ix) which is a rubrene-silane compound expressed by the following general formula (43) or (44):

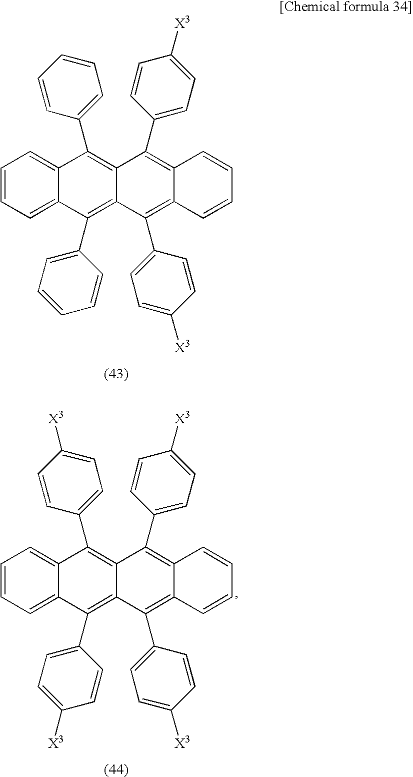

[in the formulae (43) and (44), X³— represents a substituent expressed by the following general formula (2):

### [Chemical Formula 35]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3)].

Furthermore, as the bridged organosilane of the present invention, preferable is a bridged organosilane (x) which is a 1,4-alkyloxy-2,5-phenylethenylbenzene-silane compound expressed by the following general formula (45):

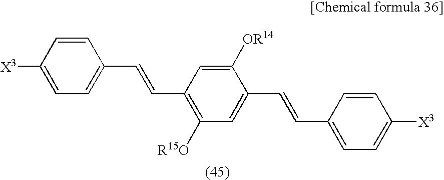

[in the formula (45), X³— represents a substituent expressed by the following general formula (2):

### [Chemical Formula 37]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3), and R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms].

Still furthermore, as the bridged organosilane of the present invention, preferable is a bridged organosilane (xi) which is a triphenylamine-silane compound expressed by the following general formula (46):

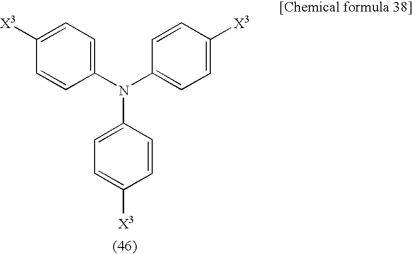

[in the formula (46), X³— represents a substituent expressed by the following general formula (2):

### [Chemical Formula 39]

—Si(OR¹)ₙR²₍₃₋ₙ₎ (2)

(in the formula (2), R¹ represents any one of alkyl groups having 1 to 5 carbon atoms, R² represents an allyl group, and n represents an integer in a range from 0 to 3)].

In a bridged organosilane production method of the present invention, the bridged organosilane of the present invention is obtained by causing a compound expressed by the following general formula (47):

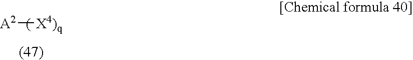

to react with a silane compound expressed by the following general formula (54):

### [Chemical Formula 55]

H—Si(OR¹)₃ (54)

In the formula (47), q represents an integer in a range from 2 to 4, X⁴— represents a substituent selected from the group consisting of substituents expressed by the following general formulae (48) to (51):

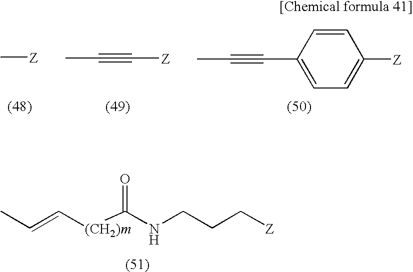

(in the formulae (48) to (51), Z represents any one of halogen atoms, a hydroxy group, and a fluoromethanesulfonate group, and m represents an integer in a range from 0 to 6), and A² represents one organic group selected from the group consisting of organic groups expressed by the following general formula (52):

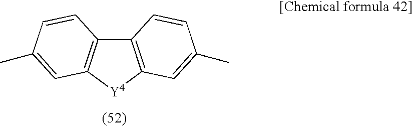

{in the formula (52), Y⁴< represents a substituent selected from the group consisting of substituents expressed by the following general formulae (7) to (11) and (53):

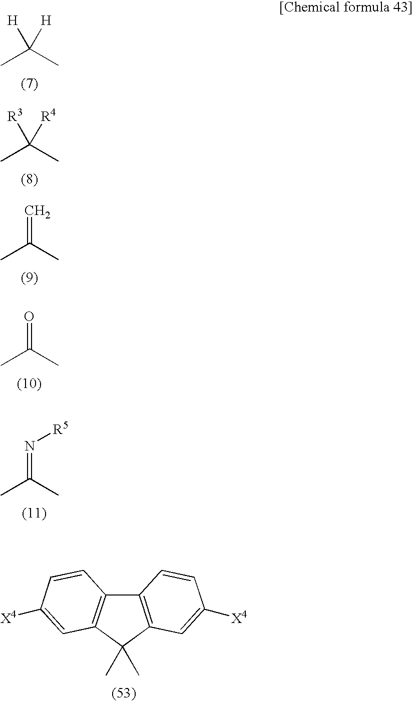

(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (53), X⁴— represents a substituent selected from the group consisting of substituents expressed by the formulae (48) to (51))},
organic groups expressed by the following general formulae (13) and (14):

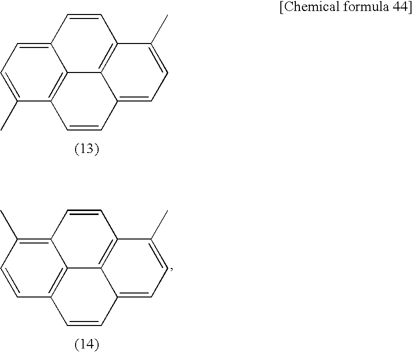

organic groups expressed by the following general formulae (15) to (17):

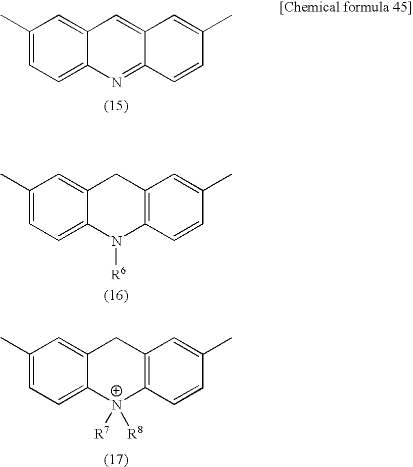

(in the formula (16), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (17), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms),
an organic group expressed by the following general formula (18):

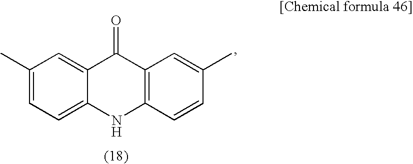

an organic group expressed by the following general formula (19):

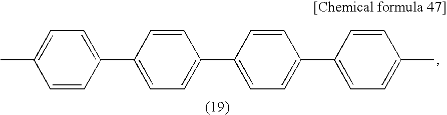

organic groups expressed by the following general formulae (20) and (21):

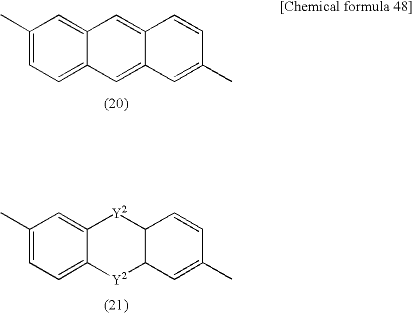

{in the formula (21), Y²< represents a substituent expressed by any one of the following general formulae (10) and (11):

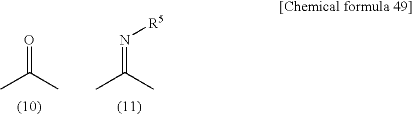

(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms)},
organic groups expressed by the following general formulae (22) and (23):

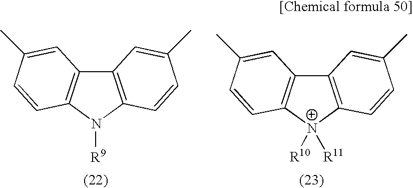

(in the formula (22), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (23), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formula (24):

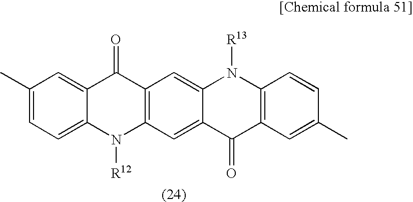

(in the formula (24), R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formulae (25) and (26):

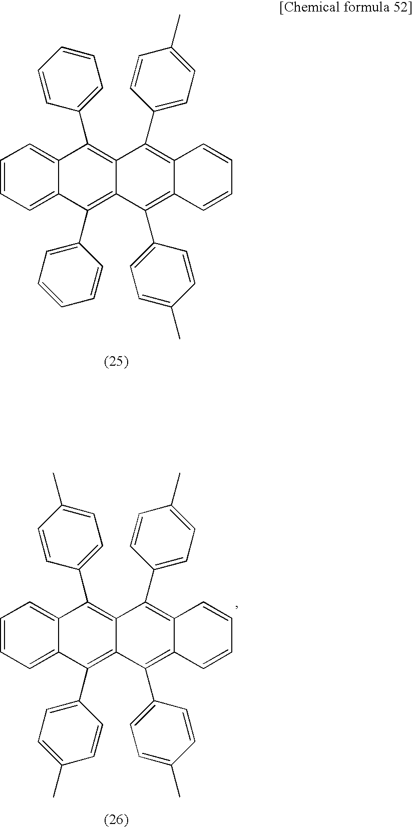

organic groups expressed by the following general formula (27):

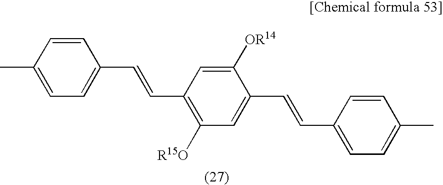

(in the formula (27), R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms), and an organic group expressed by the following general formula (28):

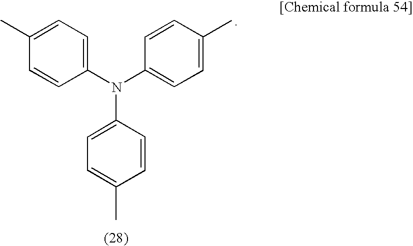

In the general formula (54), R¹ represents any one of alkyl groups having 1 to 5 carbon atoms.

Additionally, in the preferable bridged organosilane production method of the present invention, the bridged organosilane (i), which is the fluorene-silane compound, is obtained by causing a fluorene compound expressed by the following general formula (55):

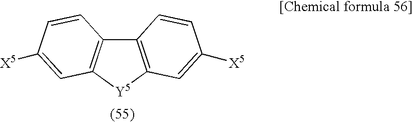

to react with a silane compound expressed by the following general formula (54):

### [Chemical Formula 59]

H—Si(OR¹)₃ (54)

In the formula (55), X⁵— represents a substituent selected from the group consisting of substituents expressed by the following general formulae (48) to (50):

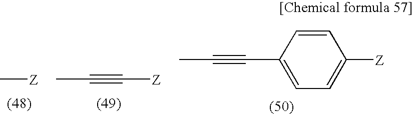

(in the formulae (48) to (50), Z represents any one of a halogen atom, a hydroxy group, and a fluoromethanesulfonate group), and Y⁵< represents a substituent selected from the group consisting of substituents expressed by the following general formulae (7) to (11) and (56):

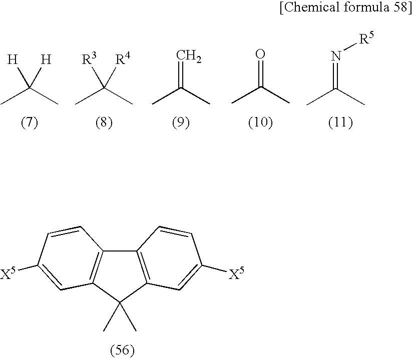

(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (56), X⁵— represents a substituent selected from the group consisting of substituents expressed by the formulae (48) to (50)).

In the formula (54), R¹ represents any one of alkyl groups having 1 to 5 carbon atoms).

Moreover, in the bridged organosilane production method of the present invention, the bridged organosilane (ii), which is the pyrene-silane compound, is obtained by causing a pyrene compound expressed by any one of the following general formulae (57) and (58):

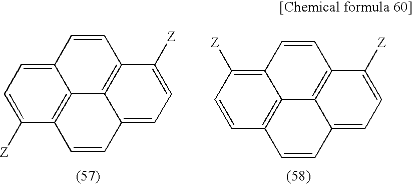

(in the formulae (57) and (58), Z represents any one of a halogen atom, a hydroxy group, and a fluoromethanesulfonate group)
to react with a silane compound expressed by the following general formula (54):

### [Chemical Formula 61]

H—Si(OR¹)₃ (54)

(in the formula (54), R¹ represents any one of alkyl groups having 1 to 5 carbon atoms).

Furthermore, in the bridged organosilane production method of the present invention, the bridged organosilane (iii), which is the acridine-silane compound, is obtained by causing an acridine compound expressed by the following general formulae (59), (60) and (61):

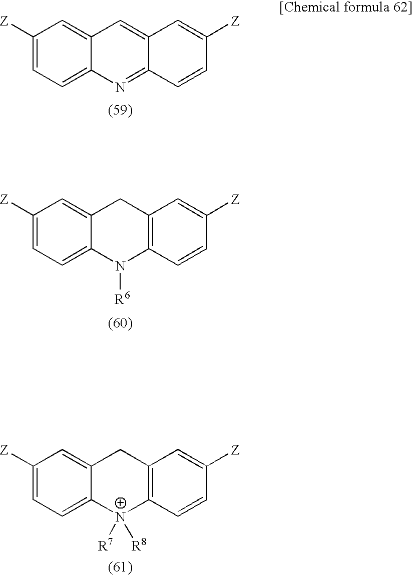

(in the formulae (59) to (61), Z represents any one of a halogen atom, a hydroxy group, and a fluoromethanesulfonate group; in the formula (60), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (61), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms)
to react with a silane compound expressed by the following general formula (54):

### [Chemical Formula 63]

H—Si(OR¹)₃ (54)

(in the formula (54), R¹ represents any one of alkyl groups having 1 to 5 carbon atoms).

Additionally, in the bridged organosilane production method of the present invention, the bridged organosilane (iv), which is the acridone-silane compound, is obtained by causing an acridone compound expressed by the following general formula (62):

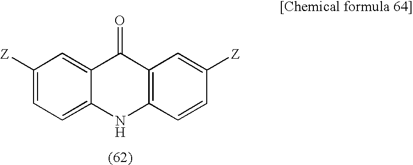

(in the formula (62), Z represents any one of a halogen atom, a hydroxy group, and a fluoromethanesulfonate group)
to react with a silane compound expressed by the following general formula (54):

### [Chemical Formula 65]

H—Si(OR¹)₃ (54)

(in the formula (54), R¹ represents any one of alkyl groups having 1 to 5 carbon atoms).

Moreover, in the bridged organosilane production method of the present invention, the bridged organosilane (v), which is the quaterphenyl-silane compound, is obtained by causing a quaterphenyl compound expressed by the following general formula (63):

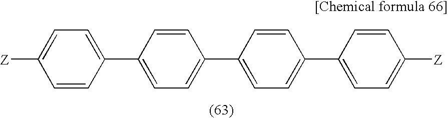

(in the formula (63), Z represents any one of a halogen atom, a hydroxy group, and a fluoromethanesulfonate group)
to react with a silane compound expressed by the following general formula (54):

### [Chemical Formula 67]

H—Si(OR¹)₃ (54)

(in the formula (54), R¹ represents any one of alkyl groups having 1 to 5 carbon atoms).

Furthermore, in the bridged organosilane production method of the present invention, the bridged organosilane (vi), which is the anthracene-silane compound, is obtained by causing an anthracene compound expressed by the following general formula (64):

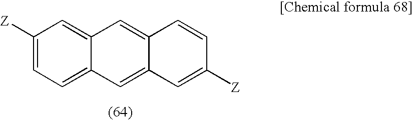

[in the formula (64), Z represents any one of a halogen atom, a hydroxy group, and a fluoromethanesulfonate group]
to react with a silane compound expressed by the following general formula (54):

### [Chemical Formula 69]

H—Si(OR¹)₃ (54)

(in the formula (54), R¹ represents any one of alkyl groups having 1 to 5 carbon atoms).

Additionally, in the bridged organosilane production method of the present invention, the bridged organosilane (vii), which is the carbazole-silane compound, is obtained by causing a carbazole compound expressed by any one of the following general formulae (65) and (66):

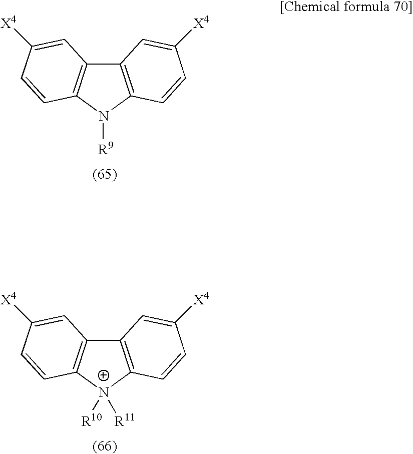

[in the formulae (65) and (66), X⁴— represents a substituent selected from the group consisting of substituents expressed by the following general formulae (48) to (51):

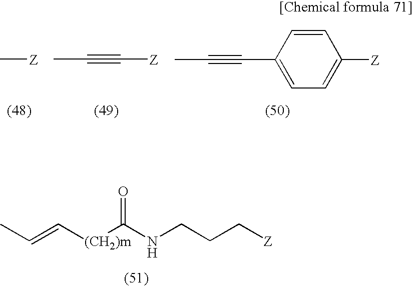

(in the formulae (48) to (51), Z represents any one of a halogen atom, a hydroxy group, and a fluoromethanesulfonate group, and m represents an integer in a range from 0 to 6); in the formula (65), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (66), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms]
to react with a silane compound expressed by the following general formula (54):

### [Chemical Formula 72]

H—Si(OR¹)₃ (54)

(in the formula (54), R¹ represents any one of alkyl groups having 1 to 5 carbon atoms).

Moreover, in the bridged organosilane production method of the present invention, the bridged organosilane (viii), which is the quinacridone-silane compound, is obtained by causing a quinacridone compound expressed by the following general formula (67):

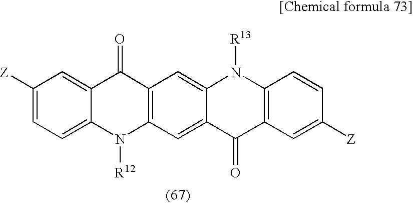

[in the formula (67), Z represents any one of a halogen atom, a hydroxy group, and a fluoromethanesulfonate group]
to react with a silane compound expressed by the following general formula (54):

### [Chemical Formula 74]

H—Si(OR¹)₃ (54)

(in the formula (54), R¹ represents any one of alkyl groups having 1 to 5 carbon atoms).

Furthermore, in the bridged organosilane production method of the present invention, the bridged organosilane (ix), which is the rubrene-silane compound, is obtained by causing a rubrene compound expressed by any one of the following general formulae (68) and (69):

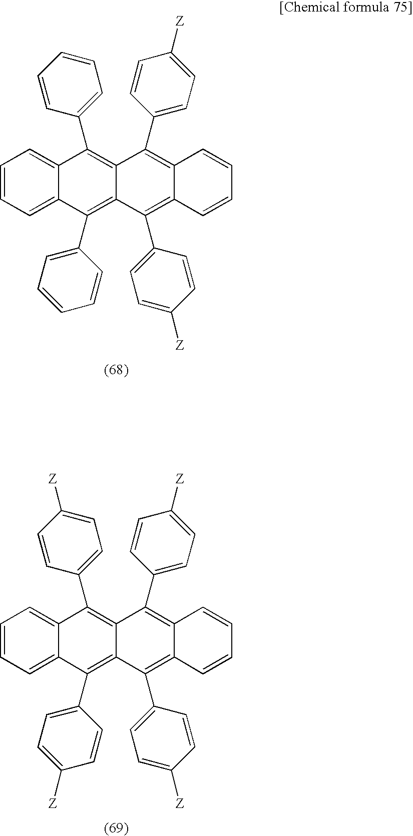

[in the formulae (68) and (69), Z represents any one of a halogen atom, a hydroxy group, and a fluoromethanesulfonate group]
to react with a silane compound expressed by the following general formula (54):

### [Chemical Formula 76]

H—Si(OR¹)₃ (54)

(in the formula (54), R¹ represents any one of alkyl groups having 1 to 5 carbon atoms).

Additionally, in the bridged organosilane production method of the present invention, the bridged organosilane (x), which is a 1,4-alkyloxy-2,5-phenylethenylbenzene-silane compound, is obtained by causing the 1,4-alkyloxy-2,5-phenylethenylbenzene compound expressed by the following general formula (70):

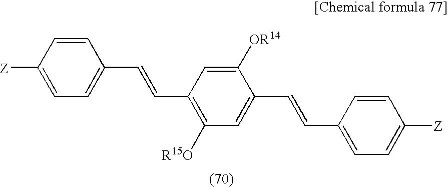

[in the formula (70), Z represents any one of a halogen atom, a hydroxy group, and a fluoromethanesulfonate group]
to react with a silane compound expressed by the following general formula (54):

### [Chemical Formula 78]

H—Si(OR¹)₃ (54)

(in the formula (54), R¹ represents any one of alkyl groups having 1 to 5 carbon atoms).

Moreover, in the bridged organosilane production method of the present invention, the bridged organosilane (xi), which is the triphenylamine-silane compound, is obtained by causing a triphenylamine compound expressed by the following general formula (71):

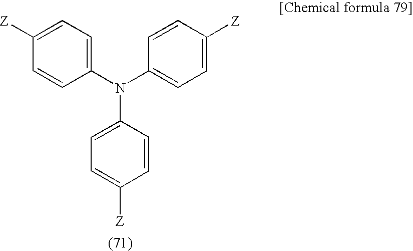

[in the formula (71), Z represents any one of a halogen atom, a hydroxy group, and a fluoromethanesulfonate group]
to react with a silane compound expressed by the following general formula (54):

### [Chemical Formula 80]

H—Si(OR¹)₃ (54)

(in the formula (54), R¹ represents any one of alkyl groups having 1 to 5 carbon atoms).

According to the present invention, it is possible to provide a bridged organosilane, which has a large complex organic group, and which is useful in the synthesis of mesoporous silica and a light-emitting material, and to provide a production method of the bridged organosilane.

### BRIEF DESCRIPTION OF THE DRAWINGS

FIG. 1 is a graph of a ¹H NMR measurement on a fluorene-silane compound obtained in Example 1.

FIG. 2 is a graph of ¹H NMR measurement on the fluorene-silane compound obtained in Example 1.

FIG. 3 is a graph of a ¹H NMR measurement on the fluorene-silane compound obtained in Example 1.

FIG. 4 is a graph showing a UV spectrum of the fluorene-silane compound obtained in Example 1.

FIG. 5 is a graph of a ¹H NMR measurement on a pyrene-silane compound obtained in Example 2.

FIG. 6 is a graph of a ¹H NMR measurement on the pyrene-silane compound obtained in Example 2.

FIG. 7 is a graph of a ¹H NMR measurement on the pyrene-silane compound obtained in Example 2.

FIG. 8 is a graph of a ¹H NMR measurement on the pyrene-silane compound obtained in Example 2.

FIG. 9 is a graph showing a UV spectrum of the pyrene-silane compound obtained in Example 2.

FIG. 10 is a graph showing a UV spectrum of 2,7-dibromoacridine obtained in Example 3.

FIG. 11 is a graph showing a UV spectrum of the 2,7-dibromoacridine obtained in Example 3.

FIG. 12 is a graph of a ¹H NMR measurement on an acridine-silane compound obtained in Example 3.

FIG. 13 is a graph of a ¹H NMR measurement on the acridine-silane compound obtained in Example 3.

FIG. 14 is a graph of a ¹H NMR measurement on the acridine-silane compound obtained in Example 3.

FIG. 15 is a graph showing a UV spectrum of the acridine-silane compound obtained in Example 3.

FIG. 16 is a graph showing a UV spectrum of acridone.

FIG. 17 is a graph showing a UV spectrum of 2,7-dibromoacridone obtained in Example 4.

FIG. 18 is a graph of a ¹H NMR measurement on an acridone-silane compound obtained in Example 4.

FIG. 19 is a graph of a ¹H NMR measurement on the acridone-silane compound obtained in Example 4.

FIG. 20 is a graph showing a UV spectrum of the acridone-silane compound obtained in Example 4.

FIG. 21 is a graph of a ¹³C NMR measurement on a quaterphenyl-silane compound obtained in Example 5.

FIG. 22 is a graph of a ¹H NMR measurement on the quaterphenyl-silane compound obtained in Example 5.

FIG. 23 is a graph of a ¹H NMR measurement on the quaterphenyl-silane compound obtained in Example 5.

FIG. 24 is a graph of a ¹H NMR measurement on the quaterphenyl-silane compound obtained in Example 5.

FIG. 25 is a graph showing a UV spectrum of the quaterphenyl-silane compound obtained in Example 5.

FIG. 26 is a graph of a ¹H NMR measurement on 2,6-dihydroxyanthracene obtained in Example 6.

FIG. 27 is a graph of a ¹H NMR measurement on the 2,6-dihydroxyanthracene obtained in Example 6.

FIG. 28 is a graph of a ¹H NMR measurement on an anthracene compound obtained in Example 6.

FIG. 29 is a graph of a ¹H NMR measurement on the anthracene compound obtained in Example 6.

FIG. 30 is a graph showing a UV spectrum of an anthracene-silane compound obtained in Example 6.

FIG. 31 is a graph of a ¹H NMR measurement on the anthracene-silane compound obtained in Example 6.

FIG. 32 is a graph of a ¹H NMR measurement on the anthracene-silane compound obtained in Example 6.

FIG. 33 is a graph showing an X-ray diffraction pattern of a Flu-HMM-s-film obtained in Example 7.

FIG. 34 is a graph showing a fluorescence spectrum and an excitation spectrum of the Flu-HMM-s-film obtained in Example 7.

FIG. 35 is a graph showing a UV spectrum of the Flu-HMM-s-film obtained in Example 7.

FIG. 36 is a graph showing an X-ray diffraction pattern of a Flu-HMM-powder obtained in Example 8.

FIG. 37 is a graph showing a fluorescence spectrum and an excitation spectrum obtained in Example 8.

FIG. 38 is a graph showing an X-ray diffraction pattern of a Pyr-HMMc-s-film obtained in Example 9.

FIG. 39 is a graph showing a fluorescence spectrum (solid line, excitation wavelength: 350 nm) and an excitation spectrum (dashed line, measured wavelength: 450 nm) of the Pyr-HMMc-s-film obtained in Example 9.

FIG. 40 is a graph showing a UV spectrum of the Pyr-HMMc-s-film obtained in Example 9.

FIG. 41 is a graph showing a fluorescence spectrum (solid line, excitation wavelength: 350 nm) and an excitation spectrum (dashed line, measured wavelength: 450 nm) of a Pyr-acid-film obtained in Example 10.

FIG. 42 is a graph showing a UV spectrum of the Pyr-acid-film obtained in Example 10.

FIG. 43 is a graph showing a fluorescence spectrum and an excitation spectrum of a Flu-HMM-powder obtained in Example 11.

FIG. 44 is a graph showing a fluorescence spectrum and an excitation spectrum of the Pyr-HMM-s-film obtained in Example 11.

FIG. 45 is a graph showing a UV spectrum of the Pyr-HMM-s-film obtained in Example 11.

FIG. 46 is a graph showing an X-ray diffraction pattern of a Pyr-Acid-powder obtained in Example 12.

FIG. 47 is a graph showing a fluorescence spectrum and an excitation spectrum of the Pyr-Acid-powder obtained in Example 12.

FIG. 48 is a graph showing an X-ray diffraction pattern of an Ant-Acid-powder obtained in Example 13.

FIG. 49 is a graph showing a fluorescence spectrum and an excitation spectrum of the Ant-Acid-powder obtained in Example 13.

FIG. 50 is a graph showing an X-ray diffraction pattern of an Ant-HMM-s-film obtained in Example 14.

FIG. 51 is a graph showing a fluorescence spectrum and an excitation spectrum of the Ant-HMM-s-film obtained in Example 14.

FIG. 52 is a graph showing a UV spectrum of the Ant-HMM-s-film obtained in Example 14.

FIG. 53 is a graph showing a fluorescence spectrum and an excitation spectrum of an Acr-HMM-s-film obtained in Example 15.

FIG. 54 is a graph showing an X-ray diffraction pattern of an Acr-HMM-powder obtained in Example 16.

FIG. 55 is a graph showing a fluorescence spectrum and an excitation spectrum of the Acr-HMM-powder obtained in Example 16.

FIG. 56 is a graph showing an X-ray diffraction pattern of a Qua-HMM-powder obtained in Example 17.

FIG. 57 is a graph showing a fluorescence spectrum and an excitation spectrum of the Qua-HMM-powder obtained in Example 17.

FIG. 58 is a graph showing an X-ray diffraction pattern of an Acd-HMM-s-film obtained in Example 18.

FIG. 59 is a graph showing a fluorescence spectrum and an excitation spectrum of the Acd-HMM-s-film obtained in Example 18.

FIG. 60 is a graph showing a UV spectrum of the Acd-HMM-s-film obtained in Example 18.

FIG. 61 is a graph showing an X-ray diffraction pattern of an Acd-HMM-powder obtained in Example 19.

FIG. 62 is a graph showing a fluorescence spectrum and an excitation spectrum of the Acd-HMM-powder obtained in Example 19.

FIG. 63 is a graph of a ¹³C NMR measurement on 3,6-diiodocarbazole obtained in Example 20.

FIG. 64 is a graph of a ¹¹H NMR measurement on the 3,6-diiodocarbazole obtained in Example 20.

FIG. 65 is a graph of a ¹¹H NMR measurement on the 3,6-diiodocarbazol obtained in Example 20.

FIG. 66 is a graph of a ¹³C NMR measurement on a carbazole-silane compound obtained in Example 20.

FIG. 67 is a graph of a ¹H NMR measurement on the carbazole-silane compound obtained in Example 20.

FIG. 68 is a graph of a ¹H NMR measurement on the carbazole-silane compound obtained in Example 20.

FIG. 69 is a graph of a ¹³C NMR measurement on 3,6-diiodo-9-methylcarbazole obtained in Example 21.

FIG. 70 is a graph of a ¹H NMR measurement on the 3,6-diiodo-9-methylcarbazole obtained in Example 21.

FIG. 71 is a graph of a ¹H NMR measurement on the 3,6-diiodo-9-methylcarbazole obtained in Example 21.

FIG. 72 is a graph of a ¹³C NMR measurement on a carbazole-silane compound obtained in Example 21.

FIG. 73 is a graph of a ¹H NMR measurement on the carbazole-silane compound obtained in Example 21.

FIG. 74 is a graph of a ¹H NMR measurement on the carbazole-silane compound obtained in Example 21.

FIG. 75 is a graph of a ¹³C NMR measurement on 3,6-diiodo-9-octylcarbazole obtained in Example 22.

FIG. 76 is a graph of a ¹H NMR measurement on the 3,6-diiodo-9-octylcarbazole obtained in Example 22.

FIG. 77 is a graph of a ¹H NMR measurement on the 3,6-diiodo-9-octylcarbazole obtained in Example 22.

FIG. 78 is a graph of a ¹³C NMR measurement on a carbazole-silane compound obtained in Example 22.

FIG. 79 is a graph of a ¹H NMR measurement on the carbazole-silane compound obtained in Example 22.

FIG. 80 is a graph of a ¹H NMR measurement on the carbazole-silane compound obtained in Example 22.

FIG. 81 is a graph showing an X-ray diffraction pattern of a Carb-HMM-Acid-film obtained in Example 23.

FIG. 82 is a graph showing a fluorescence spectrum and an excitation spectrum of the Carb-HMM-Acid-film obtained in Example 23.

FIG. 83 is a graph showing a fluorescence spectrum and an excitation spectrum of a Carb-Acid-film obtained in Example 24.

FIG. 84 is a graph showing an X-ray diffraction pattern of a Carb-HMM-Acid obtained in Example 25.

FIG. 85 is a graph showing a fluorescence spectrum and an excitation spectrum of the Carb-HMM-Acid obtained in Example 25.

FIG. 86 is a graph showing an X-ray diffraction pattern of a Carb-HMM-Base obtained in Example 26.

FIG. 87 is a graph showing a fluorescence spectrum and an excitation spectrum of the Carb-HMM-Base obtained in Example 26.

FIG. 88 is a graph showing a fluorescence spectrum and an excitation spectrum of an Mcarb-Acid-film obtained in Example 27.

FIG. 89 is a graph of a ¹H NMR measurement on a quinacridone-silane compound obtained in Example 28.

FIG. 90 is a graph showing a UV spectrum of the quinacridone-silane compound obtained in Example 28.

FIG. 91 is a graph showing a UV spectrum of the quinacridone-silane compound obtained in Example 28.

FIG. 92 is a graph showing a fluorescence spectrum of the quinacridone-silane compound obtained in Example 28.

FIG. 93 is a graph showing an excitation spectrum of the quinacridone-silane compound obtained in Example 28.

FIG. 94 is a graph of a ¹H NMR measurement on a carbazole-silane compound obtained in Example 33.

FIG. 95 is a graph of a ¹H NMR measurement on the carbazole-silane compound obtained in Example 33.

FIG. 96 is a graph of a ¹H NMR measurement on a carbazole-silane compound obtained in Example 34.

FIG. 97 is a graph of a ¹³C NMR measurement on the carbazole-silane compound obtained in Example 34.

FIG. 98 is a graph of a ¹H NMR measurement on a fluorene-silane compound obtained in Example 35.

FIG. 99 is a graph of a ¹³C NMR measurement on the fluorene-silane compound obtained in Example 35.

### DETAILED DESCRIPTION OF THE PREFERRED EMBODIMENTS

Hereinafter, the present invention will be specifically described in line with preferred embodiments thereof.

### [Bridged Organosilane (i) and Production Method Thereof]

A preferred bridged organosilane (i) as the bridged organosilane of the present invention is a fluorene-silane compound expressed by the above-described general formula (29).

In the fluorene-silane compound, X²— in the general formula (29) is a substituent selected from the group consisting of substituents expressed by the general formulae (2) to (4). From the viewpoint of easiness in the polymerization of a monomer to be used in a sol-gel reaction, X²— is preferably a substituent in which R¹ in the general formulae (2) to (4) is a methyl or ethyl group and a substituent in which n is 3. Meanwhile, from the viewpoint of purification of the compound, n in the general formulae (2) to (4) is preferably 0 or 1. Moreover, from the viewpoints of easiness of synthesizing a mesoporous material and thermal stability of the compound, X²— is preferably a substituent expressed by the following formula:

—Si(OR¹)₃.

Y³< in the general formula (29) is a substituent selected from the group consisting of substituents expressed by the general formulae (7) to (11) and (30). From the viewpoints of chemical stability of the compound and easiness in the synthesis, R³ and R⁴ in the general formula (8) are preferably any one of alkyl groups having 1 to 22 (more preferably, 1 to 18) carbon atoms, a phenyl group, and a hydroxy group, and more preferably any one of a dodecyl group, a methyl group, an ethyl group, and a propyl group. Moreover, from the viewpoint of easiness in the synthesis, R⁵ in the general formula (11) is preferably any one of alkyl groups having 1 to 22 (more preferably, 1 to 18) carbon atoms, perfluoroalkyl groups having 1 to 22 (more preferably, 1 to 18) carbon atoms and aryl groups having 6 to 8 carbon atoms, and more preferably any one of a dodecyl group, a methyl group, an ethyl group, a perfluorodecyl group, a perfluoroisononyl group, and a phenyl group. Furthermore, from the viewpoint of easiness in the derivatization, Y³< is preferably a substituent expressed by the following formula:

H₂C<.

Next, a description will be given of a preferred method which allows the production of the bridged organosilane (i) as the bridged organosilane of the present invention (hereinafter, referred to as a “bridged organosilane (i) production method”). As described above, in the bridged organosilane (i) production method, which is the preferred production method of the bridged organosilane of the present invention, a fluorene compound expressed by the general formula (55) is caused to react with a silane compound expressed by the general formula (54) to obtain the bridged organosilane (i).

A fluorene compound used in the bridged organosilane (i) production method, which is the preferred production method of the bridged organosilane of the present invention is dihalogenated fluorene, dihydroxylated fluorene, or difluoromethylsulfonated fluorene, as expressed by the general formula (55). A halogen atom in the dihalogenated fluorene is preferably a bromine atom or an iodine atom from the viewpoint of easiness to cause a cross-coupling reaction. Moreover, a fluoromethylsulfonate group in the difluoromethylsulfonated fluorene is preferably a trifluoromethylsulfonate group from the viewpoint of easiness to cause an oxidative addition. Furthermore, of these fluorene compounds, 2,7-dibromofluorene can be used more preferably from the viewpoint of easiness in the synthesis.

Meanwhile, a silane compound used in the bridged organosilane (i) production method, which is the preferred production method of the bridged organosilane of the present invention is a silane compound expressed by the general formula (54). In the silane compound, R¹ is preferably a methyl group or an ethyl group from the viewpoint of easiness in handling of the compound.

Hereinbelow, a description will be given of a preferred embodiment of the bridged organosilane (i) production method. Specifically, firstly, the fluorene compound is mixed with a [Rh(CH₃CN)₂(cod)]BF₄ complex and Bu₄NI under a nitrogen atmosphere and a temperature condition of room temperature, and then added with a solvent to obtain a mixed liquid. Subsequently, the mixed liquid is added with triethylamine and dimethylformamido (DMF), thus a mixed solution is obtained. Thereafter, HSi(OEt)₃ is added dropwise thereto under a temperature condition of 0° C., and thoroughly stirred for 2 hours under a temperature condition of 80° C. Thereby, a crude product is obtained. After that, the solvent is removed, and the resultant crude product is purified, and thus a bridged organosilane can be obtained.

The solvent mixed with the fluorene compound includes dimethylformamide (DMF), acetonitrile, N-methyl-2-pyrrolidone (NMP) and dioxane. Meanwhile, the method of purifying the crude product is not particularly limited, and the example includes a synthesis method in which the crude product is dissolved in ether and then filtered through activated carbon.

Hereinabove, the description has been given of the preferred embodiment of the bridged organosilane (i) production method. In the present invention, the preferred bridged organosilane (i) production method is not limited to this. For example, the bridged organosilane obtained according to the above-described preferred embodiment of the bridged organosilane (i) production method is a bridged organosilane in which only alkoxide is bound to the silane. However, in the case to produce a bridged organosilane having the silane bound to an allyl group, it is possible to adopt the following method, as alternative production method. Specifically, in the method, after the crude product is obtained as in the above-described method adopted in the preferred embodiment of the bridged organosilane (i) production method, the crude product is further allylated and then purified to obtain a bridged organosilane.

The allylation method is not particularly limited, and the following method, for example, can be preferably adopted. Specifically, firstly, after the crude product is obtained as in the above-described method adopted in the preferred embodiment of the bridged organosilane (i) production method, an allylating agent, such as allylmagnesium bromide [CH₂═CH—CH₂MgBr], is added to the crude product under a nitrogen atmosphere and a temperature condition of approximately −10° C. to 0° C. to obtain a mixture. Then, the obtained mixture is thoroughly stirred under a room temperature condition (approximately 25° C.) for approximately 5 hours to 8 hours. Subsequently, the mixture is added with water under a temperature condition of approximately −10° C. to 0° C. to terminate the reaction. Thereafter, the pH of the mixture is adjusted to 7 by adding a solution, such as hydrochloric acid. After that, the resultant mixture is washed with a washing solution (for example, NaHCO₃ and NaCl) and then dried. Thereby, the crude product is allylated, and an allylated reaction product can be obtained. Subsequently, the allylated reaction product is purified, and thus it is possible to produce a bridged organosilane with the silane bound to an allyl group.

### [Bridged Organosilane (ii) and Production Method Thereof]

A preferred bridged organosilane (ii) as the bridged organosilane of the present invention is a pyrene-silane compound expressed by the general formula (31) or (32).

In the pyrene-silane compound, X³— in the general formula (31) or (32) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint in the purification of the compound, n in the general formula (2) is preferably 0 or 1.

Next, a description will be given of a preferred method which allows the production of the bridged organosilane (ii) as the bridged organosilane of the present invention (hereinafter, referred to as a “bridged organosilane (ii) production method”). As described above, in the bridged organosilane (ii) production method, which is the preferred production method of the bridged organosilane of the present invention, a pyrene compound expressed by the general formula (57) or (58) is caused to react with a silane compound expressed by the general formula (54) to obtain the bridged organosilane (ii). For the bridged organosilane (ii) production method, it is possible to adopt the same method as the above-described bridged organosilane (i) production method except that the pyrene compound expressed by the general formula (57) or (58) is used in place of the fluorene compound expressed by the general formula (55).

The pyrene compound used in the bridged organosilane (ii) production method, which is the preferred production method of the bridged organosilane of the present invention, is dihalogenated pyrene, dihydroxylated pyrene, or difluoromethylsulfonated pyrene, as expressed by the general formula (57) or (58). A halogen atom in the dihalogenated pyrene is preferably a bromine atom or an iodine atom from the viewpoint of easiness to cause a cross-coupling reaction. Moreover, a fluoromethylsulfonate group in the difluoromethylsulfonated pyrene is preferably a trifluoromethylsulfonate group from the viewpoint of easiness to cause an oxidative addition. Furthermore, of these pyrene compounds, a dibromo compound can be used more preferably from the viewpoint of easiness in the synthesis.

[Bridged Organosilane (iii) and Production Method Thereof]

A preferred bridged organosilane (iii) as the bridged organosilane of the present invention is an acridine-silane compound expressed by the general formula (33), (34) or (35).

In the acridine-silane compound, X³— in the general formula (33), (34) or (35) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint of purifying the compound, n in the general formula (2) is preferably 0 or 1. Moreover, from the viewpoints of easiness in the synthesis, R⁶— in the general formula (34) is preferably any one of alkyl groups having 1 to 22 (more preferably, 1 to 18) carbon atoms, perfluoroalkyl groups having 1 to 22 (more preferably, 1 to 18) carbon atoms, and aryl groups having 6 to 8 carbon atoms, and more preferably any one of a dodecyl group, a methyl group, an ethyl group, a perfluorodecyl group, a perfluoroisononyl group, and a phenyl group. Furthermore, from the viewpoints of chemical stability of the compound and easiness in the synthesis thereof, R⁷ and R⁸ in the general formula (35) are preferably alkyl groups having 1 to 22 (more preferably, 1 to 18) carbon atoms, perfluoroalkyl groups having 1 to 22 (more preferably, 1 to 18) carbon atoms, a phenyl group and a hydroxy group, and more preferably any one of a dodecyl group, a methyl group, an ethyl group, a propyl group, a perfluorodecyl group, and a perfluoroisononyl group.

Next, a description will be given of a preferred method which allows the production of the bridged organosilane (iii) as the bridged organosilane of the present invention (hereinafter, referred to as a “bridged organosilane (iii) production method”). As described above, in the bridged organosilane (iii) production method, which is the preferred production method of the bridged organosilane of the present invention, an acridine compound expressed by the general formula (59), (60) or (61) is caused to react with a silane compound expressed by the general formula (54) to obtain the bridged organosilane (iii). For the bridged organosilane (iii) production method, it is possible to adopt the same method as the above-described bridged organosilane (i) production method except that the acridine compound expressed by the general formula (59), (60) or (61) is used in place of the fluorene compound expressed by the general formula (55).

The acridine compound used in the bridged organosilane (iii) production method, which is the preferred production method of the bridged organosilane of the present invention, is dihalogenated acridine, dihydroxylated acridine, or difluoromethylsulfonated acridine, as expressed by the general formula (59), (60) or (61). A halogen atom in the dihalogenated acridine is preferably a bromine atom or an iodine atom from the viewpoint of easiness to cause a cross-coupling reaction. Moreover, a fluoromethylsulfonate group in the difluoromethylsulfonated acridine is preferably a trifluoromethylsulfonate group from the viewpoint of easiness to cause an oxidative addition. Furthermore, of these acridine compounds, a dibromo compound can be used more preferably from the viewpoint of easiness in the synthesis.

In the bridged organosilane (iii) production method, which is the preferred production method of the bridged organosilane of the present invention, it is possible to include a step of causing an acridine compound raw material expressed by the following general formula (72) or (73):

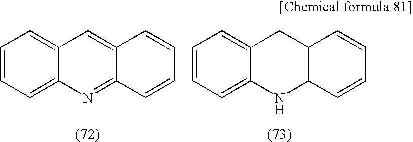

to react with benzyltriethylammonium tribromide [BTEABr₃] expressed by the following general formula (74):

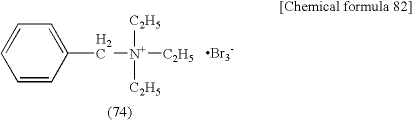

thereby to obtain an acridine compound expressed by the following general formula (75), (76) or (77):

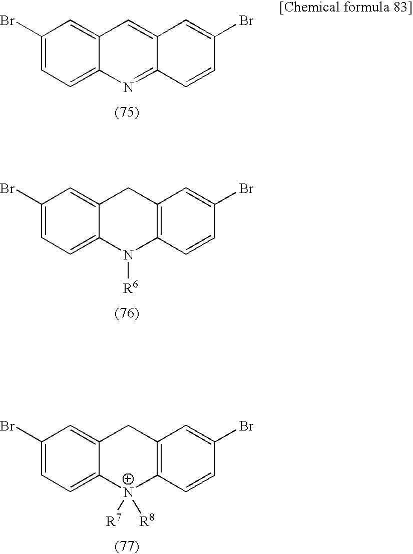

In other words, in the bridged organosilane (iii) production method, which is the preferred production method of the bridged organosilane of the present invention, the bridged organosilane can be produced by using the acridine compound obtained from the acridine compound raw material which has been subjected to dibromination with the BTEABr₃.

The dibromination method is not particularly limited, and the example includes the following method. Specifically, the acridine compound raw material and the BTEABr₃ are prepared, and added with an organic solvent, such as methanol and ethanol. The resultant mixture is refluxed under a temperature condition of approximately 75° C. to 85° C. for approximately 2 hours. Then, the mixture is cooled to room temperature (approximately 25° C.). In this method, a dibrominated acridine compound can be obtained through filtration subsequent to the dibromination.

It should be noted that the BTEABr₃ production method is not particularly limited, and the following method can be preferably adopted as an example. Firstly, in an open system, the mixture of benzyltriethylammonium chloride and sodium bromide is added with ion-exchanged water, and the solution thus obtained is stirred to dissolve the mixture. Then, dichloromethane is added to the solution, and the resultant mixture is vigorously stirred to mix the organic phase and aqueous phase. Subsequently, the mixture is cooled to approximately 0° C., and added dropwise with hydrogen bromide using a dropping funnel. After the resultant is stirred, the organic phase and the aqueous phase are separated, and the aqueous phase is extracted several times with dichloromethane. Thereafter, the organic phase thus obtained is dried, and the residual solid is recrystallized by using a solvent of dichloromethane and diethyl ether with a volumetric ratio of 5:1. Thus, BTEABr₃ can be obtained.

### [Bridged Organosilane (IV) and Production Method Thereof]

A preferred bridged organosilane (iv) as the bridged organosilane of the present invention is an acridone-silane compound expressed by the general formula (36).

In the acridone-silane compound, X³— in the general formula (36) is a substituent selected from the substituent group expressed by the general formula (2). From the viewpoint of easiness in the polymerization of a monomer to be used in a sol-gel reaction, X³— is preferably a substituent where R¹ in the general formula (2) is a methyl or ethyl group, and X³— is preferably a substituent where n is 3. Meanwhile, from the viewpoint of the purification of the compound, n in the general formula (2) is preferably 0 or 1.

Next, a description will be given of a preferred method which allows the production of the bridged organosilane (iv) as the bridged organosilane of the present invention (hereinafter, referred to as a “bridged organosilane (iv) production method”). As described above, in the bridged organosilane (iv) production method, which is the preferred production method of the bridged organosilane of the present invention, an acridone compound expressed by the general formula (62) is caused to react with a silane compound expressed by the general formula (54) to obtain the bridged organosilane (iv). For the bridged organosilane (iv) production method, it is possible to adopt the same method as the above-described bridged organosilane (i) production method except that the acridone compound expressed by the general formula (62) is used in place of the fluorene compound expressed by the general formula (55).

The acridone compound used in the bridged organosilane (iv) production method, which is the preferred production method of the bridged organosilane of the present invention, is dihalogenated acridone, dihydroxylated acridone, or difluoromethylsulfonated acridone, as expressed by the general formula (62). A halogen atom in the dihalogenated acridone is preferably a bromine atom or an iodine atom from the viewpoint of easiness to cause a cross-coupling reaction. Moreover, a fluoromethylsulfonate group in the difluoromethylsulfonated acridone is preferably a trifluoromethylsulfonate group from the viewpoint of easiness to cause an oxidative addition. Furthermore, of these acridone compounds, a dibromo compound can be used more preferably from the viewpoint of easiness in the synthesis.

In the bridged organosilane (iv) production method, which is the preferred production method of the bridged organosilane of the present invention, it is possible to include a step of causing an acridone compound raw material expressed by the following general formula (78):

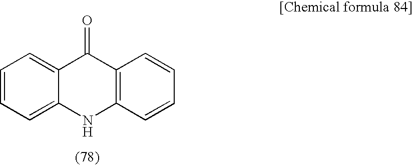

to react with benzyltriethylammonium tribromide expressed by the following general formula (74):

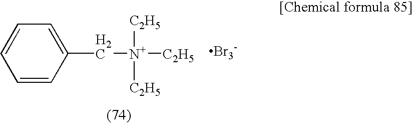

thereby to obtain an acridone compound expressed by the following general formula (79):

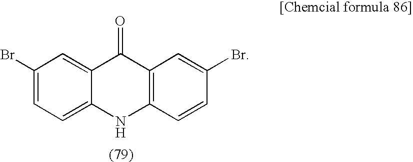

For the BTEABr₃ production method and the method (dibromination method) of causing BTEABr₃ to react with the acridone compound raw material, it is possible to adopt the same methods as described in the bridged organosilane (iii) production method

### [Bridged Organosilane (v) and Production Method Thereof]

A preferred bridged organosilane (v) as the bridged organosilane of the present invention is a quaterphenyl-silane compound expressed by the general formula (37).

In the quaterphenyl-silane compound, X³— in the general formula (37) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint in the purification of the compound, n in the general formula (2) is preferably 0 or 1.

Next, a description will be given of a preferred method which allows the production of the bridged organosilane (v) as the bridged organosilane of the present invention (hereinafter, referred to as a “bridged organosilane (v) production method”). As described above, in the bridged organosilane (v) production method, which is the preferred production method of the bridged organosilane of the present invention, a quaterphenyl compound expressed by the general formula (64) is caused to react with a silane compound expressed by the general formula (54) to obtain the bridged organosilane (v). For the bridged organosilane (v) production method, it is possible to adopt the same method as the above-described bridged organosilane (i) production method except that the quaterphenyl compound expressed by the general formula (64) is used in place of the fluorene compound expressed by the general formula (55).

The quaterphenyl compound used in the bridged organosilane (v) production method, which is the preferred production method of the bridged organosilane of the present invention, is dihalogenated quaterphenyl, dihydroxylated quaterphenyl, or difluoromethylsulfonated quaterphenyl, as expressed by the general formula (64). A halogen atom in the dihalogenated quaterphenyl is preferably a bromine atom or an iodine atom from the viewpoint of easiness to cause a cross-coupling reaction. Moreover, a fluoromethylsulfonate group in the difluoromethylsulfonated quaterphenyl is preferably a trifluoromethylsulfonate group from the viewpoint of easiness to cause an oxidative addition. Furthermore, of these quaterphenyl compounds, a dibromo compound can be used more preferably from the viewpoint of easiness in the synthesis.

### [Bridged Organosilane (vi) and Production Method Thereof]

A preferred bridged organosilane (vi) as the bridged organosilane of the present invention is an anthracene-silane compound expressed by the general formula (38) or (39), which is a compound with silanes bound to carbons at the 2- and 6-positions of the anthracene.

In the anthracene-silane compound, X³— in the general formula (38) or (39) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint of purifying the compound, n in the general formula (2) is preferably 0 or 1.

Y²< in the general formula (38) or (39) is a substituent expressed by the general formula (10) or (11). From the viewpoint of easiness in the synthesis, R⁵ in the general formula (11) is preferably any one of alkyl groups having 1 to 22 (more preferably, 1 to 18) carbon atoms, perfluoroalkyl groups having 1 to 22 (more preferably, 1 to 18) carbon atoms, and aryl groups having 6 to 8 carbon atoms, and more preferably any one of a dodecyl group, a methyl group, an ethyl group, a perfluorodecyl group, a perfluoroisononyl group, and a phenyl group.

Next, a description will be given of a preferred method which allows the production of the bridged organosilane (vi) as the bridged organosilane of the present invention (hereinafter, referred to as a “bridged organosilane (vi) production method”). As described above, in the bridged organosilane (vi) production method, which is the preferred production method of the bridged organosilane of the present invention, an anthracene compound expressed by the general formula (64) is caused to react with a silane compound expressed by the general formula (54) to obtain the bridged organosilane (vi). For the bridged organosilane (vi) production method, it is possible to adopt the same method as the above-described bridged organosilane (i) production method except that the anthracene compound expressed by the general formula (64) is used in place of the fluorene compound expressed by the general formula (55).

The anthracene compound used in the bridged organosilane (vi) production method, which is the preferred production method of the bridged organosilane of the present invention, is dihalogenated anthracene, dihydroxylated anthracene, or difluoromethylsulfonated anthracene, as expressed by the general formula (64). A halogen atom in the dihalogenated anthracene is preferably a bromine atom or an iodine atom from the viewpoint of easiness in the synthesis. Moreover, a fluoromethylsulfonate group in the difluoromethylsulfonated anthracene is preferably a trifluoromethylsulfonate group from the viewpoint of easiness to cause an oxidative addition. Furthermore, of these anthracene compounds, a dibromo compound can be used more preferably from the viewpoint of easiness in the synthesis.

In the bridged organosilane (vi) production method, which is the preferred production method of the bridged organosilane of the present invention, it is possible to include a step (i) for reducing an anthraquinone compound raw material expressed by the following general formula (80):

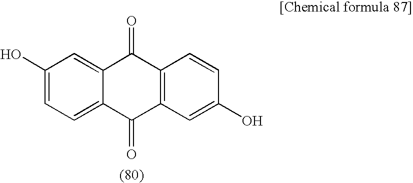

to obtain an anthracene compound precursor expressed by the following general formula (81):

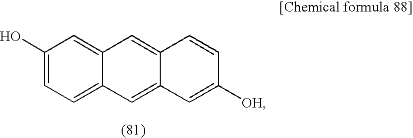

and a step (ii) for causing the anthracene compound precursor to react with trifluoromethanesulfonic anhydride thereby to obtain an anthracene compound expressed by the following general formula (82):

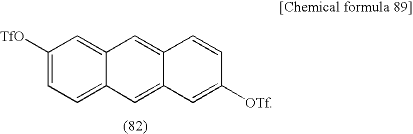

The method of reducing an anthraquinone compound raw material in the step (i) is not particularly limited, and it is possible to adopt a known method as appropriate. A preferred method of reducing an anthraquinone compound raw material can include the following method. Specifically, firstly, aluminum is put into a reaction container, and a mercury chloride aqueous solution is added thereto. The mixture is stirred approximately 1 minute to 2 minutes. Then, distilled water, ethanol and concentrated ammonia water are sequentially added into the reaction container. Subsequently, the anthraquinone compound raw material is added thereto under a nitrogen atmosphere (nitrogen flow), and the resultant is stirred under a temperature condition of 60° C. to 65° C. Thereby, the anthraquinone compound raw material can be reduced.

In the step (ii), the method of causing the anthracene compound precursor to react with trifluoromethanesulfonic anhydride is not particularly limited, and the following method can be preferably adopted as an example. Specifically, in the preferred method of causing the anthracene compound precursor to react with trifluoromethanesulfonic anhydride, firstly, the anthracene compound precursor obtained in the step (i) is dissolved in dichloromethane to prepare a solution. The solution is added with pyridine, and then added dropwise with trifluoromethanesulfonic anhydride under a temperature condition of −10° C. to 0° C. The resultant mixture is vigorously stirred for approximately 15 hours to 20 hours. Subsequently, the aqueous phase is extracted with dichloromethane, and thereafter the organic phase is washed with a saturated NaHCO₃ aqueous solution and brine, and then dried. In this method, the reaction between the anthracene compound precursor and trifluoromethanesulfonic anhydride is successfully achieved to obtain the anthracene compound expressed by the general formula (82).

[Bridged Organosilane (vii) and Production Method Thereof]

A preferred bridged organosilane (vii) as the bridged organosilane of the present invention is a carbazole-silane compound expressed by the general formula (40) or (41).

In the carbazole-silane compound, X¹— in the general formula (40) or (41) is a substituent selected from the group consisting of substituents expressed by the general formulae (2) to (5). From the viewpoint of easiness in the polymerization of a monomer to be used in a sol-gel reaction, X⁴— is preferably a substituent in which R¹ in the general formulae (2) to (5) is a methyl or ethyl group, a substituent in which n is 3, and a substituent in which m is 0. Meanwhile, from the viewpoint in the purification of the compound, in the general formulae (2) to (5), n is preferably 0 or 1, and m is preferably 0. Note that, the reason why the preferable value of m is 0 as mentioned above is that an acrylic acid derivative serving as the raw material is readily available as a commercial product. Moreover, from the viewpoints of easiness in the synthesis, R⁹ in the general formula (40) is preferably any one of alkyl groups having 1 to 22 (more preferably, 1 to 18) carbon atoms, perfluoroalkyl groups having 1 to 22 (more preferably, 1 to 18) carbon atoms, and aryl groups having 6 to 8 carbon atoms, and more preferably any one of a dodecyl group, a methyl group, an ethyl group, a perfluorodecyl group, a perfluoroisononyl group, and a phenyl group. Furthermore, from the viewpoints of the chemical stability of the compound and of easiness of the synthesis, R¹⁰ and R¹¹ in the general formula (41) are preferably alkyl groups having 1 to 22 (more preferably, 1 to 18) carbon atoms, perfluoroalkyl groups having 1 to 22 (more preferably, 1 to 18) carbon atoms, and a phenyl group, and more preferably a dodecyl group, a methyl group, an ethyl group, a propyl group, a perfluorodecyl group, and a perfluoroisononyl group.

Next, a description will be given of a preferred method which allows the production of the bridged organosilane (vii) as the bridged organosilane of the present invention (hereinafter, referred to as a “bridged organosilane (vii) production method”). As described above, in the bridged organosilane (vii) production method, which is the preferred production method of the bridged organosilane of the present invention, a carbazole compound expressed by the general formula (65) or (66) is caused to react with a silane compound expressed by the general formula (54) to obtain a bridged organosilane (vii). For the bridged organosilane (vii) production method, it is possible to adopt the same method as the above-described bridged organosilane (i) production method except that the carbazole compound expressed by the general formula (65) or (66) is used in place of the fluorene compound expressed by the general formula (55).

Additionally, the carbazole compound used in the bridged organosilane (vii) production method, which is the preferred production method of the bridged organosilane of the present invention, is dihalogenated carbazole, dihydroxylated carbazole, or difluoromethylsulfonated carbazole, as expressed by the general formula (65) or (66). A halogen atom in the dihalogenated carbazole is preferably a bromine atom or an iodine atom from the viewpoint of easiness to cause a cross-coupling reaction. Moreover, a fluoromethylsulfonate group in the difluoromethylsulfonated carbazole is preferably a trifluoromethylsulfonate group from the viewpoint of easiness to cause an oxidative addition. Furthermore, of these carbazole compounds, a dibromo compound and a diiodo compound can be used more preferably from the viewpoint of easiness in the synthesis.

In the bridged organosilane (vii) production method, which is the preferred production method of the bridged organosilane of the present invention, it is possible to include a step of causing a carbazole compound raw material expressed by the following general formula (83):

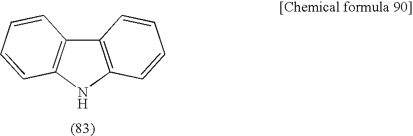

to react with bis(pyridine)iodonium tetrafluoroborate (IPy₂BF₄) thereby to obtain a carbazole compound expressed by the following general formula (84) or (85):

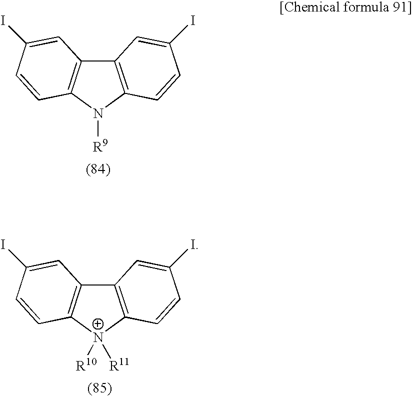

In other words, in the bridged organosilane (vii) production method, a bridged organosilane can be produced by using the carbazole compound obtained from the carbazole compound raw material which has been subjected to diiodization with the bis(pyridine)iodonium tetrafluoroborate.

The diiodization method is not particularly limited, and the example includes the following method. Specifically, the carbazole compound raw material and bis(pyridine)iodonium tetrafluoroborate are prepared, and the mixture thereof is added with dichloromethane under a nitrogen atmosphere. Trifluoromethanesulfonic acid is further added dropwise to the mixture under a temperature condition of approximately 0° C. Then, the resultant mixture is stirred at room temperature for an extended period of time (preferably, approximately 10 hours to 40 hours).

[Bridged Organosilane (viii) and Production Method Thereof]

A preferred bridged organosilane (viii) as the bridged organosilane of the present invention is a quinacridone-silane compound expressed by the general formula (42).

In the quinacridone-silane compound, X³— in the general formula (42) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint in the purification of the compound, n in the general formula (2) is preferably 0 or 1.

Moreover, from the viewpoint of easiness in the synthesis, R¹² and R¹³ in the general formula (42) are preferably alkyl groups having 1 to 22 (more preferably, 1 to 18) carbon atoms, perfluoroalkyl groups having 1 to 22 (more preferably, 1 to 18) carbon atoms, and aryl groups having 6 to 8 carbon atoms, and more preferably a dodecyl group, a methyl group, an ethyl group, a perfluorodecyl group, a perfluoroisononyl group, and a phenyl group.

Next, a description will be given of a preferred method which allows the production of the bridged organosilane (viii) as the bridged organosilane of the present invention (hereinafter, referred to as a “bridged organosilane (viii) production method”). As described above, in the bridged organosilane (viii) production method, which is the preferred production method of the bridged organosilane of the present invention, a quinacridone compound expressed by the general formula (67) is caused to react with a silane compound expressed by the general formula (54) to obtain the bridged organosilane (viii). For the bridged organosilane (viii) production method, it is possible to adopt the same method as the above-described bridged organosilane (i) production method except that the quinacridone compound expressed by the general formula (67) is used in place of the fluorene compound expressed by the general formula (55).

The quinacridone compound used in the bridged organosilane (viii) production method, which is the preferred production method of the bridged organosilane of the present invention, is dihalogenated quinacridone, dihydroxylated quinacridone, or difluoromethylsulfonated quinacridone, as expressed by the general formula (67). A halogen atom in the dihalogenated quinacridone is preferably a bromine atom or an iodine atom from the viewpoint of the synthesis. Moreover, a fluoromethylsulfonate group in the difluoromethylsulfonated quinacridone is preferably a trifluoromethylsulfonate group from the viewpoint of easiness to cause an oxidative addition. Furthermore, of these quinacridone compounds, a dibromo compound can be used more preferably from the viewpoint of easiness in the synthesis.

### [Bridged Organosilane (ix) and Production Method Thereof]

A preferred bridged organosilane (ix) as the bridged organosilane of the present invention is a rubrene-silane compound expressed by the general formula (43).

In the rubrene-silane compound, X³— in the general formula (43) or (44) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint in the purification of the compound, n in the general formula (2) is preferably 0 or 1.

Next, a description will be given of a preferred method which allows the production of the bridged organosilane (ix) as the bridged organosilane of the present invention (hereinafter, referred to as a “bridged organosilane (ix) production method”). As described above, in the bridged organosilane (ix) production method, which is the preferred production method of the bridged organosilane of the present invention, a rubrene compound expressed by the general formula (68) or (69) is caused to react with a silane compound expressed by the general formula (54) to obtain the bridged organosilane (ix). For the bridged organosilane (ix) production method, it is possible to adopt the same method as the above-described bridged organosilane (i) production method except that the rubrene compound expressed by the general formula (68) or (69) is used in place of the fluorene compound expressed by the general formula (55).

The rubrene compound used in the bridged organosilane (ix) production method, which is the preferred production method of the bridged organosilane of the present invention, is dihalogenated or tetrahalogenated rubrene, dihydroxylated or tetrahydroxylated rubrene, or difluoromethylsulfonated or tetrafluoromethylsulfonated rubrene, as expressed by the general formula (68) or (69). A halogen atom in the dihalogenated or tetrahalogenated rubrene is preferably a bromine atom or an iodine atom from the viewpoint of the synthesis. Moreover, a fluoromethylsulfonate group in the difluoromethylsulfonated or tetrafluoromethylsulfonated rubrene is preferably a trifluoromethylsulfonate group from the viewpoint of easiness to cause an oxidative addition. Furthermore, of these rubrene compounds, those with a dibromo or tetrabromo compound and a diiode or tetraiodo compound can be used more preferably from the viewpoint of easiness in the synthesis.

### [Bridged Organosilane (x) and Production Method Thereof]

A preferred bridged organosilane (x) as the bridged organosilane of the present invention is a 1,4-alkyloxy-2,5-phenylethenylbenzene-silane compound expressed by the general formula (45).

In the 1,4-alkyloxy-2,5-phenylethenylbenzene-silane compound, X³— in the general formula (45) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint in the purification of the compound, n in the general formula (2) is preferably 0 or 1.

Additionally, from the viewpoint of easiness in the synthesis, R¹⁴ and R¹⁵ in the general formula (45) are preferably alkyl groups having 1 to 22 (more preferably, 1 to 18) carbon atoms, perfluoroalkyl groups having 1 to 22 (more preferably, 1 to 18) carbon atoms, and aryl groups having 6 to 8 carbon atoms and more preferably any one of a dodecyl group, a methyl group, an ethyl group, a hexyl group, a perfluorodecyl group, a perfluoroisononyl group, and a phenyl group.

Next, a description will be given of a preferred method which allows the production of the bridged organosilane (x) as the bridged organosilane of the present invention (hereinafter, referred to as a “bridged organosilane (x) production method”). As described above, in the bridged organosilane (x) production method, which is the preferred production method of the bridged organosilane of the present invention, a 1,4-alkyloxy-2,5-phenylethenylbenzene compound expressed by the general formula (70) is caused to react with a silane compound expressed by the general formula (54) to obtain the bridged organosilane (x). For the bridged organosilane (x) production method, it is possible to adopt the same method as the above-described bridged organosilane (i) production method except that the 1,4-alkyloxy-2,5-phenylethenylbenzene compound expressed by the general formula (70) is used in place of the fluorene compound expressed by the general formula (55).

The 1,4-alkyloxy-2,5-phenylethenylbenzene compound used in the bridged organosilane (x) production method, which is the preferred production method of the bridged organosilane of the present invention, is dihalogenated 1,4-alkyloxy-2,5-phenylethenylbenzene, dihydroxylated 1,4-alkyloxy-2,5-phenylethenylbenzene, or difluoromethylsulfonated 1,4-alkyloxy-2,5-phenylethenylbenzene, as expressed by the general formula (70). A halogen atom in the dihalogenated 1,4-alkyloxy-2,5-phenylethenylbenzene is preferably a bromine atom or an iodine atom from the viewpoint of the synthesis. Moreover, a fluoromethylsulfonate group in the difluoromethylsulfonated 1,4-alkyloxy-2,5-phenylethenylbenzene is preferably a trifluoromethylsulfonate group from the viewpoint of easiness to cause an oxidative addition. Furthermore, of these 1,4-alkyloxy-2,5-phenylethenylbenzene compounds, a dibromo compound and an diiodo compound can be used more preferably from the viewpoint of easiness in the synthesis.

### [Bridged Organosilane (xi) and Production Method Thereof]

A preferred bridged organosilane (xi) as the bridged organosilane of the present invention is a triphenylamine-silane compound expressed by the general formula (46).

In the triphenylamine-silane compound, X³— in the general formula (46) is a substituent selected from the substituent group expressed by the general formula (2). From the viewpoint of easiness in the polymerization of a monomer to be used in a sol-gel reaction, X³— is preferably a substituent in which R¹ in the general formula (2) is a methyl or ethyl group and a substituent in which n is 3. Meanwhile, from the viewpoint of the purification of the compound, n in the general formula (2) is preferably 0 or 1.

Next, a description will be given of a preferred method which allows the production of the bridged organosilane (xi) as the bridged organosilane of the present invention (hereinafter, referred to as a “bridged organosilane (xi) production method”). As described above, in the bridged organosilane (xi) production method, which is the preferred production method of the bridged organosilane of the present invention, a triphenylamine compound expressed by the general formula (71) is caused to react with a silane compound expressed by the general formula (54) to obtain the bridged organosilane (xi). For the bridged organosilane (xi) production method, it is possible to adopt the same method as the above-described bridged organosilane (i) production method except that the triphenylamine compound expressed by the general formula (71) is used in place of the fluorene compound expressed by the general formula (55).

Additionally, the triphenylamine compound used in the bridged organosilane (xi) production method, which is the preferred production method of the bridged organosilane of the present invention, is trihalogenated triphenylamine, trihydroxylated triphenylamine, or trifluoromethylsulfonated triphenylamine, as expressed by the general formula (71). A halogen atom in the trihalogenated triphenylamine is preferably a bromine atom or an iodine atom from the viewpoint of the synthesis. Moreover, a fluoromethylsulfonate group in the difluoromethylsulfonated triphenylamine is preferably a trifluoromethylsulfonate group from the viewpoint of easiness to cause an oxidative addition. Furthermore, of these triphenylamine compounds, a tribromo compound and a triiodo body can be used more preferably from the viewpoint of easiness in the synthesis.

Moreover, in the bridged organosilane (xi) production method, which is the preferred production method of the bridged organosilane of the present invention, it is possible to include a step of causing triphenylamine to react with bis(pyridine)iodonium tetrafluoroborate (IPy₂BF₄) thereby to obtain a triphenylamine compound. In other words, in the bridged organosilane (xi) production method, bridged organosilane can be produced by using the triphenylamine compound obtained from the triphenylamine which has been subjected to triiodization with the bis(pyridine)iodonium tetrafluoroborate.

The triiodization method is not particularly limited, and the example includes the following method. Specifically, triphenylamine and bis(pyridine)iodonium tetrafluoroborate are prepared, and the mixture thereof is added with dichloromethane under a nitrogen atmosphere. Trifluoromethanesulfonic acid is further added dropwise to the mixture under a temperature condition of approximately 0° C. Then, the resultant mixture is stirred at room temperature for an extended period of time (preferably, approximately 10 hours to 40 hours).

Hereinabove, the description has been given of the preferred bridged organosilanes (i) to (xi) as the bridged organosilane of the present invention as well as the production methods thereof. These bridged organosilanes of the present invention can be used as a light-emitting material after being polymerized.

When being used as the light-emitting material, one of the bridged organosilanes of the present invention may be polymerized, or two or more thereof may be copolymerized. Moreover, when the bridged organosilane of the present invention is used as the light-emitting material, the bridged organosilane of the present invention may be copolymerized with an organosilicon compound composed of organic molecules emitting no fluorescence or phosphorescence. Hereinbelow, the bridged organosilane of the present invention and a monomer which is provided for copolymerization as necessary are collectively referred to as a “monomer”. Additionally, when the bridged organosilane of the present invention is copolymerized with the organosilicon compound composed of organic molecules exhibiting no fluorescence or phosphorescence and used as a light-emitting material, the percentage of the bridged organosilane of the present invention in the total monomer is preferably 1% or higher.

A polymer obtained by polymerizing the above-described monomer serves as an organosilica material having a backbone mainly composed of a silicon atom (Si), an oxygen atom (O), and a fluorescent molecule (X), such as fluorene, pyrene, acridine, acridone, quaterphenyl, anthracene, carbazole, quinacridone, and rubrene. Such an organosilica material has a highly-bridged mesh structure based on a backbone (—X—Si—O—) in which the silicon atom bound to the fluorescent molecule is bound to the oxygen atom.

The method of polymerizing the monomer is not particularly limited. It is preferable that the monomer be hydrolyzed and condensed under the presence of an acidic or basic catalyst upon using water or a mixture solvent of water and an organic solvent serving as a solvent. An organic solvent preferably used includes alcohol, acetone, and the like. When a mixture solvent is used, the content of the organic solvent is preferably in a range from approximately 5% by weight to 50% by weight. Moreover, an acidic catalyst to be used may be, for example, a mineral acid, such as hydrochloric acid, nitric acid, and sulfuric acid. When an acidic catalyst is used, the solution is preferably acidic at a pH of 6 or below (more preferably in a range from 2 to 5). Furthermore, a basic catalyst to be used may be, for example, sodium hydroxide, ammonium hydroxide, and potassium hydroxide. When a basic catalyst is used, the solution is preferably basic at a pH of 8 or higher (more preferably in a range from 9 to 11).

The content of the monomer in the polymerization step is preferably approximately 0.0055 mol/L to 0.33 mol/L in terms of silica concentration. The reaction conditions (temperature, duration, and the like) in the polymerization step are not particularly limited, and are selected appropriately in accordance with the monomer to be used, a targeted polymer, or the like. In general, it is preferable that the organosilicon compound be hydrolyzed and condensed at a temperature of approximately 0° C. to 100° C. for 1 hour to 48 hours.

Moreover, the polymer obtained by polymerizing the monomer (the polymer obtained by polymerizing the bridged organosilane of the present invention) generally has an amorphous structure. However, the polymer can have a periodic structure based on an ordered arrangement of the fluorescent molecules in accordance with the synthesis conditions. Although such periodicity depends on the molecular length of the monomer to be used, the periodicity of the periodic structure is preferably 5 nm or below. Such a periodic structure is maintained even after the monomer is polymerized. The formation of the periodic structure can be recognized by a peak appeared in a region where the d value is 5 nm or below in the X-ray diffraction (XRD) measurement. Note that, even when such a peak is not recognized in the XRD measurement, the periodic structure is partially formed in some cases. Such a periodic structure is generally formed with a layered structure to be described below, but not limited to this case.

In the case where the bridged organosilane of the present invention is used as the light-emitting material as described above, when the periodic structure based on the ordered arrangement of the fluorescent molecules is formed, the emission intensity tends to increase significantly. Furthermore, as a preferable synthesis condition for forming the periodic structure based on the ordered arrangement of the fluorescent molecules, for example, the solution preferably has a pH of 1 to 3 (acidic) or a pH of 10 to 12 (basic), and more preferably has a pH of 10 to 12 (basic). Such a periodic structure can be obtained in accordance with the method described in, for example, S. Inagaki et al., Nature, (2002), vol. 416, pp. 304 to 307.

Furthermore, pores can be formed in the obtained polymer (the polymer obtained by polymerizing the bridged organosilane of the present invention) by controlling the synthesis condition when the monomer is polymerized, or by mixing a surfactant to the bridged organosilane of the present invention. The solvent serves as a template to form a porous material having pores in the former case, while the micelle or liquid crystal structure of the surfactant serves as the template in the latter case.

Particularly, it is preferable to use a surfactant to be described below, since a mesoporous material having mesopores with a central pore diameter of 1 nm to 30 nm in a pore diameter distribution curve can be obtained. Note that the central pore diameter is a pore diameter at the maximum peak of the curve (pore diameter distribution curve). In this curve, values (dV/dD) obtained by differentiating a pore volume (V) by a pore diameter (D) are plotted to corresponding pore diameter (D). The central pore diameter can be obtained by the method described below. Specifically, the porous material is cooled to a liquid nitrogen temperature (−196° C.). Then, a nitrogen gas is introduced to the porous material, and an absorbed amount of the nitrogen gas is determined with a volumetrical method or a gravimetrical method. Subsequently, the pressure of the nitrogen gas being introduced is gradually increased. Thereafter, the amount of nitrogen gas adsorbed is plotted to each equilibrium pressure, thereby an adsorption isotherm is obtained. Based on this adsorption isotherm, a pore diameter distribution curve can be acquired by a calculation method, such as a Cranston-Inklay method, a Pollimore-Heal method, and a BJH method.

It is preferable that at least 60% of the total pore volume of the mesoporous material be included within a range of ±40% of the central pore diameter in the pore diameter distribution curve. Such a mesoporous material satisfying this condition has highly uniform diameters of the pores thereof. Meanwhile, the specific surface area of the mesoporous material is not particularly limited, and is preferably 400 m²/g or above. The specific surface area can be calculated as a BET specific surface area on the basis of the adsorption isotherm by a BET isothermal adsorption equation.

Furthermore, the mesoporous material preferably has one or more peaks at a diffraction angle corresponding to a d value in a range from 1.5 nm to 30.5 nm in the XRD pattern. An X-ray diffraction peak indicates that a periodic structure of a d value corresponding to the peak angle is present in the sample. Accordingly, the fact that one or more peaks are present at a diffraction angle corresponding to a d value in a range from 1.5 nm to 30.5 nm means that the pores are orderly arranged at intervals in a range from 1.5 nm to 30.5 nm.

The pores in the mesoporous material are formed not only on the surface of the porous material but also in the inside thereof. The pore arrangement state (pore arrangement structure, or simply structure) in the porous material is not particularly limited, and is preferably of a 2d-hexagonal structure, a 3d-hexagonal structure, or a cubic structure. The pore arrangement structure may be a disordered pore arrangement structure.

In this case, the phrase that the porous material has a hexagonal pore arrangement structure means that the arrangement of the pores is of a hexagonal structure (see: S. Inagaki et. al., J. Chem. Soc., Chem. Commun., p. 680 (1993); S. Inagaki et al., Bull. Chem. Soc. Jpn., 69, p. 1449 (1996); and Q. Huo et al., Science, 268, p. 1324 (1995)). Moreover, the phrase that the porous material has a cubic pore arrangement structure means that the arrangement of the pores is of a cubic structure (see: J. C. Vartuli et al., Chem. Mater., 6, p. 2317 (1994); and Q. Huo et al., Nature, 368, p. 317 (1994)). In addition, the phrase that the porous material has a disordered pore arrangement structure means that the arrangement of the pores is irregular (see: P. T. Tanev et al., Science, 267, p. 865 (1995); S. A. Bagshaw et al., Science, 269, p. 1242 (1995); and R. Ryoo et al., J. Phys. Chem., 100, p. 17718 (1996)). Furthermore, the cubic structure preferably has a Pm-3n, Ia-3d, Im-3m, or Fm-3m symmetry. The symmetrical property is to be determined on the basis of the notation of a space group.

In the case where the light-emitting material made of the bridged organosilane of the present invention has pores, it allow the porous material to adsorb (by physical adsorption and/or chemical bonding) a different light-emitting compound to be described below. In such a case, an energy is transferred from the above-described florescent molecule to the different light-emitting compound, and accordingly the resultant porous material emits light which has a wavelength different from that of the original fluorescent molecule. Thereby, it is possible to obtain multiple color light emission in accordance with the combination of the introduced fluorescent molecule and light-emitting compound. Moreover, in the case where the periodic structure is formed in the pore wall of the porous material, the energy is more efficiently transferred from the florescent molecule in the pore wall to the different light-emitting compound, and, as a result, it is possible to achieve light emission at a strong intensity having the different wavelength. Furthermore, the introduction of a charge-transfer material to be described below into the pores of the porous material allows the fluorescent molecule in the pore wall to emit light more efficiently. To obtain the mesoporous material, it is desirable that the monomer (the bridged organosilane of the present invention) be polycondensed upon being added with a surfactant. This is because the added surfactant serves as a template to form mesopores when the monomer is polycondensed.

The surfactant used in obtaining the mesoporous material is not particularly limited, and may be any one of cationic, anionic, and nonionic surfactants. To be more specific, the surfactant includes: a chloride, a bromide, an iodide, and a hydroxide of alkyltrimethylammonium, alkyltriethylammonium, dialkyldimethylammonium, benzyl ammonium, and the like; and a fatty acid salt, alkylsulfonate, alkylphosphate, polyethylene oxide-based nonionic surfactant, primary alkylamine, and the like. These surfactants are used alone or in combination of two or more kinds.

Among the above surfactants, the polyethylene oxide-based nonionic surfactant includes ones having a hydrocarbon group as a hydrophobic component and a polyethylene oxide as a hydrophilic component, for example. Such a surfactant preferably used is expressed by a general formula, for example, CₙH₂ₙ₊₁(OCH₂CH₂)ₘOH where n is in a range from 10 to 30 and m is in a range from 1 to 30. As the surfactant, esters of sorbitan and a fatty acid, such as oleic acid, lauric acid, stearic acid, and palmitic acid, or compounds formed by adding polyethylene oxide to these esters can also be used.

Furthermore, as the surfactant, a triblock copolymer of polyalkylene oxide can also be used. Such surfactants include one made of polyethylene oxide (EO) and polypropylene oxide (PO), and expressed by a general formula (EO)ₓ(PO)_{y}(EO)ₓ. Here, x and y represent the numbers of repetitions of EO and PO, respectively. It is preferable that x be in a range from 5 to 110 and y be in a range from 15 to 70, and more preferable that x be in a range from 13 to 106 and y be in a range from 29 to 70. Such triblock copolymers include (EO)₁₉(PO)₂₉(EO)₁₉, (EO)₁₃(PO)₇₀(EO)₁₃, (EO)₅(PO)₇₀(EO)₅, (EO)₁₃(PO)₃₀(EO)₁₃, (EO)₂₀(PO)₃₀(EO)₂₀, (EO)₂₆(PO)₃₉(EO)₂₆, (EO)₁₇(PO)₅₆(EO)₁₇, (EO)₁₇(PO)₅₈(EO)₁₇, (EO)₂₀(PO)₇₀(EO)₂₀, (EO)₈₀(PO)₃₀(EO)₈₀, (EO)₁₀₆(PO)₇₀(EO)₁₀₆, (EO)₁₀₀(PO)₃₉(EO)₁₀₀, (EO)₁₉(PO)₃₃(EO)₁₉ and (EO)₂₆(PO)₃₆(EO)₂₆. These triblock copolymers are available from BASF Group, Sigma-Aldrich Corp., and the like. The triblock copolymer having desired x and y values can also be obtained in a small-scale production level.

It is also possible to use a star diblock copolymer formed by binding two chains of a polyethylene oxide (EO) chain-polypropylene oxide (PO) chain to each of two nitrogen atoms of ethylenediamine. Such star diblock copolymers include one expressed by a general formula ((EO)ₓ(PO)_{y})₂NCH₂CH₂N((PO)_{y}(EO)ₓ)₂ where x and y are the numbers of repetitions of EO and PO, respectively. It is preferable that x be in a range from 5 to 110 and y be in a range from 15 to 70, and more preferable that x be in a range from 13 to 106 and y be in a range from 29 to 70.

Among the above surfactants, a salt (preferably a halide salt) of alkyltrimethylammonium [CₚH₂ₚ₊₁N(CH₃)₃] is preferably used because a mesoporous material having a high crystallinity can be obtained by using this surfactant. In this case, the alkyltrimethylammonium more preferably has an alkyl group having 8 to 22 carbon atoms. Such alkyltrimethylammoniums include, for example, octadecyltrimethylammonium chloride, hexadecyltrimethylammonium chloride, tetradecyltrimethylammonium chloride, dodecyltrimethylammonium bromide, decyltrimethylammonium bromide, octyltrimethylammonium bromide, and docosyltrimethylammonium chloride.

In order to obtain a mesoporous material from the polymer produced by polymerizing the bridged organosilane of the present invention, the monomer is subjected to the polymerization reaction in a solution containing the surfactant. The concentration of the surfactant in the solution is preferably in a range from 0.05 mol/L to 1 mol/L. When the concentration is less than the lower limit, the formation of the pores tends to be incomplete. On the other hand, when the concentration exceeds the upper limit, the amount of the surfactant which is unreacted and left in the solution is increased, and therefore the uniformity of the pores tends to be decreased.

Then, the surfactant contained in the mesoporous material thus obtained may be removed. The method of removing the surfactant includes the following methods, for example: (i) a method of removing the surfactant in which the mesoporous material is immersed in an organic solvent (for example, ethanol) having a high solubility to the surfactant; (ii) a method of removing the surfactant in which the mesoporous material is calcined at 250° C. to 1000° C.; and (iii) an ion-exchange method in which the mesoporous material is immersed in an acidic solution and heated to exchange the surfactant with hydrogen ions.

The mesoporous material can also be obtained in accordance with the method described in, for example, Japanese Unexamined Patent Application Publication No. 2001-114790.

Advantages of making the obtained light-emitting material made of the bridged organosilane of the present invention into a porous material are: (i) that it is possible to obtain multiple color light emission by introducing a different light-emitting compound into the pores thereby to efficiently transfer an excitation energy of the pore wall to the light-emitting compound; (ii) that the durability of the light-emitting compound introduced into the pores is improved; and furthermore (iii) that the light extraction efficiency can be improved by reducing the refractive index of the light emitting layer.

The structure of the light-emitting material, which is made of the bridged organosilane of the present invention, further containing a different light-emitting compound is not particularly limited. The different light-emitting compound may be in any state of adsorbing, binding, filling, and mixing, in a nonporous or porous light-emitting material. The state of adsorbing refers to a state where the light-emitting compound is attached to particles of the light-emitting material or the surface of the film in the case where the light-emitting material is nonporous, and where the light-emitting compound is attached to the inner or outer surface of pores of a light-emitting material in the case where the light-emitting material is porous. The state of binding refers to a case where such an attachment involves a chemical bonding. The state of filling refers to a state where a different light-emitting compound exists in pores of a porous light-emitting material, and the different light-emitting compound need not be attached on the surface of the pores in this case. While a substance other than a different light-emitting compound is filled in pores, a different light-emitting compound may be contained in the substance. The example of such a substance other than a different light-emitting compound includes a surfactant, and the like. The state of mixing refers to a state where the nonporous or porous light-emitting material and a different light-emitting compound are physically mixed. At this point, the light-emitting material may be further mixed with another substance other than the different light-emitting compound.

The method of causing the light-emitting material which is made of the bridged organosilane of the present invention to further contain the different light-emitting compound is not particularly limited. In one of such methods, the nonporous or porous light-emitting material is mixed with a different light-emitting compound. In this case, it is possible to achieve efficient emission by dissolving the different light-emitting compound in an appropriate solvent before the mixing in order to achieve more uniform mixing.

In another method, when the light-emitting material made of the bridged organosilane of the present invention is synthesized, a different light-emitting compound is simultaneously introduced therein. Specifically, the above-described monomer is added with a different light-emitting compound and polymerized. In this case, a surfactant may be added to the reaction mixture prior to the polymerization. In the case where a surfactant is added, a porous structure is formed in the polymer by the surfactant serving as a template to form pores. However, such pores are filled with the surfactant and the different light-emitting compound, and there are substantially no pores. The amount of such a different light-emitting compound is not particularly limited. When 1 mol % to 10 mol % of a light-emitting compound is added to the monomer, it is possible to sufficiently transfer the energy of the backbone to the light-emitting compound.

In the light-emitting material, which is made of the bridged organosilane of the present invention, containing a different light-emitting compound, the backbone composed of a polymer of the bridged organosilane of the present invention can efficiently absorb light and efficiently transfer the energy to a different light-emitting compound. Accordingly, it is possible to obtain the light emission having a different wavelength based on the different light-emitting compound. In this case, the backbone composed of the polymer of the monomer serving as a light-harvesting antenna can inject the harvested light energy intensively to the different light-emitting compound. Thus, it is possible to obtain light emission with a high efficiency and a strong intensity.

The method of adsorbing, binding, filling, or mixing (hereinafter, collectively referred to as “adding” in some cases) the different light-emitting compound to the polymer obtained by polymerizing the bridged organosilane of the present invention is not particularly limited, and a commonly-used method can be adopted. For example, it is possible to adopt a method in which the polymer is sprayed with, impregnated in, or immersed in a solution containing the different light-emitting compound to be added, and then dried. In this case, the polymer may be washed as necessary. Moreover, in the process of adding or drying, the polymer may be deaerated under a reduced pressure or vacuum. In such an adding process, the different light-emitting compound is caused to be attached to the surface of the polymer, to be filled in the pores, or to be adsorbed thereto. The mechanism of the multiple color light emission is not identical among the combinations of a type and composition of the bridged organosilane and the different light-emitting compound, the distance and binding strength between these two compounds, the presence or absence of the surfactant, and the like. However, the multiple color light emission is obtainable in accordance with the combination. When the light-emitting material is produced, the different light-emitting compound added to the polymer obtained by polymerizing the bridged organosilane of the present invention can be used alone or in combination of two or more kinds.

When the light-emitting material made of the bridged organosilane of the present invention is the porous material, it is preferable that a different light-emitting compound be adsorbed (by physical adsorption and/or chemical bonding) to the porous material as described above.

When the porous material contains a different light-emitting compound adsorbed thereto, the different light-emitting compound is preferably adsorbed to the surface of the porous material, particularly to the inner wall surface of the pore. The adsorption may be a physical adsorption which occurs due to the interaction between the different light-emitting compound and a functional group existing on the surface of the porous material. Alternatively, one end of the different light-emitting compound may be fixed to the functional group existing on the surface of the porous material by chemical bonding. Note that, in the latter case, the different light-emitting compound preferably has a functional group (for example, a trialkoxysilyl group, a dialkoxysilyl group, a monoalkoxysilyl group, and a trichlorosilyl group) to be chemically bonded to a functional group existing on the surface of the porous material.

In a preferred method of adsorbing the different light-emitting compound to the porous material, the porous material is immersed in an organic solvent solution (for example, benzene and toluene) containing the different light-emitting compound dissolved therein, and the solution is stirred at a temperature of approximately 0° C. to 80° C. for approximately 1 hour to 24 hours. Thereby, the different light-emitting compound is adsorbed (fixed) to the porous material by physical adsorption and/or chemical bonding.

Such a different light-emitting compound is not particularly limited, and may be an optical functional molecule, such as porphyrins, anthracenes, an aluminum complex, a rare earth element or a complex thereof, fluorescein, rhodamine (B, 6G, and the like), coumarin, pyrene, dansyl acid, a cyanine pigment, a merocyanine pigment, a styryl pigment, and a benzstyryl pigment. Moreover, the amount of the different light-emitting compound adsorbed to the porous material is not particularly limited. In general, an amount in a range from approximately 20 parts by weight to 80 parts by weight is preferable relative to 100 parts by weight of the porous material.

Additionally, the different light-emitting compound is preferably a phosphorescent material. Such a phosphorescent material have a large difference between the adsorption wavelength and the emission wavelength when compared to a fluorescent material. Thus, the use of such phosphorescent materials allows absorption of an ultraviolet light with a short wavelength, and thereby it is possible to efficiently emit a red light with a long wavelength. When the phosphorescent material is used in combination with an organosilicon compound which emits light in an ultraviolet light region, it is possible to obtain light emission in a wide wavelength region from blue to red.

Although the polymer obtained by polymerizing the bridged organosilane of the present invention is normally in a form of particulate, the polymer can be formed into a thin film, and the thin film can be further patterned into a predetermined patterned form.

In the case where the light-emitting material in a thin-film form is obtained, firstly, the monomer is stirred in an acidic solution (for example, an aqueous solution, such as hydrochloric acid and a nitric acid, or an alcohol solution) to cause a reaction (partial hydrolysis and partial condensation reaction) to obtain a sol solution including a partial polymer of the monomer. Since the hydrolysis reaction of the monomer is likely to take place at a low pH, it is possible to accelerate the partial polymerization by reducing the pH of the system. At this point, the pH is preferably 2 or below, and more preferably 1.5 or below. Moreover, the reaction temperature can be approximately 15° C. to 40° C., and the reaction duration can be approximately 30 minutes to 90 minutes.

Subsequently, the sol solution is coated on a board with various coating methods, and thereby a thin-film light-emitting material can be produced. Note that, the coating can be conducted by using a bar coater, a roll coater, a gravure coater, or the like, in various coating methods. Moreover, dip coating, spin coating, spray coating, and the like, can also be adopted. Furthermore, it is possible to form a patterned light-emitting material on a board by coating the sol solution with an inkjet method.

Thereafter, the obtained thin film is heated to approximately 40° C. to 150° C. and dried to accelerate the condensation reaction of the partial polymer. Thereby, a three-dimensional bridged structure is preferably formed. The obtained thin film preferably has an average film thickness of 1 μm or less, and more preferably in a range from 0.1 μm to 0.5 μm. When the film thick exceeds 1 μm, the light emission efficiency due to an electric field tends to decrease.

Note that, when the above-described periodic structure is formed in the thin film, the fluorescent molecule in the thin film is formed to have the periodic structure. Thus, the emission intensity from the thin film can be further increased. Moreover, it is possible to form an ordered pore structure in the thin film by adding the above-described surfactant to the sol solution. When the thin film is a porous body, the porous body can be adsorbed to the different light-emitting compound, and thereby it is possible to obtain light emission which has a wavelength different from the original wavelength of the fluorescent molecule.

Note that such a thin-film light-emitting material can be obtained in accordance with the method described in, for example, Japanese Unexamined Patent Application Publication No. 2001-130911.

Furthermore, as the form of the polymer obtained by polymerizing the bridged organosilane of the present invention, it is possible to obtain a laminated substance which is made by lamination of nanosheets each having a thickness of 10 nm or less. To be more specific, such a layered substance can be obtained by controlling the synthesis conditions in the process of polymerization (hydrolyzation and condensation reaction) of the monomer in the presence of the surfactant.

In the case where the light-emitting material made of the bridged organosilane of the present invention is made into a layered substance, it is possible to cause the nanosheets to swell by immersing the laminated substance in a solvent. Thereby, a thin film (preferably, nanosheets each having a thickness of 10 nm or less) can be easily prepared.

Moreover, the light-emitting material made of the polymer obtained by polymerizing the bridged organosilane of the present invention may contain another compound, such as a charge-transfer material. Such charge-transfer materials include a hole-transfer material and an electron-transfer material. As the former hole-transfer material, it is possible to use, for example, hole-transfer materials of a form of polymer, such as poly(ethylene-dioxythiophene)/poly(sulfonate) [PEDOT/PSS], polyvinylcarbazole (PVK), a polyparaphenylene vinylene derivative (PPV), a polyalkylthiophene derivative (PAT), a polyparaphenylene derivative (PPP), a polyfluorene derivative (PDAF), a carbazole derivative (PVK). Meanwhile, As the latter electron-transfer material, it is possible to use an aluminum complex, oxadiazole, an oligophenylene derivative, a phenanthroline derivative, a silole compound, and the like. Note that the amount of the charge-transfer material is not particularly limited. In general, an amount in a range from approximately 0.6 parts by weight to 50 parts by weight is preferable relative to 100 parts by weight of the polymer.

When any one of such charge-transfer materials is used in combination with the thin-film light-emitting material, the charge-transfer material can be mixed with the sol solution, and then coated on a board in a thin-film form. In the combination with charge-transfer material in this manner, it is possible to obtain efficient light emission by electricity. Incidentally, in the structure of such a mixture, the polymer may be dispersed in a sea-island form in the matrix of the charge-transfer material, or the polymer and the charge-transfer material may be dispersed uniformly.

Additionally, when the charge-transfer material is used in combination with the light-emitting material which is made into a layered substance, it is possible to obtain efficient light emission by electricity upon separating the nanosheets which form the layered substance, and dispersing the nanosheets into the charge-transfer material.

Furthermore, in the case where the charge-transfer material is used in combination with the particulate light-emitting material, it is possible to obtain efficient light emission by electricity upon dispersing the particles into the charge-transfer material. Note that, the average particle diameter of the particulate light-emitting material is preferably 1 μm or less, and more preferably in a range of 100 nm or less where no light scattering occurs.

### EXAMPLES

Hereinafter, the present invention will be more specifically described on the basis of Examples and Comparative examples. However, the present invention is not limited to the Examples described below.

### Example 1

### synthesis of 2,7-Bis(triethoxysilyl)fluorene)

A mixture of 3 g (9.3 mmol) of 2,7-dibromofluorene, 159 mg (0.42 mmol, 4.5 mol %) of [Rh(CH₃CN)₂(cod)]BF₄ and 6.84 g (18.5 mmol, 2 eq.) of n-Bu₄NI was added with 90 mL of dimethylformamide (DMF) and 7.74 ml (55.5 mmol, 6 eq.) of triethanolamine (TEA) under a nitrogen atmosphere to obtain a mixed solution. Then, 5.55 ml (30.0 mmol, 3.2 eq.) of triethoxysilane [(EtO)₃SiH] was added dropwise to the mixed solution under a temperature condition of 0° C. to obtain a suspension. Subsequently, the suspension thus obtained was stirred under a nitrogen atmosphere and a temperature condition of 80° C. for 2 hours. Thereafter, the solvent was removed by distillation with a vacuum pump, and a residue was extracted with ether. After that, a salt thus formed was removed by filtering with celite. The solvent was removed by distillation from the organic phase with an evaporator to obtain a crude product. The crude product thus obtained was dissolved in 120 ml of ether, and then purified by filtration with through activated carbon (Kiriyama funnel, diameter: 5 cm, thickness: 1.5 mm). Thereby, a fluorene-silane compound was obtained (a colorless, transparent, syrupy liquid: a yield of 2.34 g and 51%).

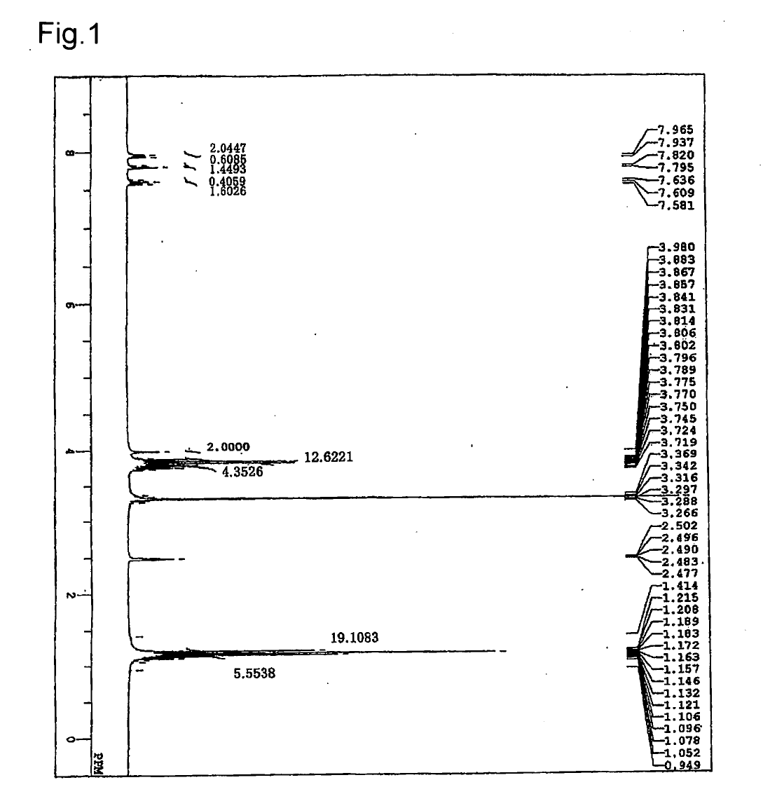

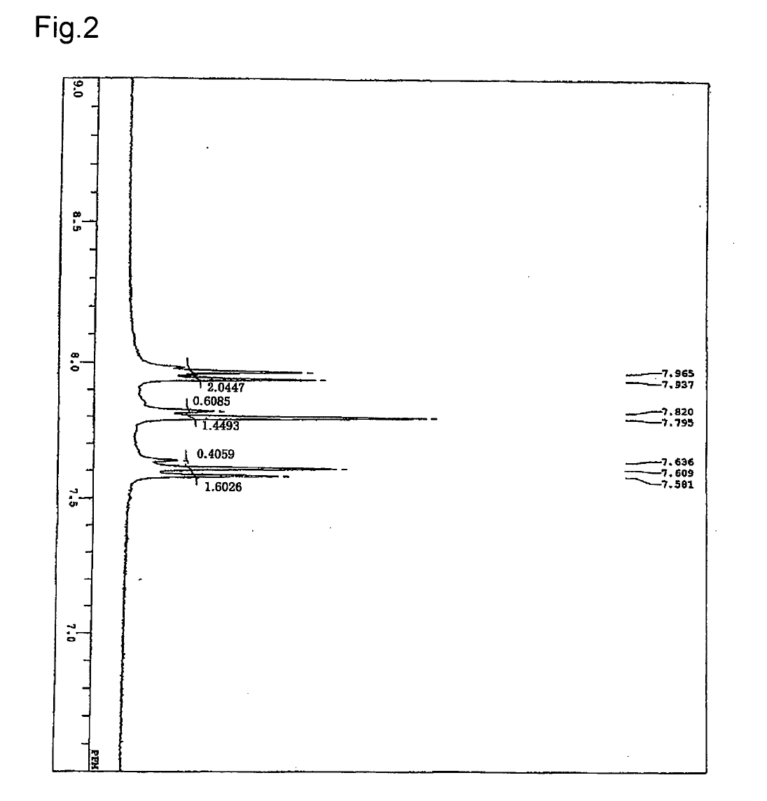

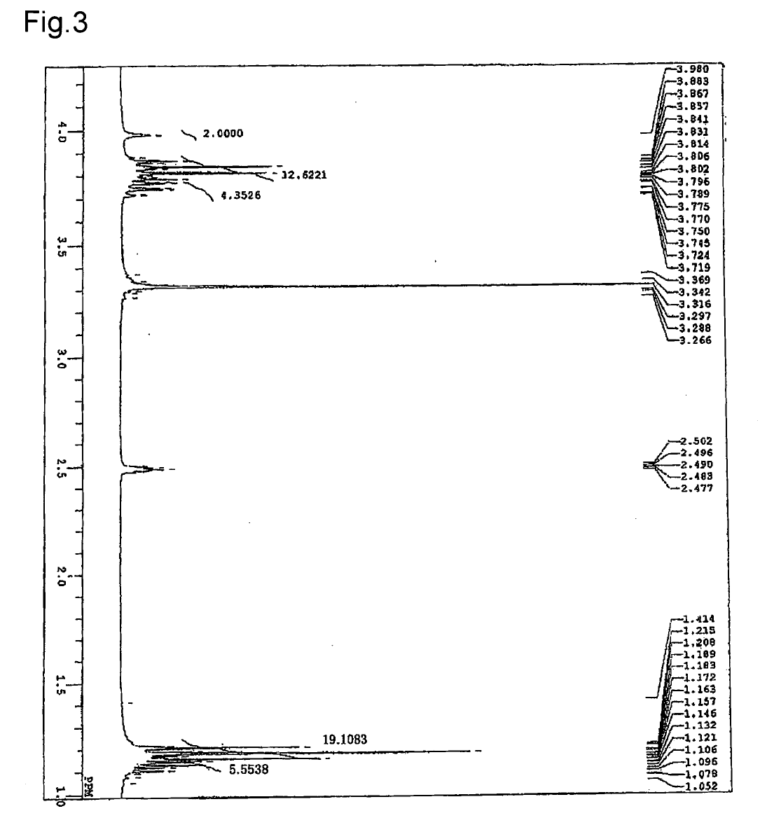

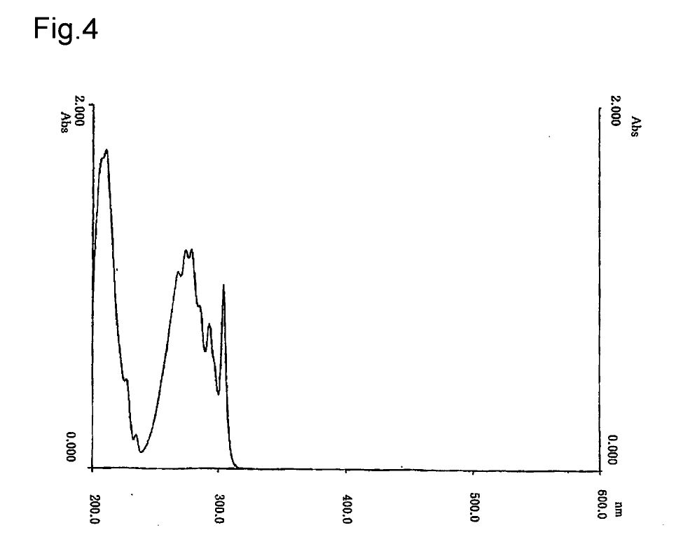

The obtained fluorene-silane compound was subjected to ¹H NMR measurement. The obtained results are shown in FIGS. 1 to 3 and below. Moreover, the UV spectrum of the obtained fluorene-silane compound is shown in FIG. 4.

¹H NMR (DMSO) δ7.94 (d, J=7.56 Hz, 2H), 7.80 (s, 2H), 7.59 (d, J=7.56 Hz, 2H), 3.98 (s, 2H), 3.82 (q, J=6.75 Hz, 12H), 1.18 (t, J=7.02 Hz, 18H).

Based on the NMR measurement results, it was confirmed that the fluorene-silane compound obtained in Example 1 was a fluorene-disilane compound expressed by the following general formula (86).

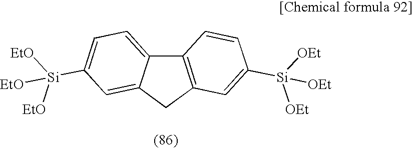

### Example 2

### synthesis of 1.6-Bis(diallylethoxysilyl)pyrene)

A mixture of 3.57 g (9.90 mmol) of 1,6-dibromopyrene, 226 mg (0.594 mmol, 6 mol %) of [Rh(CH₃CN)₂(cod)]BF₄ and 21.94 g (59.4 mmol, 6 eq.) of tetrabutylammoniumiodide was added with 300 mL of DMF under a nitrogen atmosphere to obtain a mixed solution. Then, after 8.28 ml (59.4 mmol, 6 eq.) of triethylamine was added to the mixed solution, 7.31 ml (39.6 mmol, 4 eq.) of triethoxysilane was further added dropwise under a temperature condition of 0° C. to obtain a suspension. Subsequently, the suspension thus obtained was stirred under a nitrogen atmosphere and a temperature condition of 80° C. for 45 minutes. Thereafter, after the DMF in the obtained suspension was removed with a vacuum pump, the suspension was extracted with ether three times, filtered with celite, and concentrated to obtain a crude product (I) (a yield of 4.48 g).

Then, since the obtained crude product contained pyrene, triethoxysilyl pyrene, and 1,6-bistriethoxysilyl pyrene, the crude product was allylated to purify by silica gel chromatography. Specifically, 51.8 ml (51.8 mmol) of an allylmagnesium bromide solution (1.0 M in diethyl ether) was added dropwise to 3.00 g of the crude product (I) under a nitrogen atmosphere and a temperature condition of 0° C. to obtain a mixture. Subsequently, the mixture thus obtained was stirred at room temperature (25° C.) for 3 days, and cooled to 0° C. The pH of the mixture was adjusted to 7 with 10% HCl. The mixture was then washed with sodium acid carbonate and sodium chloride independently, dried with anhydrous magnesium sulfate, filtered, and concentrated to obtain a crude product (II) (a yield of 2.3 g). The crude product (II) thus obtained by the allylation was separated and purified by silica gel chromatography (eluent, hexane:benzene=7:1). Thereby, a pyrene-silane compound was obtained (a yellow, crystalline solid: a yield of 415 g and 9.2%).

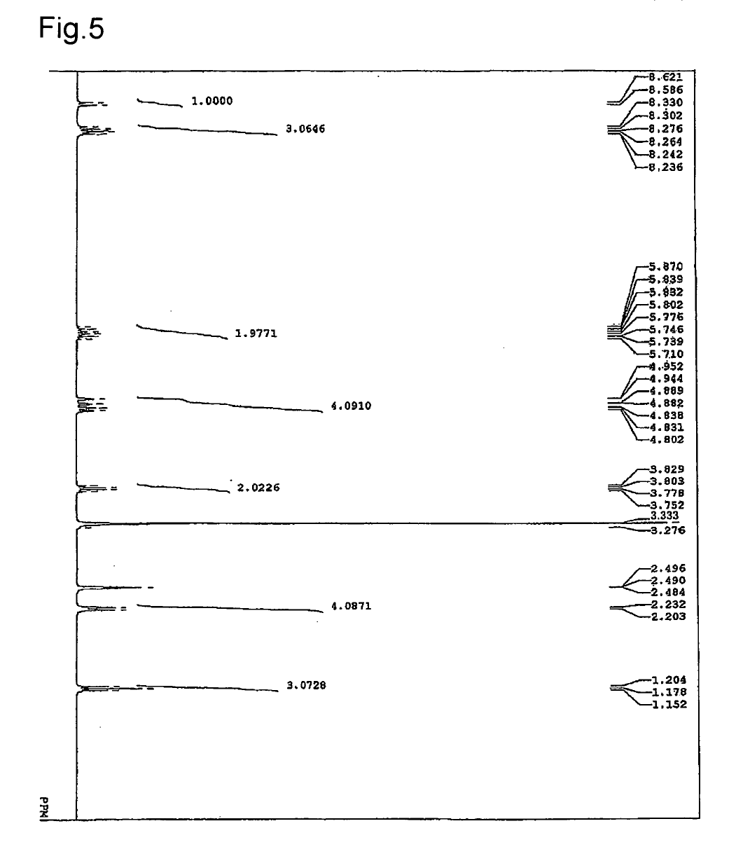

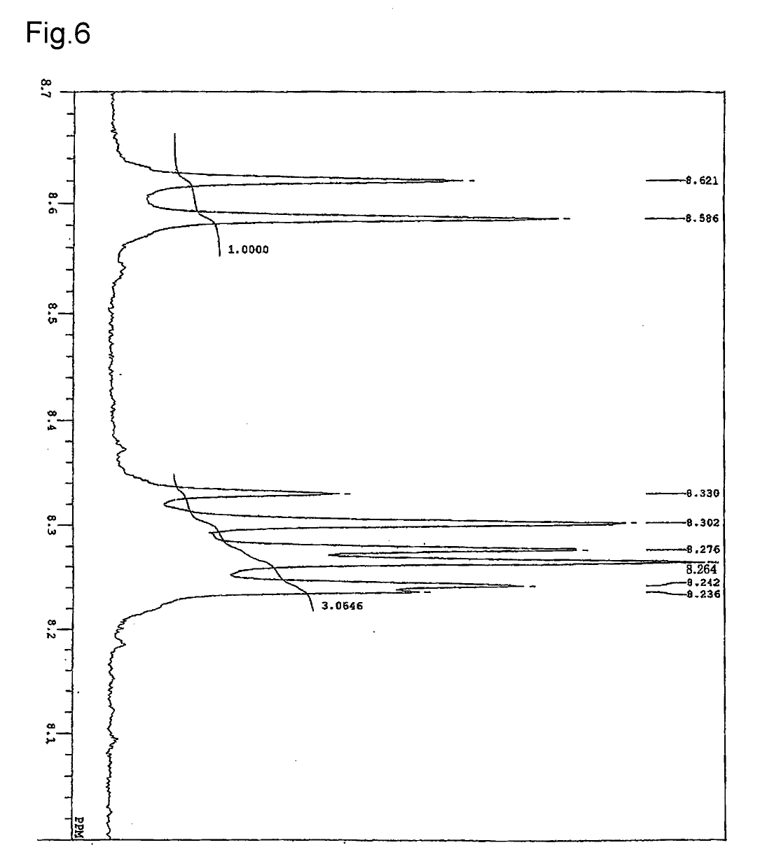

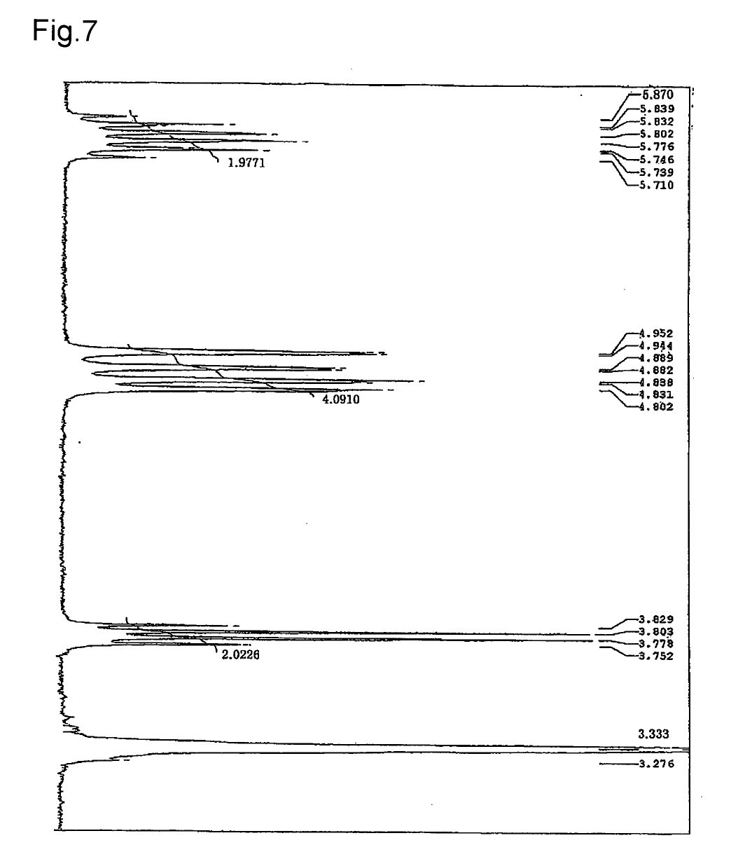

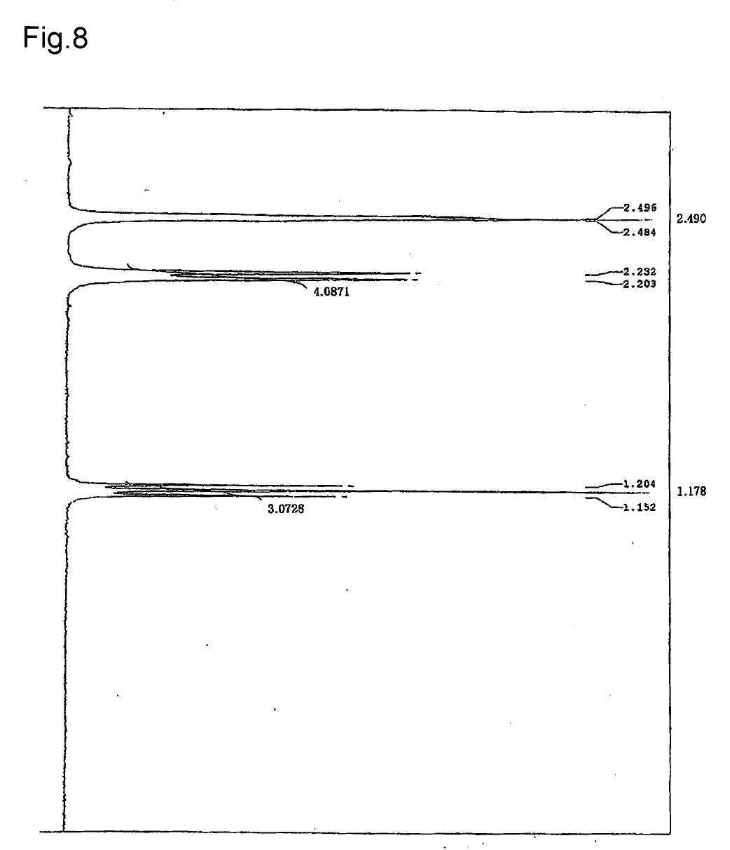

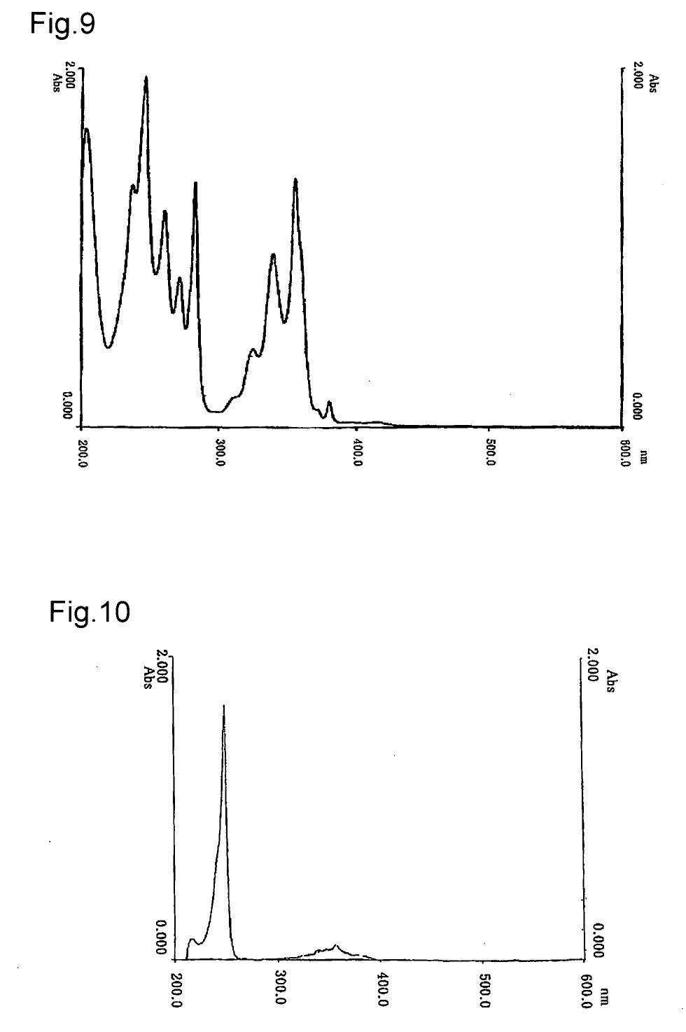

The obtained pyrene-disilane compound was subjected to ¹H NMR measurement. The obtained results are shown in FIGS. 5 to 8 and below. Moreover, the UV spectrum of the obtained pyrene-silane compound is shown in FIG. 9.

¹H NMR (DMSO) δ8.63 (d, J=9.45 Hz, 2H), 8.33-8.24 (m, 6H), 5.87-5.71 (m, 4H), 4.92 (d, J=17.0 Hz, 4H), 4.82 (d, J=8.91 Hz, 4H), 3.79 (q, J=7.02 Hz, 4H), 2.22 (d, J=7.83 Hz, 8H), 1.18 (t, J=7.02 Hz, 6H).

Based on the NMR measurement results, it was confirmed that the pyrene-silane compound obtained in Example 2 was a pyrene-disilane compound expressed by the following general formula (87).

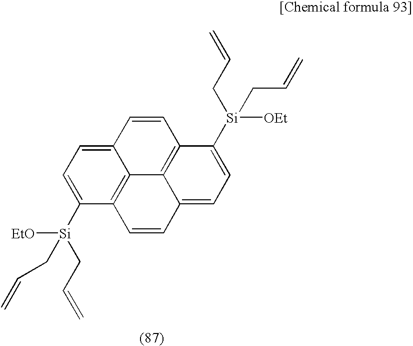

### Example 3

### synthesis of 2.7-Bis(triethoxysilyl)acridine)

### Synthesis of Benzyltriethylammonium Tribromide (BTEABr₃)

In an open system, 22.8 g (100 mmol) of benzyltriethylammonium chloride and 7.6 g (50 mmol) of sodium bromide were added with 160 ml of ion-exchanged water, and stirred until the compounds were dissolved. Then, 100 ml of dichloromethane was added thereto, and the resultant mixture was vigorously stirred to mix the aqueous phase and organic phase. Subsequently, the mixture was cooled to 0° C., and added dropwise with 40.8 ml (350 mmol) of 47% hydrobromic acid in 15 minutes using a dropping funnel. After the resultant was stirred, the organic phase and the aqueous phase were separated, and the aqueous phase was extracted three times with 40 ml of dichloromethane. Thereafter, the organic phase thus obtained was dried with anhydrous magnesium sulfate, and concentrated to recrystallize the residual solid by using a solvent of dichloromethane and diethyl ether with a volumetric ratio of 5:1. Thereby, BTEABr₃ was obtained (an orange crystal: a yield of 37.1 g and 81%).

### Synthesis of 2.7-Dibromoacridine

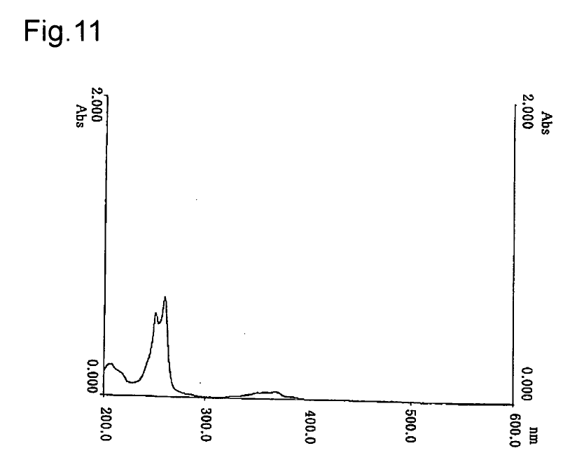

6.29 g (35.1 mmol) of acridine and 31.6 g (70.2 mmol, 2 eq.) of the BTEABr₃ obtained as described above were added with 700 ml of methanol, and refluxed under a temperature condition of 80° C. for 2 hours. After that, the mixture was cooled to room temperature (25° C.) and filtered. Half of the filtrate thus obtained was concentrated to obtain a precipitate. The precipitate was separated by filtration, and thoroughly washed with ethanol to obtain 2,7-dibromoacridine (yellow solid: a yield of 6.81 g and 63%) expressed by the general formula (75). The UV spectra of acridine and 2,7-dibromoacridine thus obtained are shown in FIGS. 10 and 11, respectively.

The obtained 2,7-dibromoacridine was subjected to ¹H NMR measurement, and the obtained result is shown below.

¹H NMR (DMSO) δ9.10 (s, 1H), 8.52 (s, 2H), 8.11 (d, J=9.32 Hz, 2H), 7.99 (d, J=9.32 Hz, 2H).

### Synthesis of 2.7-Bis(triethoxysilyl)acridine

Under a nitrogen atmosphere, a mixture of 4.1 g (13.4 mmol) of 2,7-dibromoacridine, 304 mg (0.801 mmol, 6 mol %) of [Rh(CH₃CN)₂(cod)]BF₄, and 9.90 g (26.8 mmol, 2 eq.) of tetrabutylammoniumiodide was added with 160 mL of dimethylformamide (DMF) to obtain a mixed solution. Then, the mixed solution was added with 5.60 ml (40.2 mmol, 3 eq.) of triethylamine, and then was added dropwise with 4.95 ml (26.8 mmol, 2 eq.) of triethoxysilane under a temperature condition of 0° C. to obtain a suspension. Thereafter, the suspension thus obtained was stirred under a nitrogen atmosphere and a temperature condition of 80° C. for 2 hours. After the stirring, the DMF was removed with a vacuum pump, and the suspension was extracted with ether three times, filtered with celite, and concentrated to obtain a crude product (a yield of 4.78 g). The crude product thus obtained was dissolved in 120 ml of ether, and then purified by filtering the resultant through activated carbon (Kiriyama funnel, diameter: 5 cm, thickness: 1.5 cm). Thereby, an acridine-silane compound was obtained (a red oily form: a yield of 3.44 g and 51%).

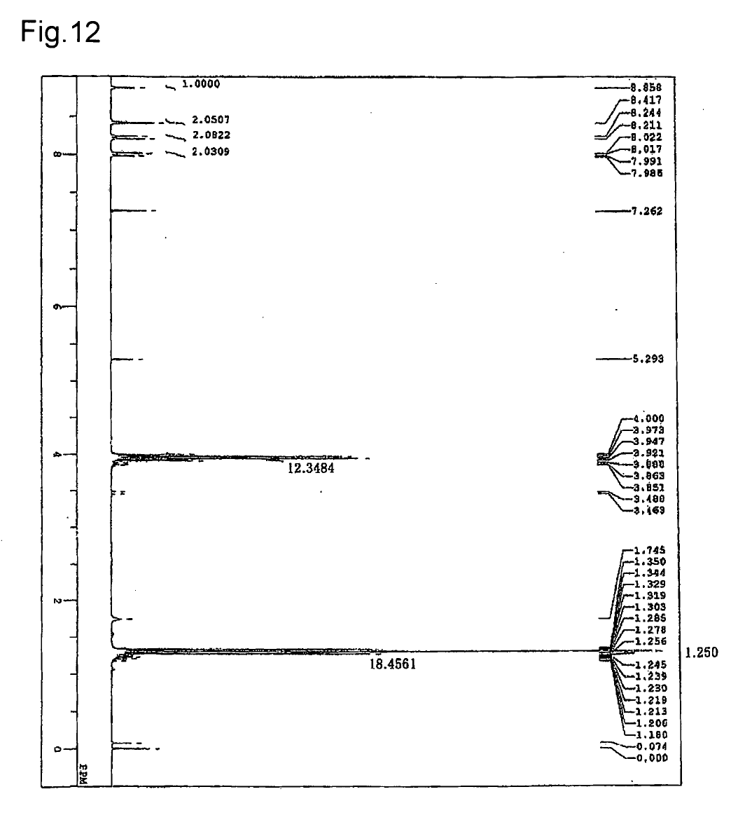

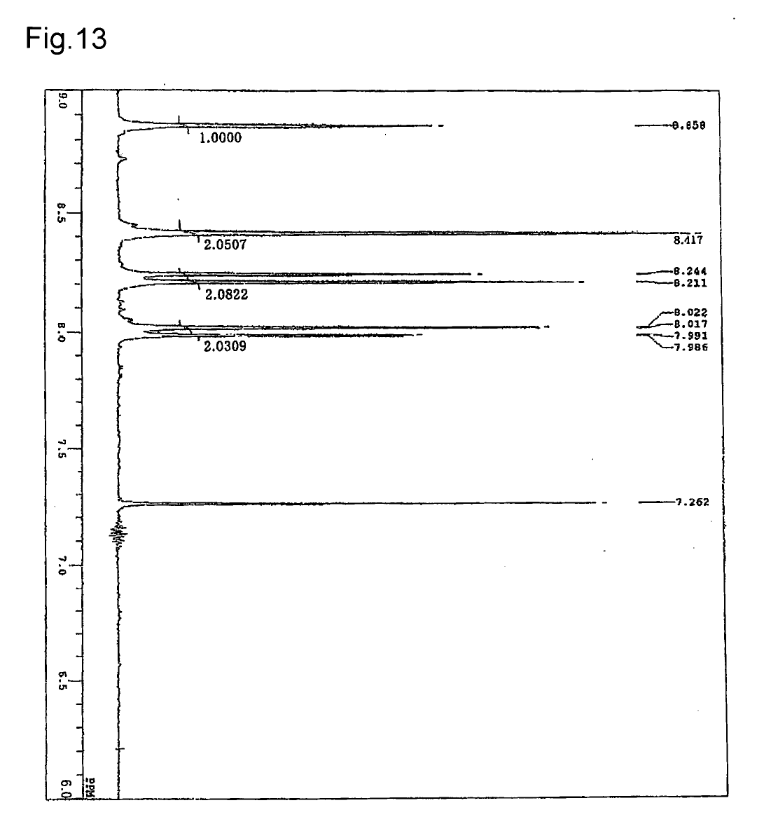

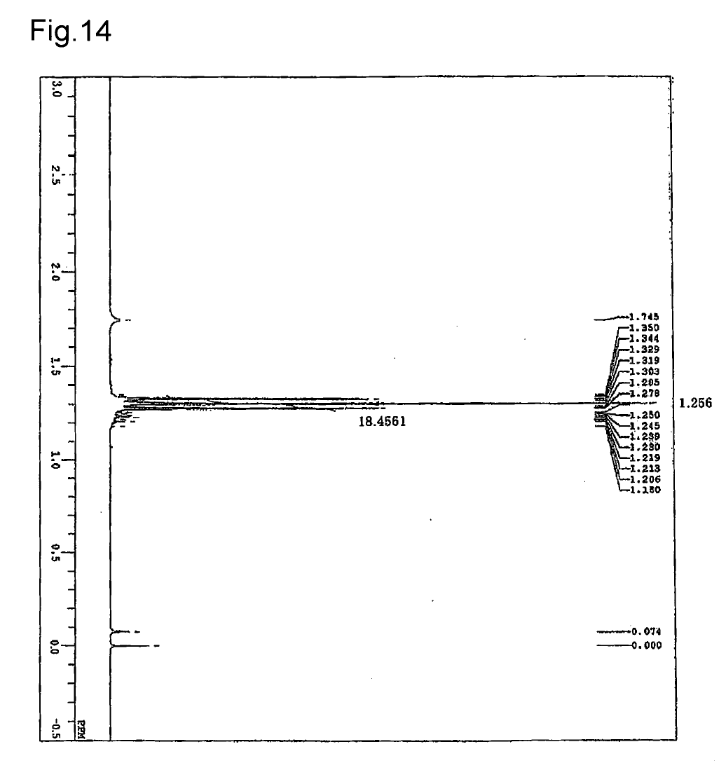

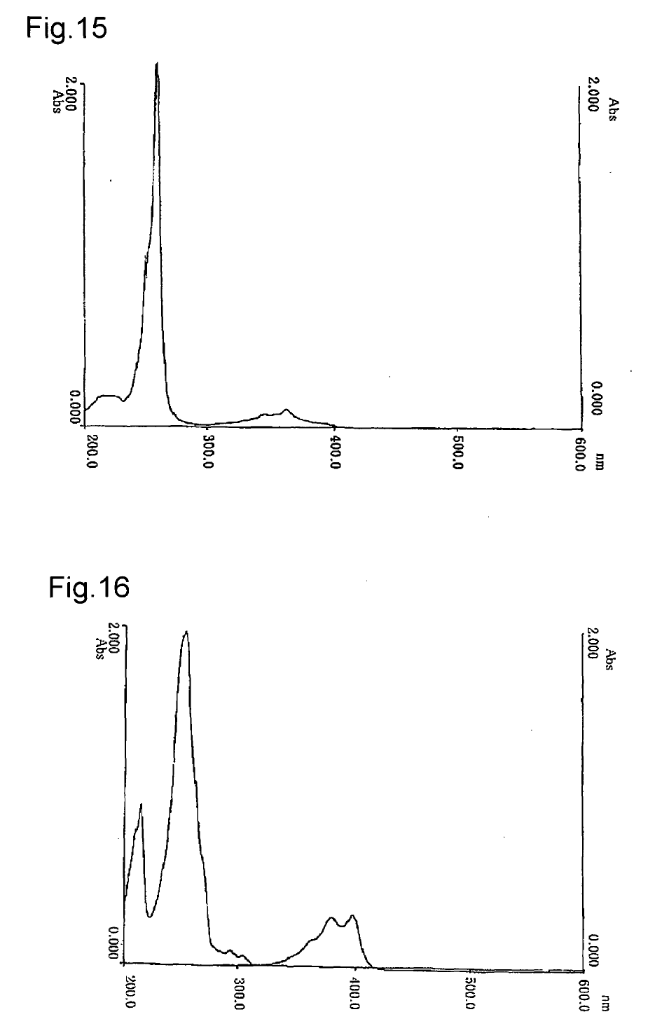

The obtained acridine-silane compound was subjected to ¹H NMR measurement. The obtained results are shown in FIGS. 12 to 14 and below. Moreover, the UV spectrum of the obtained acridine-silane compound is shown in FIG. 15.

¹H NMR (CDCL₃) δ8.86 (s, 1H), 8.42 (s, 2H), 8.23 (d, J=8.64 Hz, 2H), 8.00 (d, J=8.64 Hz, 2H), 3.96 (q, J=7.02 Hz, 12H), 1.30 (t, J=7.02 Hz, 18H).

Based on the NMR measurement results, it was confirmed that the acridine-silane compound obtained in Example 3 was an acridine-disilane compound expressed by the following general formula (88).

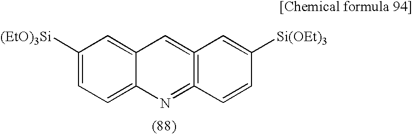

### Example 4

### synthesis of 2.7-Bis(triethoxysilyl)acridone)

### Synthesis of 2.7-Dibromoacridone

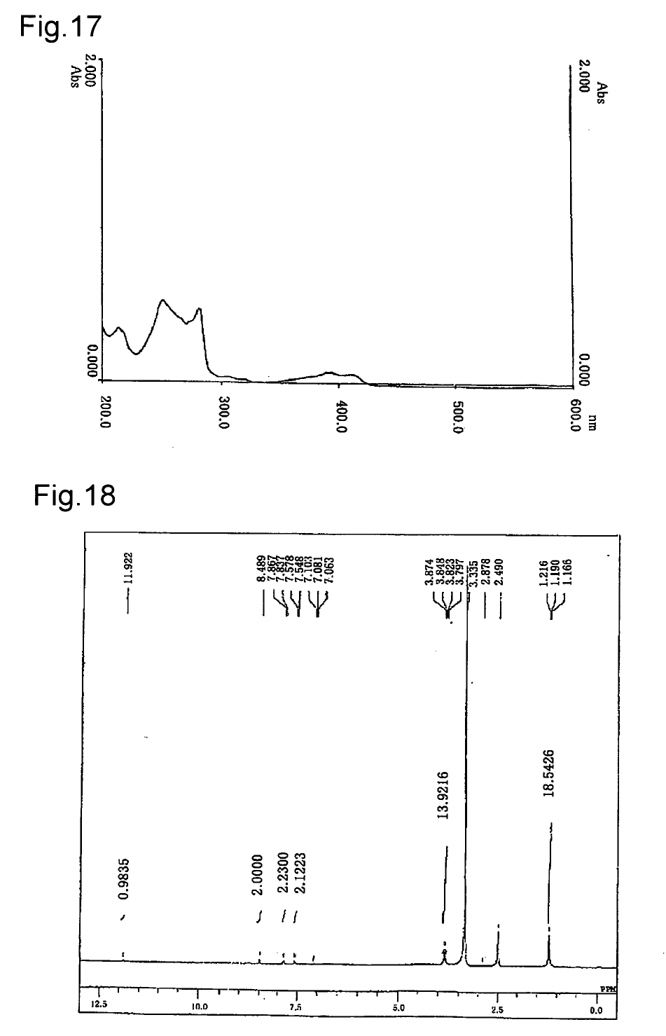

A mixture of 1.95 g (10 mmol) of acridone and 9.0 g (20 mmol, 2 eq.) of BTEABr₃ obtained as in Example 3 was added with 500 ml of acetic acid, and stirred under a temperature condition of 80° C. for 8 hours. Then, the mixture was filtered without a cooling process, and a precipitate was collected to obtain 2,7-dibromoacridone (a yellow solid: a yield of 2.2 g and 61%) expressed by the general formula (79). The UV spectra of acridone and 2,7-dibromoacridone thus obtained are shown in FIGS. 16 and 17, respectively. Moreover, the 2,7-dibromoacridone thus obtained was subjected to ¹H NMR measurement, and the obtained result is shown below.

¹H NMR (DMSO) δ12.09 (s, 1H), 8.27 (s, 2H), 7.88 (d, J=2.43 Hz, 2H), 7.52 (d, J=2.43 Hz, 2H).

### Synthesis of 2.7-Bis(triethoxysilyl)acridone

Under a nitrogen atmosphere, a mixture of 2.03 g (˜5.75 mmol) of 2,7-dibromoacridone, 131 mg (0.345 mmol, 6 mol %) of (Rh(CH₃CN)₂(cod)]BF₄, and 4.25 g (11.5 mmol, 2 eq.) of tetrabutylammonium bromide was added with 80 mL of dimethylformamide (DMF) to obtain a mixed solution. Then, the mixed solution was added with 4.81 ml (34.5 mmol, 6 eq.) of triethylamine. Subsequently, 4.25 ml (23.0 mmol, 4 eq.) of triethoxysilane was added dropwise under a temperature condition of 0° C. to obtain a suspension. Thereafter, the suspension thus obtained was stirred under a nitrogen atmosphere and a temperature condition of 80° C. for 2 hours. After the stirring, the DMF was removed with a vacuum pump, and then the suspension was extracted with ether three times, filtered with celite, and concentrated to obtain a crude product (a yield of 3.1 g). Then, the crude product thus obtained was dissolved in 120 ml of ether, and purified by filtering the resultant through activated carbon (Kiriyama funnel, diameter: 5 cm, thickness: 1.5 cm). Thereby, an acridone-silane compound was obtained (a yellow solid: a yield of 674 mg and 23%).

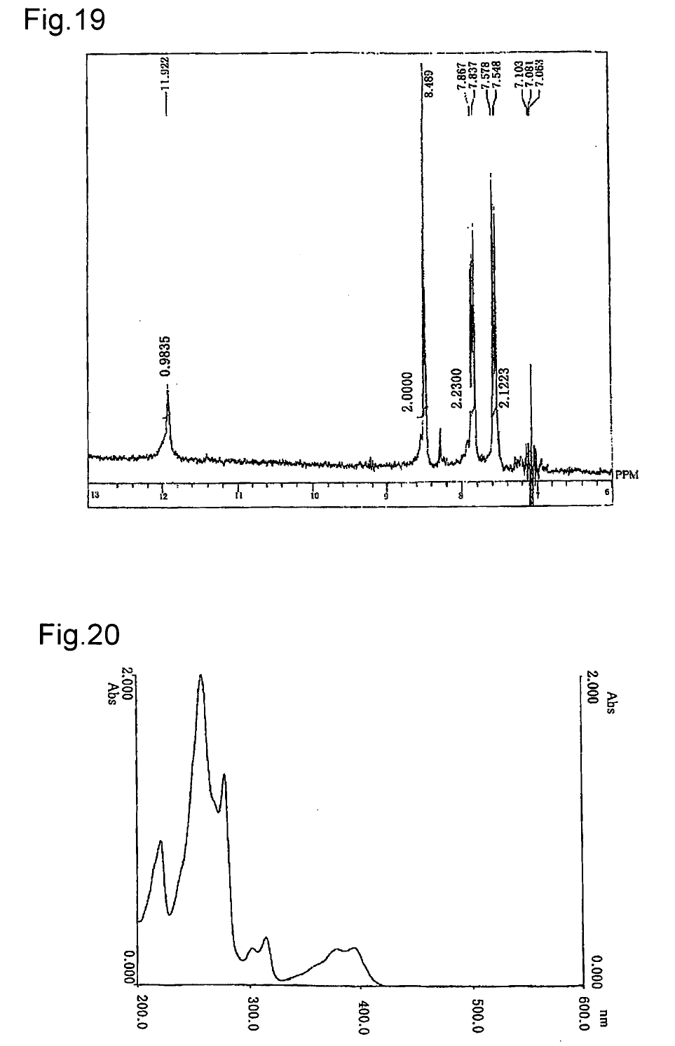

The acridone-silane compound thus obtained was subjected to ¹H NMR measurement. The obtained results are shown in FIGS. 18 and 19 and below. Moreover, the UV spectrum of the obtained acridone-silane compound is shown in FIG. 20.

¹H NMR (CDCL₃) δ11.92 (s, 1H), 8.49 (s, 2H), 7.85 (d, J=8.10 Hz, 2H), 7.63 (d, J=8.10 Hz, 2H), 3.84 (q, J=7.02 Hz, 12H), 1.19 (t, J=7.02 Hz, 18H).

Based on the NMR measurement results, it was confirmed that the acridone-silane compound obtained in Example 4 was an acridone-disilane compound expressed by the following general formula (89).

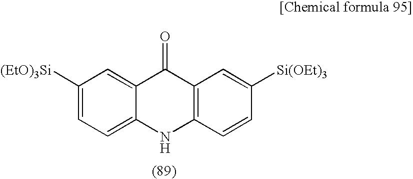

### Example 5

### synthesis of 4,4′″-Bis(triethoxysilyl)quaterphenyl)

### Synthesis of 4,4′″-diiodoquaterphenyl

4,4′″-bis(triethoxysilyl)quaterphenyl was prepared by a sililation reaction with a Rh catalyst performed on 4,4′″-diiodoquaterphenyl. In the purification of the ethoxysilane compound, column chromatography filled with silica gel 60 silanized (Merck; 0.063 mm to 0.200 mm) was used. A diiodo compound to serve as a precursor was synthesized with a method reported by Novikov et al. (a method shown in the following reaction formulae (A) to (C)). Incidentally, the sililation reaction with the Rh catalyst performed on 4,4′″-dibromoquaterphenyl hardly progressed

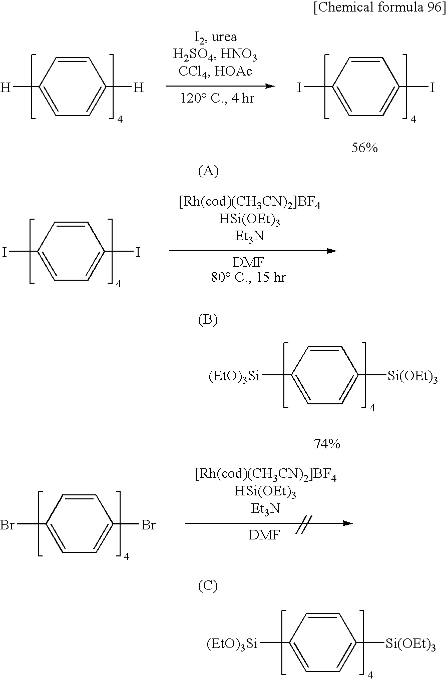

### Synthesis of 4,4′″-diiodoquaterphenyl

A stirrer was put into a 200 ml three-necked flask, and a dropping funnel with a pressure-equalizing side tube, a reflux condenser, and a nitrogen-gas inlet were attached to the flask. Into the flask, 3.0 g (9.8 mmol) of p-quaterphenyl (available from Sigma-Aldrich Corp.), 3.0 g (49.9 mmol) of urea, 45 mL of acetic acid (available from Wako Pure Chemical Industries, Ltd.), and 6 mL of carbon tetrachloride (available from Wako Pure Chemical Industries, Ltd.) were added. While stirring the mixture of the flask, 9.96 g (39.2 mmol) of Iodine was added thereto at once, and a suspension was obtained.

The dark red suspension thus obtained was heated to 120° C. in an oil bath. Then, while stirring the suspension thoroughly, a mixed acid made up of 9.0 ml of concentrated sulfuric acid (available from Wako Pure Chemical Industries, Ltd.) and 2.4 ml of concentrated nitric acid (available from Nacalai Tesque Inc.) was added dropwise to the suspension using the dropping funnel for one hour. After the dropping was finished, the suspension was further stirred for 4 hours under a temperature condition of 120° C. Upon the completion of the stirring process, a dark violet solution was obtained. Subsequently, the solution thus obtained was cooled down to room temperature (25° C.), and then added with 200 mL of pure water to dilute the solution. After the dilution, a brown suspension was obtained.

Then, a solid matter was precipitated from the brown suspension obtained as described above with a centrifuge (3600 rpm, 5 min), and a supernatant was carefully removed using a pipette. Subsequently, the precipitate thus obtained was washed with pure water, and separated again by centrifugation. This process was repeated three times. Thereafter, the resultant was washed with methylene chloride three times, and subsequently washed with ether three times. A yellowish powder thus obtained was recrystallized from cyclohexane, and thereby 4,4′″-diiodoquaterphenyl was obtained (a yield of 3.0 g and 56%). The following reaction formula (D) shows an outline of the synthesis method for the 4,4′″-diiodoquaterphenyl.

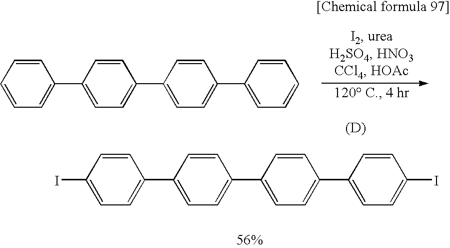

### Synthesis of 4,4′″-Bis(triethoxysilyl)quaterphenyl

A stirrer was put into a 200 ml three-necked flask, and a reflux condenser, a septum cap, and a nitrogen-gas inlet were attached to the flask. Into the flask, 500 mg (0.89 mmol) of the 4,4′″-diiodoquaterphenyl as obtained above, 0.74 ml (5.3 mmol) of triethylamine, and 50 ml of DMF were added. Then, the mixture was bubbled with a nitrogen gas for 30 minutes while being stirred. Subsequently, the mixture was added with 13 mg (0.036 mmol) of [Rh(CH₃CN)₂(cod)]BF₄ and 0.66 ml (3.56 mmol) of triethoxysilane, and stirred at a temperature condition of 80° C. for 15 hours. Thereafter, the temperature was decreased to room temperature (25° C.), and then a gray suspension thus obtained was filtered under a nitrogen atmosphere. A filtrate thus obtained was concentrated, and thereby a yellow solid was obtained. The yellow solid was purified by flash chromatography (developing solvent: dry hexane) filled with reversed phase silica gel (Merck; silica gel 60 silanized (0.063 mm to 0.200 mm) for column chromatography was used). Thereby, a quaterphenyl-silane compound was obtained (a white solid: a yield of 410 mg and 74%). The following reaction formula (E) shows an outline of the synthesis method for the quaterphenyl-silane compound.

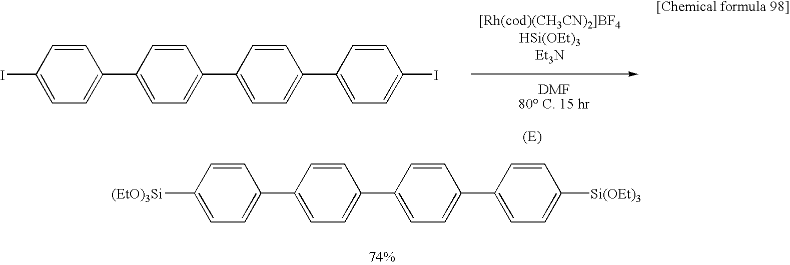

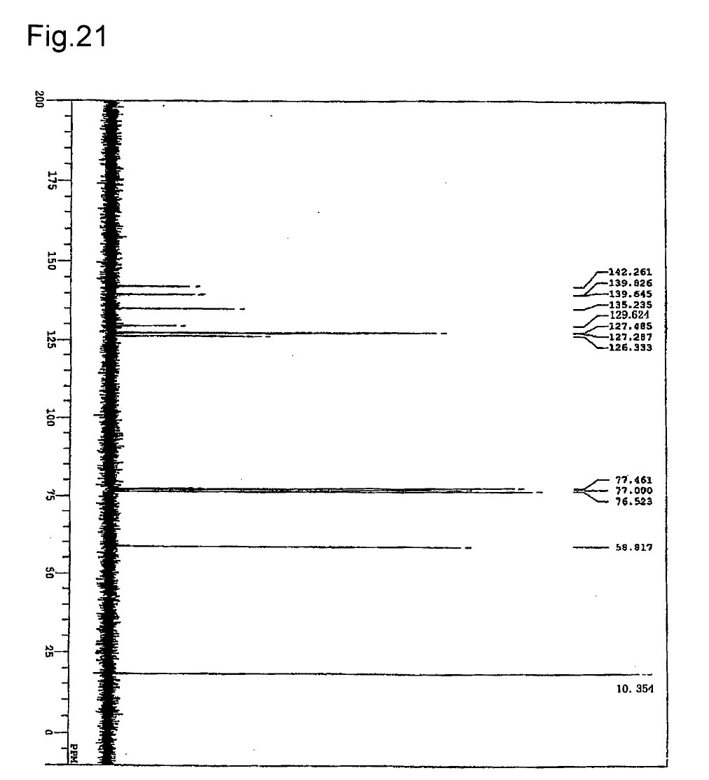

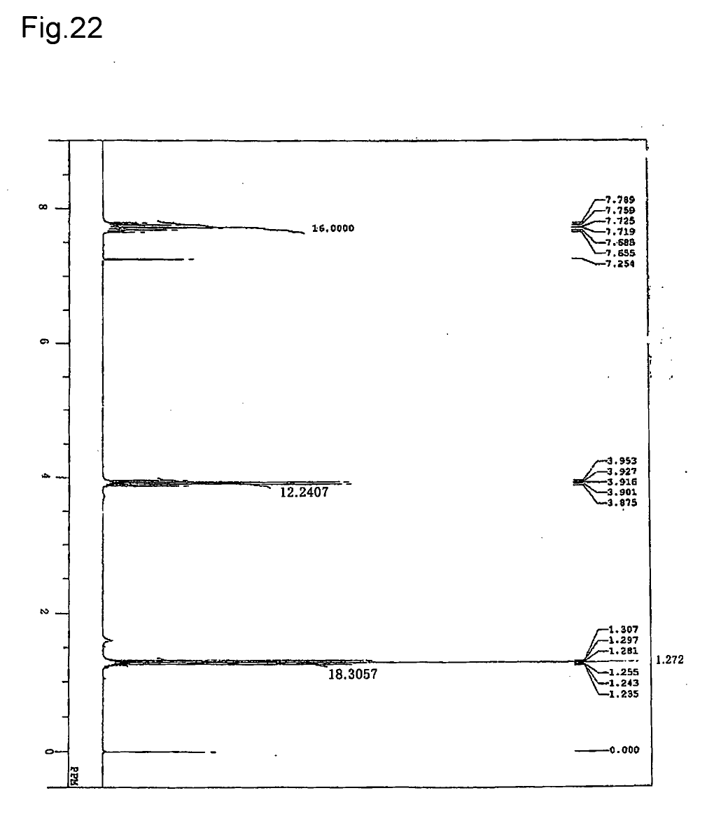

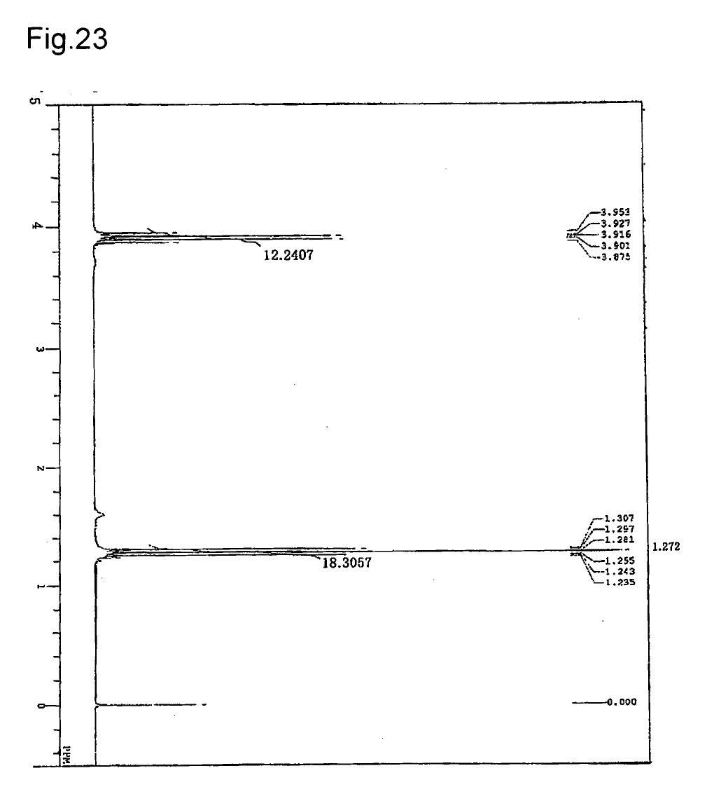

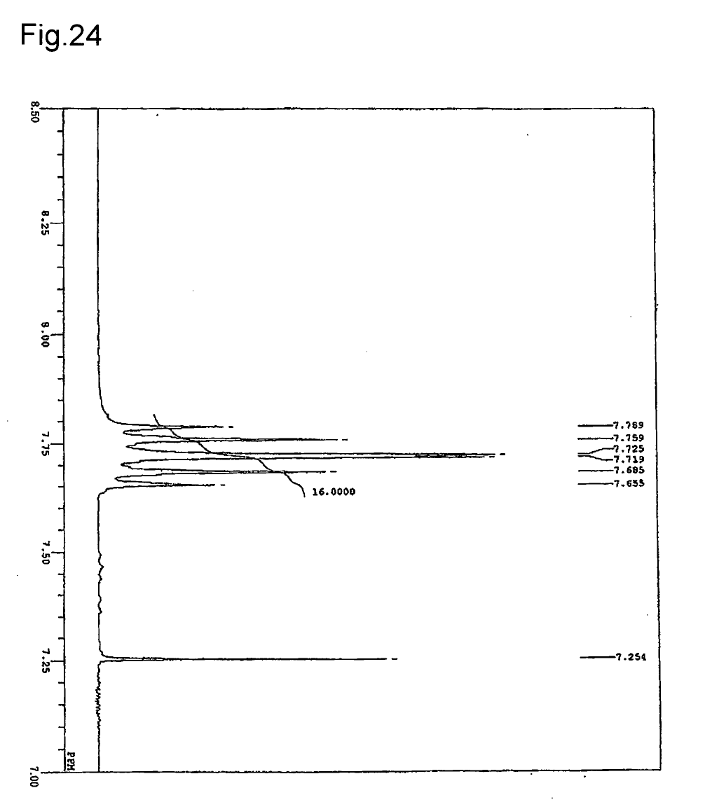

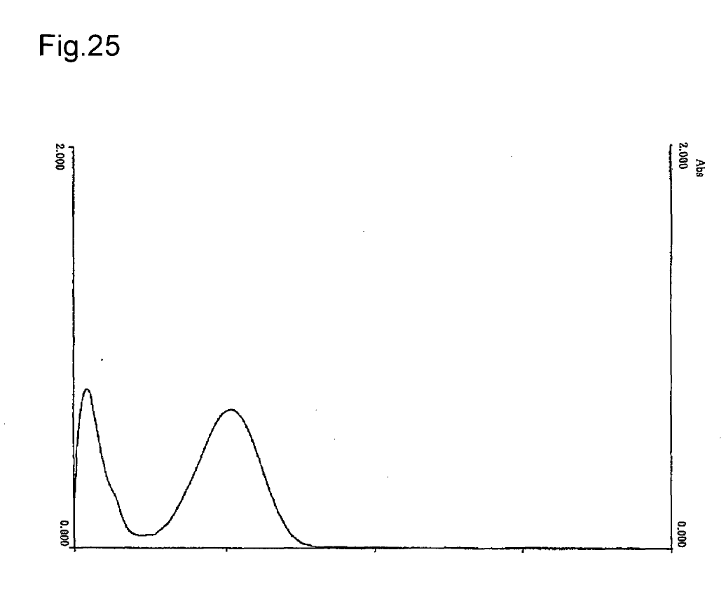

The quaterphenyl-silane compound thus obtained was subjected to NMR measurement. The measurement results are shown below. Among the obtained results, FIG. 21 shows a graph of ¹³C-NMR, and FIGS. 22 to 24 show graphs of ¹H-NMR. Moreover, the UV spectrum of the obtained quaterphenyl-disilane compound is shown in FIG. 25.

¹H-NMR (500 MHz, CDCl₃) 1.26 (t, J=7.5 Hz, 18H), 3.89 (q, J=7.5 Hz, 12H), 7.65 (d, J=8.0 Hz, 4H), 7.70 (dd, J=8.0, 8.0 Hz, 4H), 7.71 (dd, J=8.0, 7.5 Hz, 4H), 7.76 (d, J=7.5 Hz, 4H); ¹³C-NMR (125 MHz, CDCl₃) 18.2, 58.7, 126.3, 127.2, 127.4, 129.7, 135.2, 139.6, 139.8, 142.2; ²⁹SI-NMR (99 MHz, CDCl₃) −57.0; FAB HRMS (NBA) m/z 630.2836, calcd for C₃₆H₄₆O₆Si₂ 630.2833.

Based on the NMR measurement results, it was confirmed that the quaterphenyl-silane compound obtained in Example 5 was a quaterphenyl-disilane compound expressed by the following general formula (90).

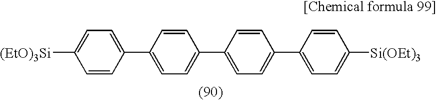

### Example 6

### synthesis of 2,6-Bis(triethoxysilyl)anthracene)

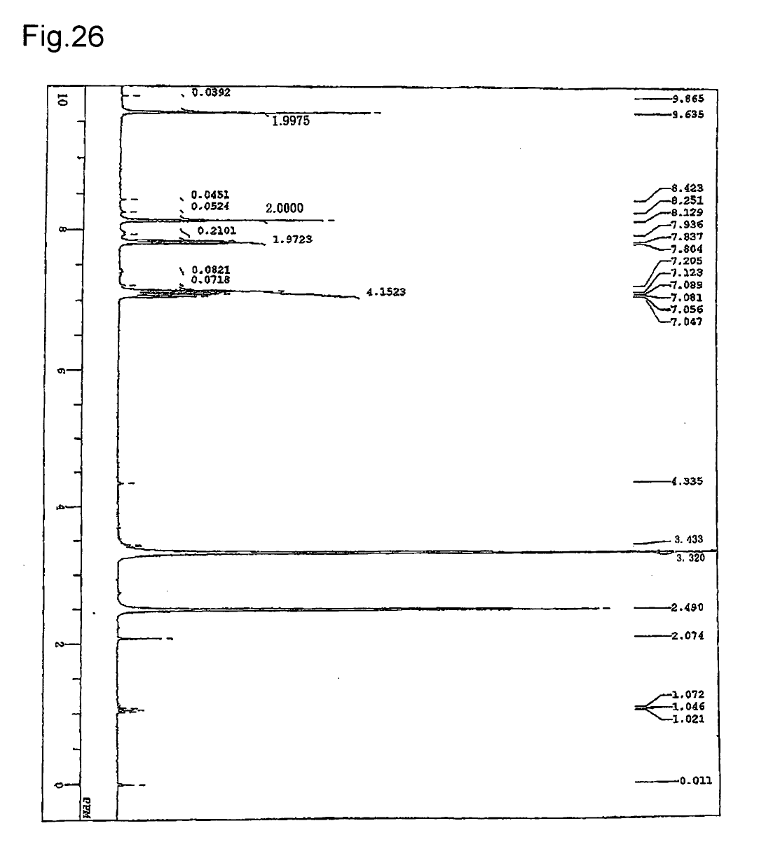

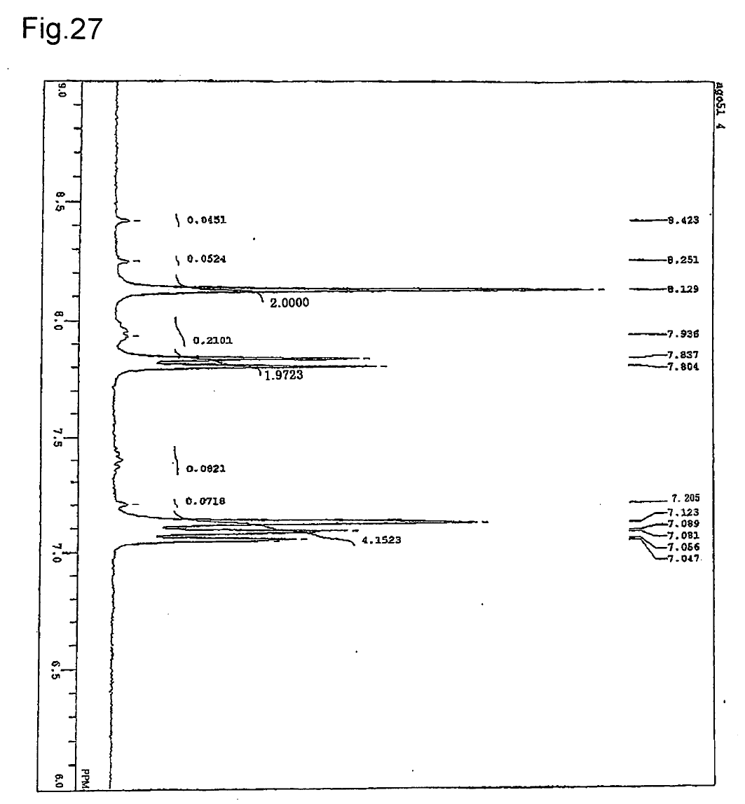

Into a two-necked flask containing a 5 mm-square aluminum plate (9.21 g, 341.4 mmol) therein, 82 ml of 1.5% of HgCl₂ solution prepared in advance was added, and stirred for 30 seconds. After the stirring, 24.6 ml of distilled water, 16.4 ml of ethanol, and 16.4 ml of concentrated ammonia water were sequentially added. Then, 4.1 g (17.1 mmol) of 2,6-dihydroxyanthracene-9.10-dione (anthraflavic acid) was further added thereto under a nitrogen flow, and the mixture was stirred under a temperature condition of 63° C. The reaction was traced with silica gel thin-layer chromatography (TLC). After the completion of the reaction, the mixture was left to cool to room temperature (25° C.), and an amalgam was removed by filtration. The filtrate thus obtained was added with concentrated hydrochloric acid to adjust the pH to 1, and then the pH was further adjusted to 4 with a saturated sodium acid carbonate solution. After the pH was adjusted in this manner, the resultant was concentrated, dissolved in acetone, and filtered with celite. Thereafter, the obtained filtrate was concentrated to obtain a reaction product, and the reaction product was recrystallized with hot ethanol. Thereby, 2,6-dihydroxyanthracene was obtained (a yield of 1.9 g and 53%). The following reaction formula (F) shows an outline of the synthesis method for the 2,6-dihydroxyanthracene. Moreover, the NMR measurement results of the 2,6-dihydroxyanthracene are shown in FIGS. 26 and 27.

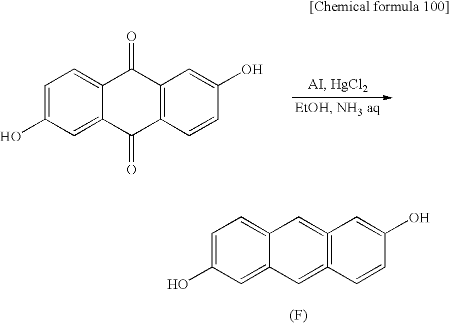

### Synthesis of 2,6-dihydroxyanthracene

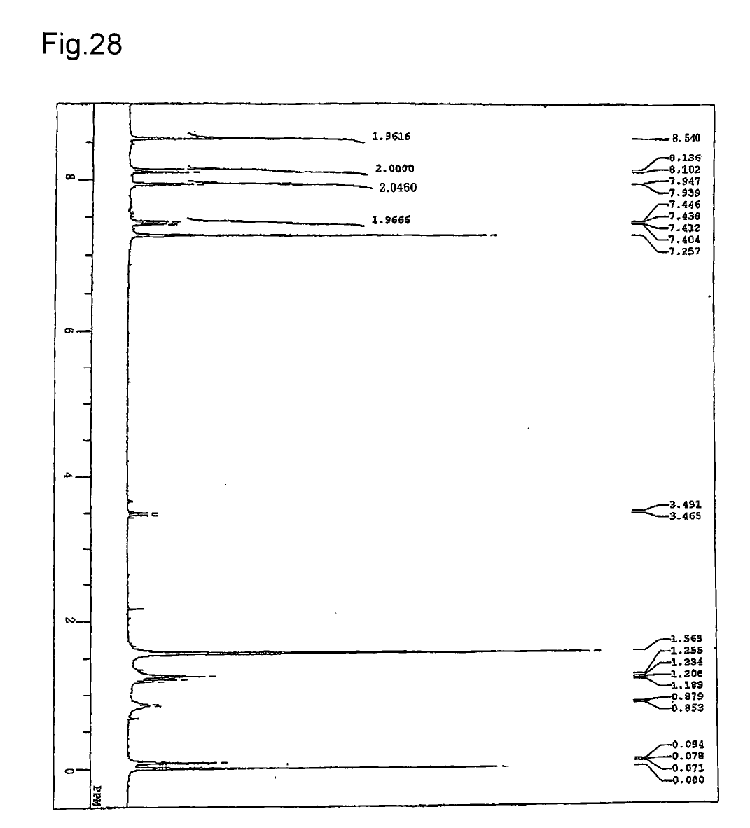

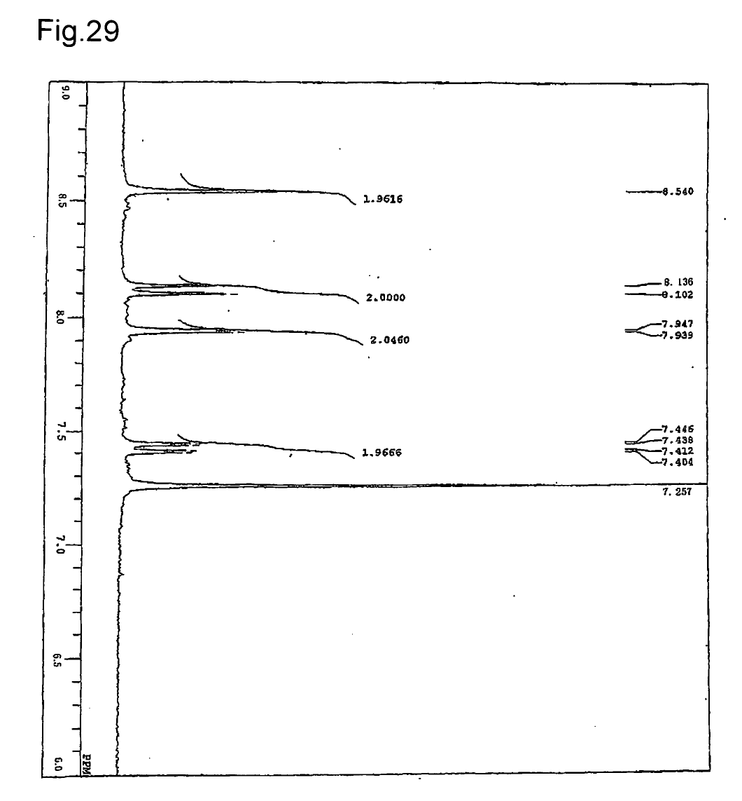

20 ml of a dichloromethane solution containing 128.0 mg (0.62 mmol) of the 2,6-dihydroxyanthracene obtained as described above was added with 0.15 ml (1.85 mmol) of pyridine. Then, 0.41 ml (2.46 mmol) of trifluoromethanesulfonic acid (Tf₂O) was added dropwise to the solution under a temperature condition of 0° C., and vigorously stirred. The reaction was traced with TLC. Even after more than 15 hours of stirring, there still remains the raw material. For this reason, pyridine (5 eq.) and Tf₂O (6 eq.) were further added three separate times. After the reaction was completed, the organic phase was extracted with dichloromethane. Subsequently, the organic phase was washed with saturated sodium acid carbonate and brine, dried with anhydrous sodium sulfate, and concentrated under a reduced pressure to obtain a reaction product. The reaction product thus obtained was purified by silica gel column chromatography (EtOAc), and thereby an anthracene compound expressed by the general formula (82) was obtained (a yield of 261.4 mg and 90%). The following reaction formula (G) shows an outline of the synthesis method for the anthracene compound. Moreover, the NMR measurement results of the anthracene compound are shown in FIGS. 28 and 29.

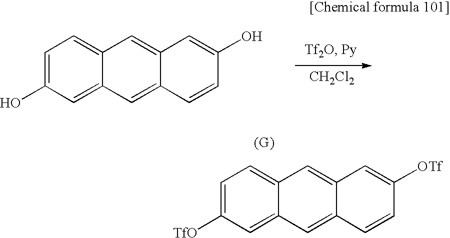

### Production of 2,6-bis(triethoxysilyl)anthracene

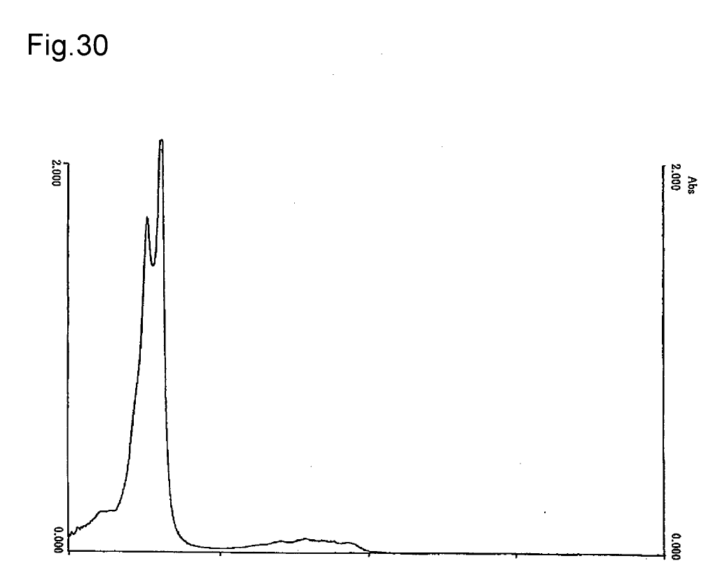

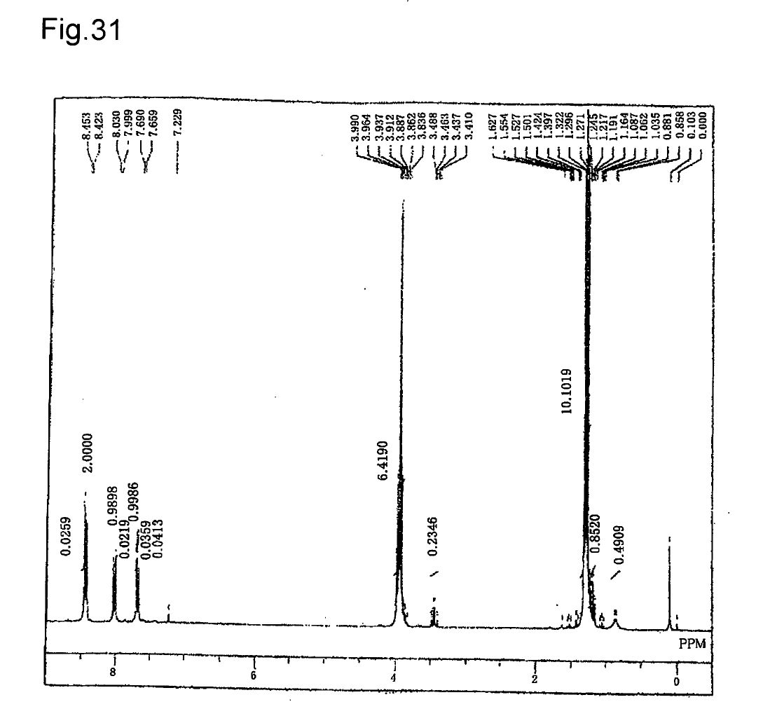

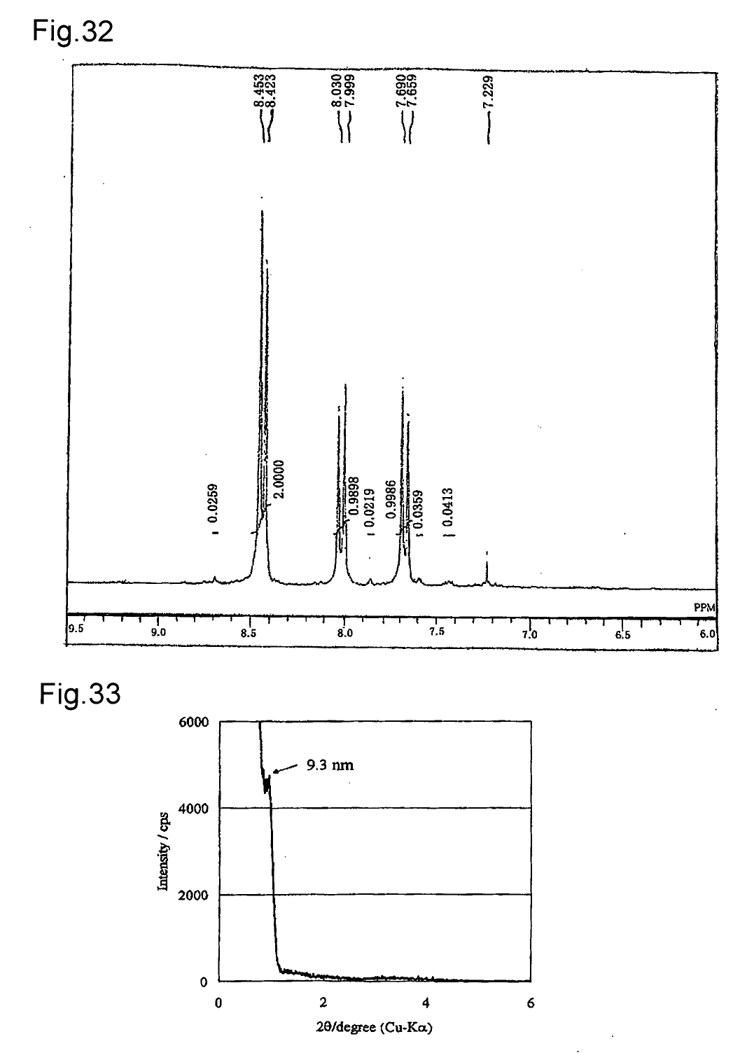

1.57 g (3.32 mmol) of the anthracene compound obtained as described above and expressed by the general formula (82) (2,6-dihydroxyanthracene), 75.6 mg (0.2 mmol) of [Rh(CH₃CN)₂(cod)]BF₄, and 2.45 g (6.64 mmol) of Bu₄NI were added into a reaction container, and dissolved in 43 ml of dimethylformamide (distilled DMF) to obtain a mixed solution. Then, the mixed solution was added with 2.78 ml (19.9 mmol) of triethanolamine (TEA), and added dropwise with 2.45 ml (13.3 mmol) of triethoxysilane under a temperature condition of 0° C. to obtain a suspension. Subsequently, the suspension thus obtained was stirred under a nitrogen atmosphere and a temperature condition of 80° C. for 2 hours. Thereafter the obtained suspension was concentrated, filtered with celite, and further concentrated to obtain an anthracene-silane compound (a yield of 1.65 g and 99%). The UV spectrum of the anthracene-silane compound thus obtained is shown in FIG. 30. The NMR measurement results are shown in FIGS. 31 and 32. The obtained anthracene-silane compound was 2,6-bis(triethoxysilyl)anthracene.

### Example 7

0.08 g of a triblock copolymer P123 ((EO)₂₀(PO)₇₀(EO)₂₀) was dissolved in a solution which had been prepared by adding 43 μl of ion-exchanged water and 10 μl of a 2N hydrochloric acid solution to 2 g of a mixed solvent of ethanol/THF (weight ratio of 1:1). Then, the resultant solution was added with 0.1 g of 2,7-BTEFlu having a structure expressed by the following general formula (86), and stirred at room temperature for 20 hours or longer. Thereby, a sol solution was obtained. Using this sol solution, a coating film (film thickness: 100 nm to 300 nm) was obtained by a spin coating method. Note that, in the coating conditions, the revolution speed was 4000 rpm, and the revolution time was 1 minute. Subsequently, the obtained film was dried at 100° C. for 1 hour or longer.

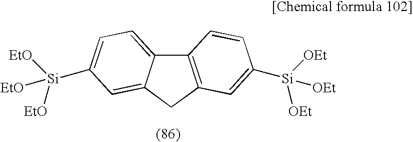

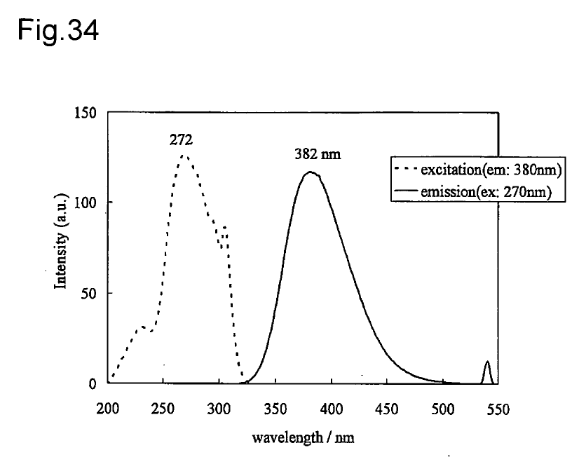

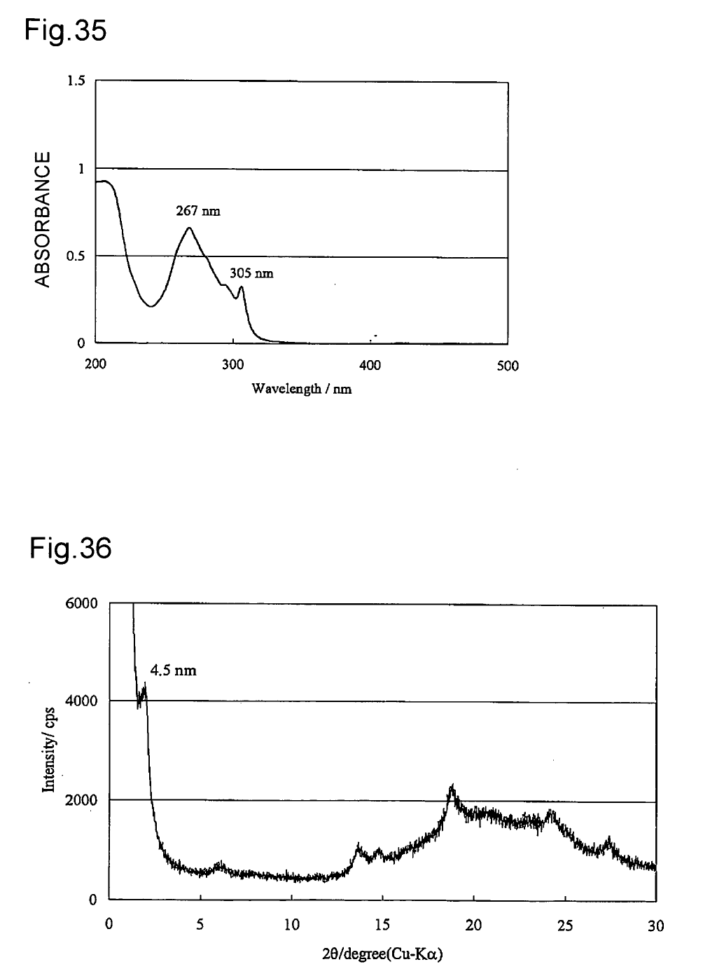

An X-ray diffraction pattern of the fluorene-silane compound thin film (Flu-HMM-s-film), a fluorescence spectrum and an excitation spectrum thereof, and a UV spectrum thereof are respectively shown in FIGS. 33, 34, and 35. In the X-ray diffraction pattern, the peak was observed at d=9.3 nm, and therefore it was confirmed that an ordered mesostructure was present (FIG. 33). Additionally, when the fluorescence spectrum was measured with an excitation wavelength of 270 nm, it was found that a strong emission was shown around 380 nm (FIG. 34). Moreover, based on the UV spectrum result, it was found that the light absorption bands were present around approximately 279 nm and 305 nm (FIG. 35).

### Example 8

0.154 g of 1,12-bis(octadecyldimethylammonium)dodecan dibromide (C₁₈₋₁₂₋₁₈) was dissolved in a solution in which 667 μl of 12N hydrochloric acid aqueous solution had been added to 12 g of ion-exchanged water. Then, the solution was added with 0.2 g of 2,7-BTEFlu, and vigorously stirred. The resultant solution was subjected to an ultrasonic treatment for 2 minutes, and stirred at room temperature for 24 hours. Subsequently, the solution was further stirred at 40° C. for 3 days, filtered, and dried. Thereby, a mesostructured powder made of a fluorene-disilane-compound was obtained.

An X-ray diffraction pattern of the powder (Flu-HMM-powder) thus obtained and a fluorescence spectrum and an excitation spectrum thereof are respectively shown in FIGS. 36, and 37. In the X-ray diffraction pattern, a peak based on the mesostructure was observed at d=4.5 nm, and therefore it was confirmed that an ordered mesostructure was present (FIG. 36). Additionally, when the fluorescence spectrum was measured with an excitation wavelength of 320 nm, it was found that a strong emission was shown around 385 nm (FIG. 37).

### Example 9

A solution in which 1 g of a mixed solvent of ethanol/THF (weight ratio of 1:1) had been added with 21 μl of ion-exchanged water, 5 μl of a 2N hydrochloric acid aqueous solution, and 0.07 g of Brij-76 (C₁₈H₃₇(EO)₁₀) was added with a solution in which 0.1 g of 1,6-BTEPyr having a structure expressed by the following general formula (91) had been dissolved in 1 g of a mixed solvent of ethanol/THF (weight ratio of 1:1). Then, the resultant solution was stirred at room temperature for 15 hours. Thereby, a sol solution was obtained. Using this sol solution, a coating film (film thickness: 100 nm to 300 nm) was obtained by a spin coating method. Subsequently, the film thus obtained was dried. In the coating conditions, the revolution speed was 4000 rpm, and the revolution time was 1 minute. The obtained film was dried at 100° C. for 1 hour or longer.

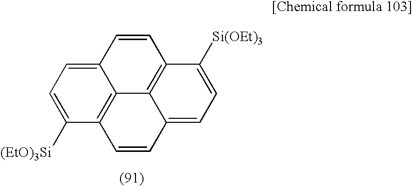

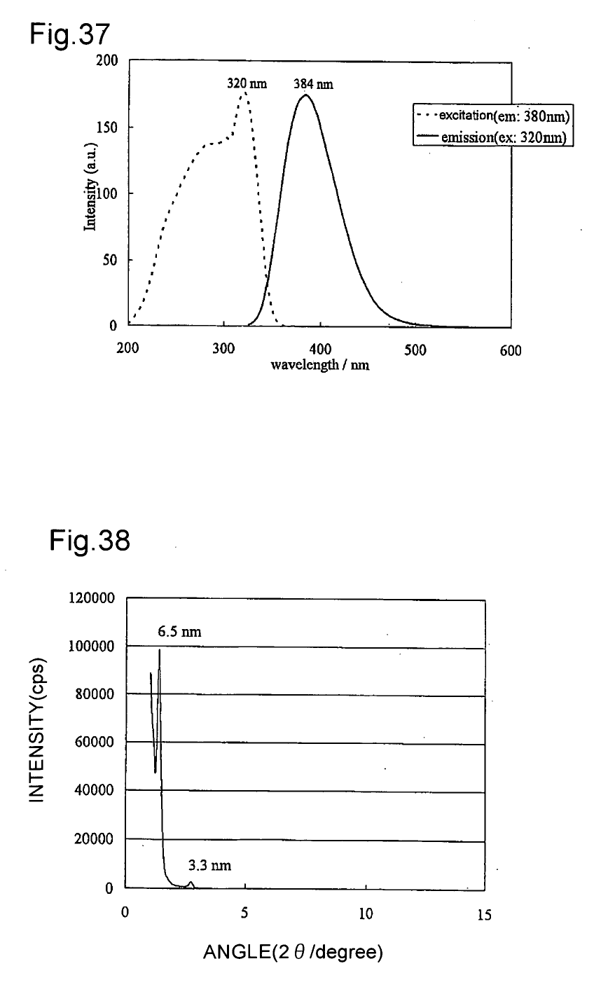

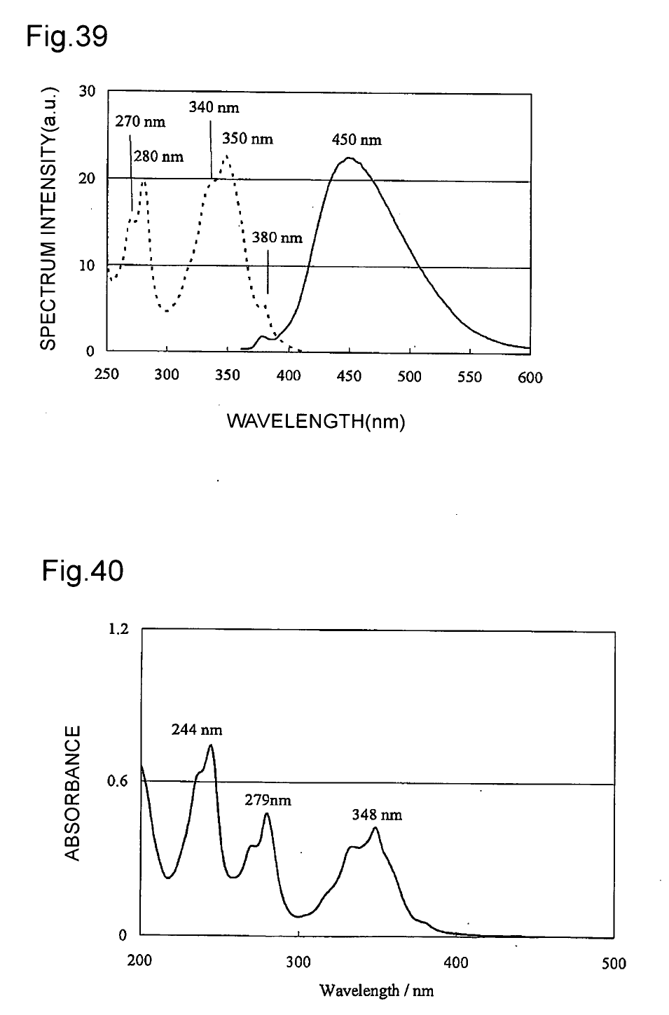

An X-ray diffraction pattern of the pyrene-silane-compound thin film (Pyr-HMMc-s-film) obtained in Example 9, a fluorescence spectrum (solid line, excitation wavelength: 350 nm) and excitation spectrum (dashed line, measured wavelength: 450 nm) thereof, and a UV spectrum thereof are respectively shown in FIGS. 38, 39, and 40. In the X-ray diffraction pattern, the strong peak was observed at d=6.5 nm, and therefore it was confirmed that an ordered mesostructure was present (FIG. 38). Additionally, when a fluorescence spectrum was measured with an excitation wavelength of 350 nm, it was found that a strong emission was shown around 450 nm (FIG. 39). Moreover, based on the UV spectrum result, it was found that the light absorption bands were present around approximately 245 nm, 280 nm, and 350 nm (FIG. 40).

### Example 10

A solution in which 1 g of ethanol had been added with 10 μl of ion-exchanged water, and 2 μl of a 2N hydrochloric acid aqueous solution was added with a solution in which 0.1 g of 1,6-BTEPyr had been dissolved in 1 g of ethanol. Then, the resultant solution was stirred at room temperature for 1 hour. Thereby, a sol solution was obtained. Using this sol solution, a coating film (film thickness: 100 nm to 300 nm) was obtained by a spin coating method as in Example 22. Subsequently, the film thus obtained was dried.

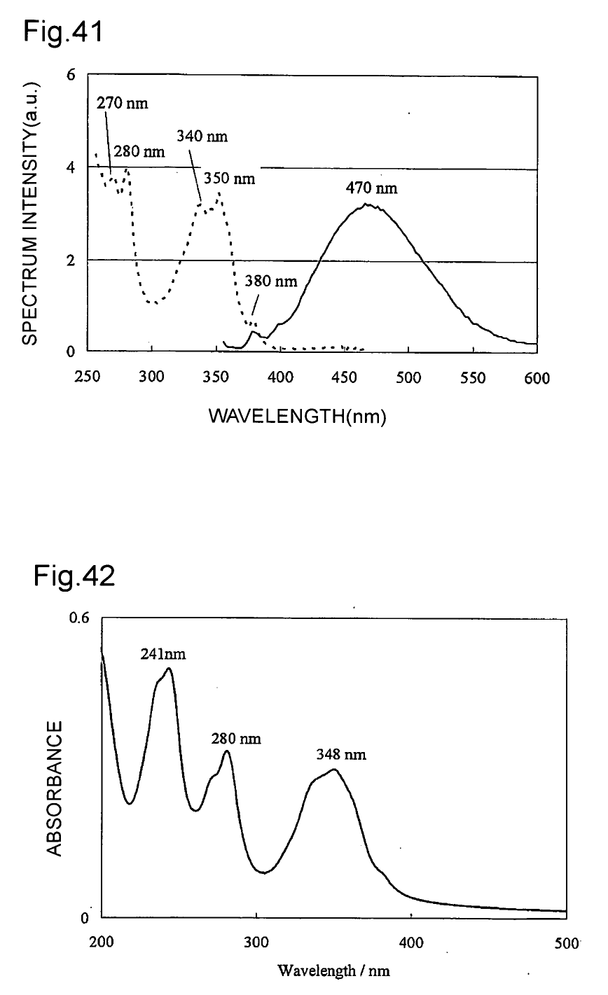

A fluorescence spectrum (solid line, excitation wavelength: 350 nm) and excitation spectrum (dashed line, measured wavelength: 450 nm) of the pyrene-silane-compound thin film (Pyr-acid-film) obtained in Example 10, and a UV spectrum thereof are respectively shown in FIGS. 41 and 42. When a fluorescence spectrum was measured with an excitation wavelength of 350 nm, it was found that a strong emission was shown around 470 nm (FIG. 41). Moreover, based on the UV spectrum result, it was found that the light absorption bands were present around approximately 240 nm, 280 nm, and 350 nm (FIG. 42).

### Example 11

0.07 g of Brij-76 (C₁₈H₃₇(EO)₁₀) as a nonionic surfactant was dissolved in a solution in which 1 g of a mixed solvent of ethanol/THF (weight ratio of 1:1) had been added with 21 μl of ion-exchanged water and 5 μl of a 2N hydrochloric acid aqueous solution. This solution was added with a solution in which 0.1 g of 1,8-BTEPyr having a structure expressed by the following general formula (92) had been dissolved in 1 g of a mixed solvent of ethanol/THF (weight ratio of 1:1). Then, the resultant solution was stirred at room temperature for 15 hours. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at 100° C. for 1 hour or longer.

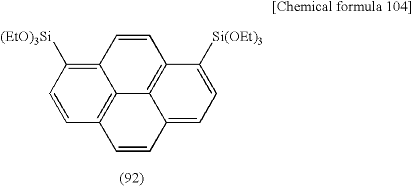

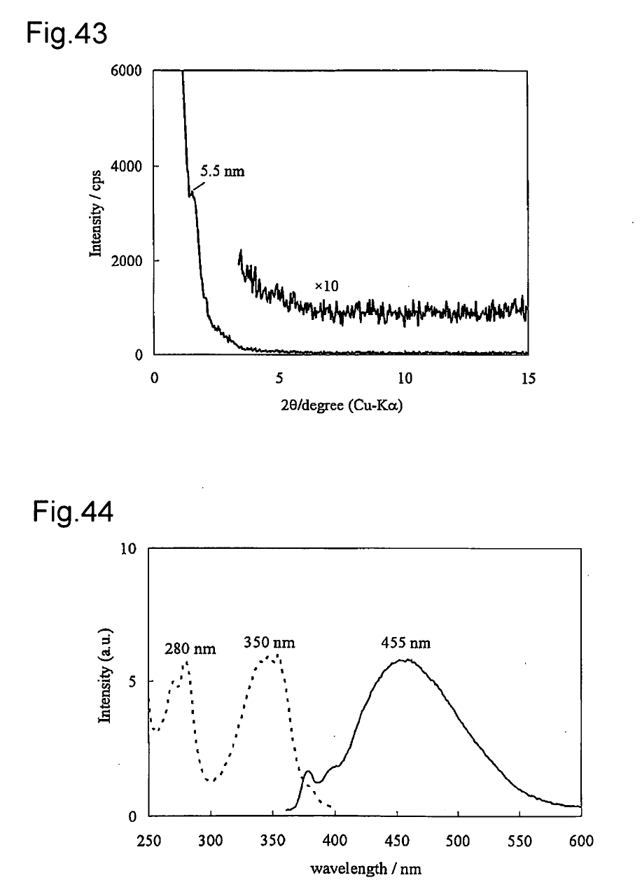

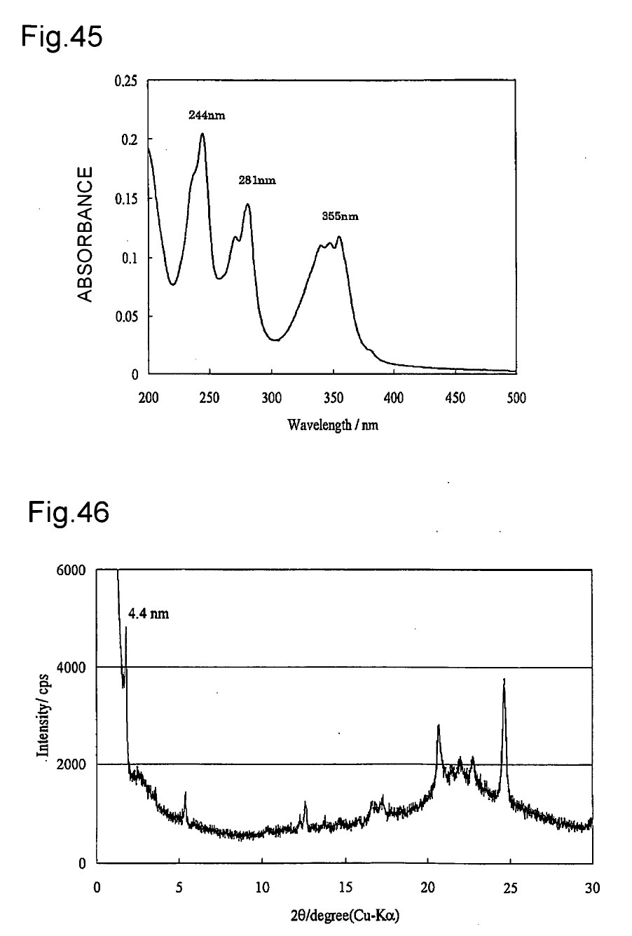

An X-ray diffraction pattern of the obtained pyrene-silane-compound thin film (Pyr-HMM-s-film), a fluorescence spectrum and an excitation spectrum thereof, and a UV spectrum thereof are respectively shown in FIGS. 43, 44, and 45. In the X-ray diffraction pattern, the strong peak was observed at d=6.5 nm, and therefore it was confirmed that an ordered mesostructure was present (FIG. 43). When a fluorescence spectrum was measured with an excitation wavelength of 350 nm, it was found that a strong emission having a peak at 450 nm was shown (FIG. 44). Moreover, based on the UV spectrum result, it was found that the light absorption bands were present around approximately 245 nm, 280 nm and 350 nm (FIG. 45).

### Example 12

0.08 g of 1,12-bis(octadecyldimethylammonium)dodecan dibromide (C₁₈₋₁₂₋₁₈) was dissolved in a solution in which 333 μl of a 12N hydrochloric acid aqueous solution had been added to 6 g of ion-exchanged water. Then, the solution was added with a solution in which 0.1 g of 1,6-BTEPyr had been dissolved in 1 g of ethanol (EtOH), and was vigorously stirred. The resultant solution was subjected to an ultrasonic treatment for 15 minutes, and then stirred at room temperature for 24 hours. Subsequently, the solution was heated at 100° C. for 20 hours, filtered, and dried. Thereby, a mesostructured powder made of a pyrene-silane-compound was obtained.

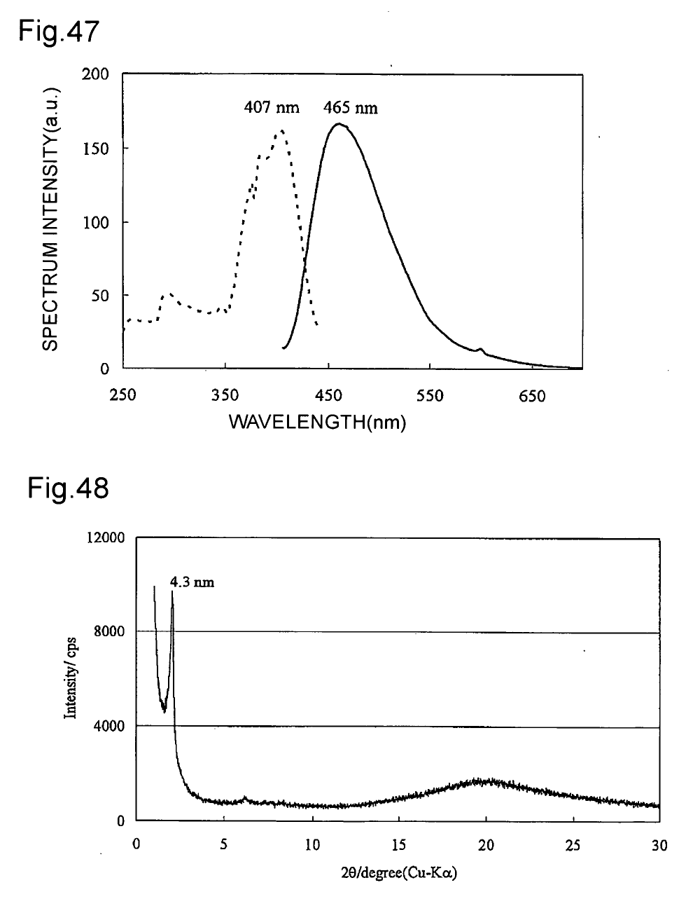

An X-ray diffraction pattern of the obtained powder (Pyr-Acid-powder) and a fluorescence spectrum and an excitation spectrum thereof are respectively shown in FIGS. 46 and 47. In the X-ray diffraction pattern, a peak based on the mesostructure was observed at d=4.4 nm, and therefore it was confirmed that an ordered mesostructure was present (FIG. 46). Additionally, when a fluorescence spectrum was measured with an excitation wavelength of 400 nm, it was found that a strong emission was shown around 465 nm (FIG. 47).

### Example 13

0.08 g of 1,12-bis(octadecyldimethylammonium)dodecan dibromide (C₁₈₋₁₂₋₁₈) was dissolved in a solution in which 333 μl of a 12N hydrochloric acid aqueous solution had been added to 6 g of ion-exchanged water. Then, the solution was added with a solution in which 0.1 g of 2,6-BTEAnt having a structure expressed by the following general formula (93) had been dissolved in 1 g of ethanol, and was vigorously stirred. After being subjected to an ultrasonic treatment for 15 minutes, the solution was stirred at room temperature for 24 hours. Thereafter, the solution was heated at 100° C. for 20 hours, filtered, and dried. Thereby, a mesostructured powder made of a anthracene-silane-compound was obtained.

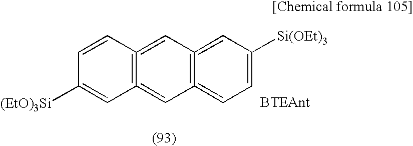

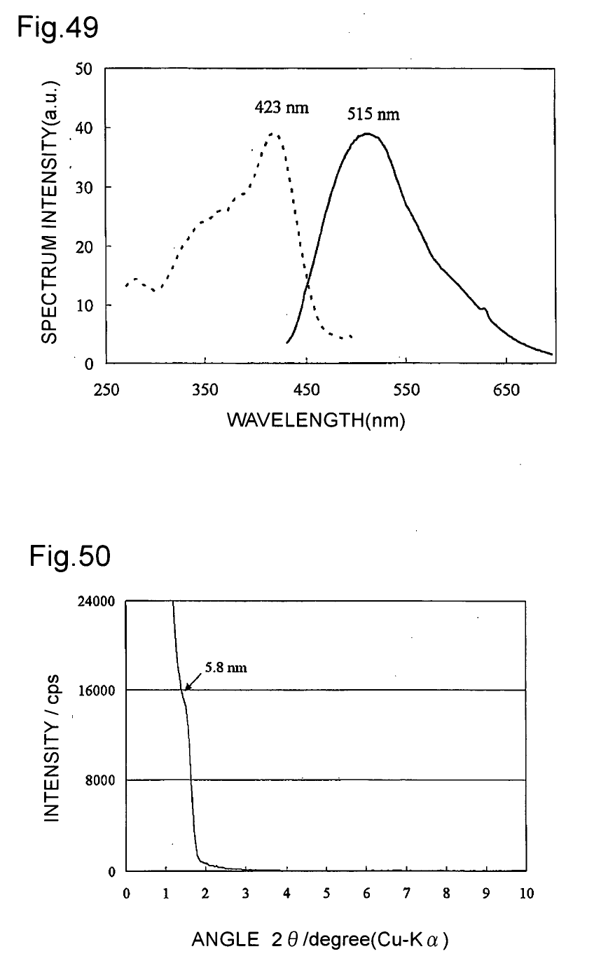

An X-ray diffraction pattern of the obtained powder (Ant-Acid-powder) and a fluorescence spectrum and an excitation spectrum thereof are respectively shown in FIGS. 48 and 49. In the X-ray diffraction pattern, a peak based on the mesostructure was observed at d=4.3 nm, and therefore it was confirmed that an ordered mesostructure was present (FIG. 48). Additionally, when a fluorescence spectrum was measured with an excitation wavelength of 420 nm, it was found that a strong emission was shown around 515 nm (FIG. 49).

### Example 14

0.07 g of Brij-76 (C₁₈H₃₇(EO)₁₀) as a nonionic surfactant was dissolved in a solution in which 43 μl of ion-exchanged water and 10 μl of 2N HCl had been added to 1 g of a mixed solvent of ethanol/THF (weight ratio of 1:1). Then, the solution was added with a solution in which 0.1 g of BTEAnt had been dissolved in 1 g of a mixed solvent of ethanol/THF (weight ratio of 1:1), and stirred at room temperature for 20 hours or longer. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at 100° C. for 1 hour or longer.

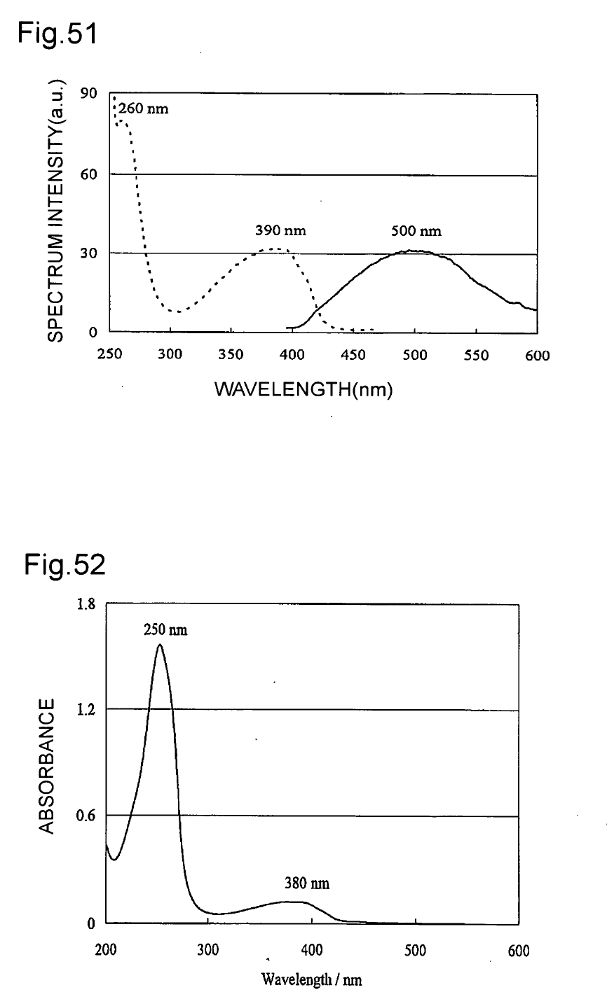

An X-ray diffraction pattern of the obtained anthracene-silane-compound thin film (Ant-HMM-s-film), a fluorescence spectrum and an excitation spectrum thereof, and a UV spectrum thereof are respectively shown in FIGS. 50, 51, and 52. In the X-ray diffraction pattern, although being broad, a peak was observed at d=5.8 nm, and therefore it was confirmed that an ordered mesostructure was present (FIG. 50). Additionally, when a fluorescence spectrum was measured with an excitation wavelength of 390 nm, it was found that a strong emission was shown around 500 nm (FIG. 51). Moreover, from the UV spectrum result, it was found that the light absorption bands were present around approximately 250 nm and 380 nm (FIG. 52).

### Example 15

0.08 g of a triblock copolymer P123 was dissolved in a solution in which 43 μl of ion-exchanged water and 10 μl of a 2N hydrochloric acid solution had been added to 2 g of a mixed solvent of ethanol/THF (weight ratio of 1:1). Then, the solution was added with 0.1 g of BTEAcr having a structure expressed by the following general formula (88), and stirred at room temperature for 20 hours or longer. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at 100° C. for 1 hour or longer.

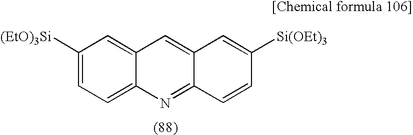

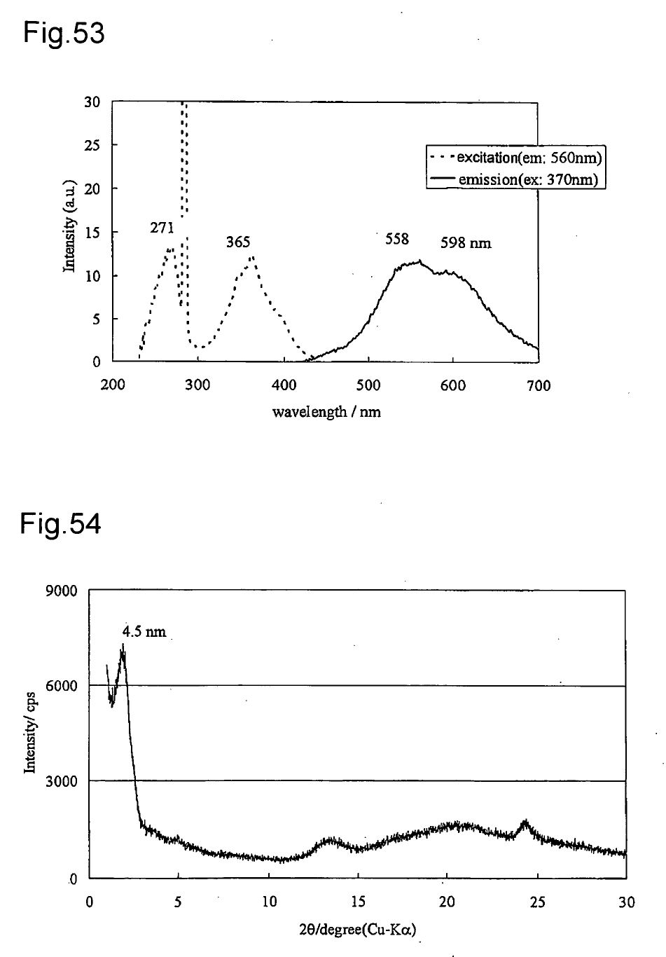

A fluorescence spectrum and an excitation spectrum of the acridine-silane-compound thin film (Acr-HMM-s-film) are shown in FIG. 53. When a fluorescence spectrum was measured with an excitation wavelength of 370 nm, it was found that an emission with a long wavelength was shown around 560 nm and 600 nm (FIG. 53). Meanwhile, in the X-ray diffraction pattern, a peak indicating a mesostructure was not recognized. It was assumed that the peak was concealed by the direct beam because the regularity of the mesostructure was not so high.

### Example 16

0.16 g of Octadecyltrimethylammonium chloride was dissolved in a solution in which 0.2 g of a 6 N NaOH aqueous solution had been added to 12 g of ion-exchanged water. Then, the solution was added with 0.2 g of 2,7-BTEAcr, and vigorously stirred. The resultant solution was subjected to an ultrasonic treatment for 15 minutes, and stirred at room temperature for 24 hours. Subsequently, the solution was heated at 100° C. for 20 hours, filtered, and dried. Thereby, a mesostructured powder made of an acridine-disilane-compound was obtained.

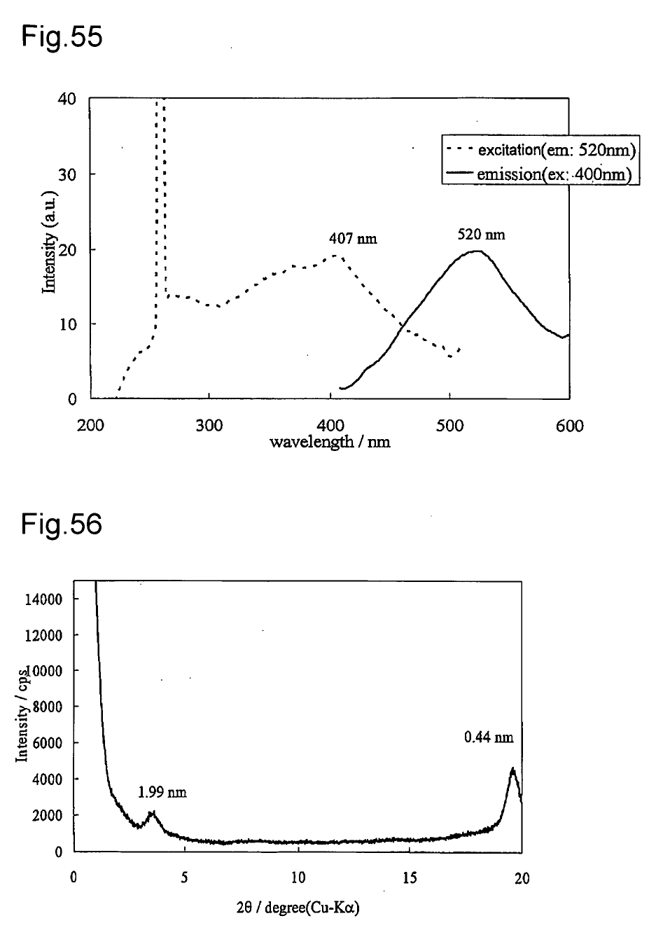

An X-ray diffraction pattern of the obtained powder (Acr-HMM-powder) and a fluorescence spectrum and an excitation spectrum thereof are respectively shown in FIGS. 54 and 55. In the X-ray diffraction pattern, a peak based on the mesostructure was observed at d=4.5 nm, and therefore it was confirmed that an ordered mesostructure was present (FIG. 54). Additionally, when a fluorescence spectrum was measured with an excitation wavelength of 400 nm, it was found that a strong emission was shown around 515 nm (FIG. 55).

### Example 17

0.08 g of 1,12-bis(octadecyldimethylammonium)dodecan dibromide (C₁₈₋₁₂₋₁₈) was dissolved in a solution in which 333 μl of a 12N hydrochloric acid aqueous solution had been added to 6 g of ion-exchanged water. Then, the solution was added with a solution in which 0.1 g of 4,4′″-bis(triethoxysilyl)quaterphenyl (4,4′″-BTEQua) had been dissolved in a mixed solvent of 1 g of ethanol and 0.5 g of THF, and vigorously stirred. The resultant solution was subjected to an ultrasonic treatment for 15 minutes, and stirred at room temperature for 24 hours. Subsequently, the solution was heated at 100° C. for 20 hours, filtered, and dried. Thereby, a quaterphenyl-silane-compound powder was obtained.

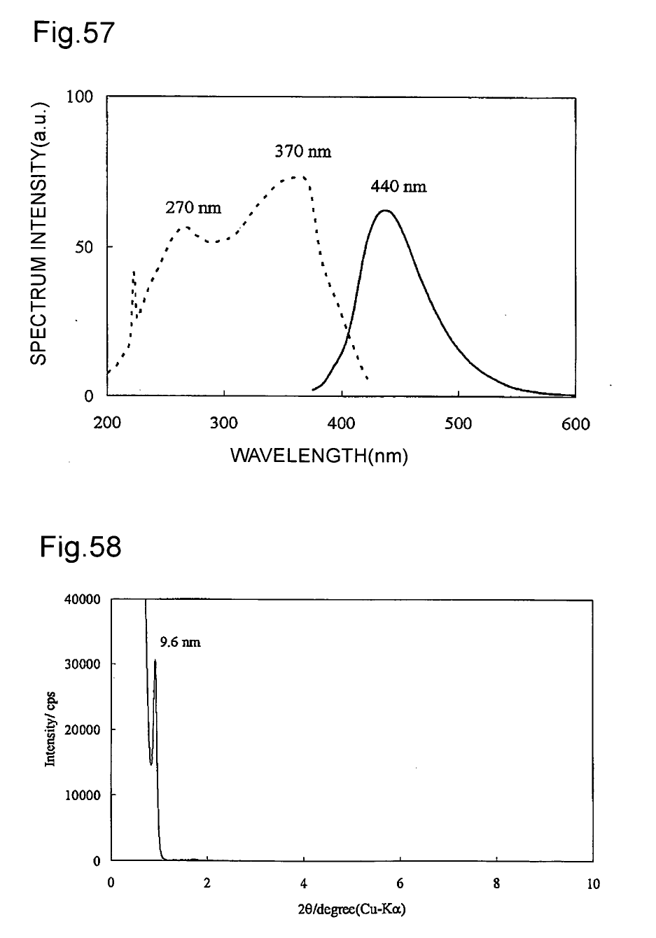

An X-ray diffraction pattern of the obtained quaterphenyl-silane-compound powder (Qua-HMM-powder) and a fluorescence spectrum and an excitation spectrum thereof are respectively shown in FIGS. 56 and 57. In the X-ray diffraction pattern, a peak indicating a mesostructure was not observed, but a peak based on the periodic structure of the quaterphenyl was observed at d=1.99 nm (FIG. 56). Additionally, when a fluorescence spectrum was measured with an excitation wavelength of 400 nm, it was found that a strong emission was shown around 465 nm (FIG. 57).

### Example 18

0.08 g of a triblock copolymer P123 was dissolved in a solution in which 43 μl of ion-exchanged water and 10 μl of a 2N hydrochloric acid aqueous solution had been added to 1 g of a mixed solvent of ethanol/THF (weight ratio of 1:1). Then, 0.1 g of BTEAcd having a structure expressed by the following general formula (89) was added to 1.5 g of a mixed solvent of ethanol/THF (weight ratio of 1:1), and stirred at room temperature for 1 hour. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at 100° C. for 1 hour or longer.

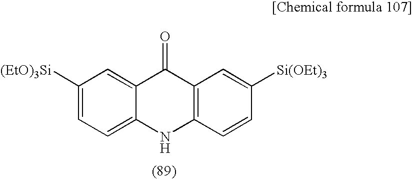

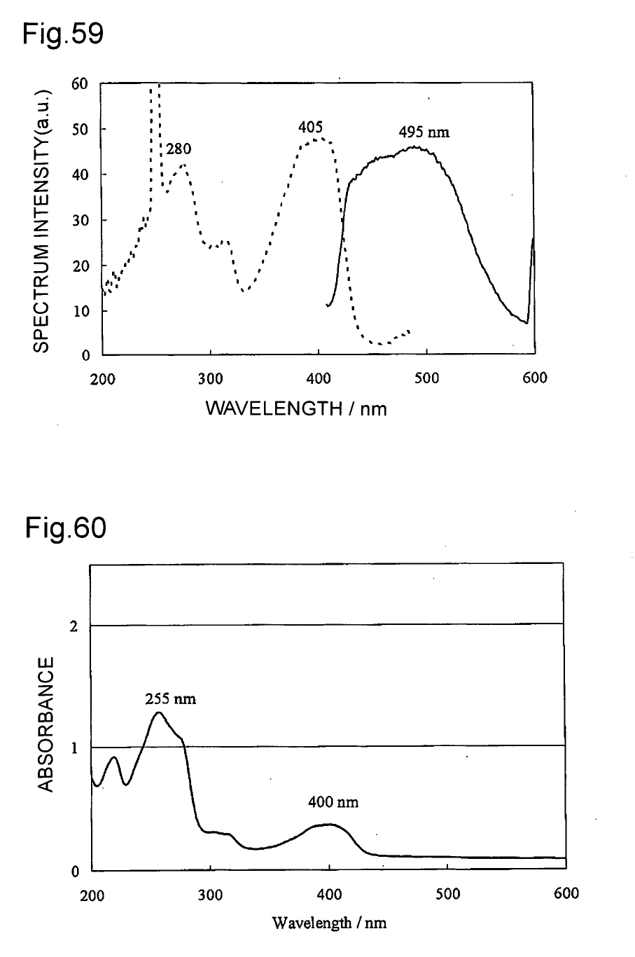

An X-ray diffraction pattern of the acridone-silane-compound thin film (Acd-HMM-s-film), a fluorescence spectrum and an excitation spectrum thereof, and a UV spectrum thereof are respectively shown in FIGS. 58, 59, and 60. In the X-ray diffraction pattern, a sharp peak was observed at d=9.6 nm, and therefore it was confirmed that an ordered mesostructure was present (FIG. 58). Additionally, when a fluorescence spectrum was measured with an excitation wavelength of 400 nm, it was found that a strong emission was shown around 500 nm (FIG. 59). Moreover, from the UV spectrum result, it was found that the light absorption bands were present around approximately 255 nm and 400 nm (FIG. 60).

### Example 19

0.16 g of octadecyltrimethylammonium chloride was dissolved in a solution in which 0.2 g of a 6 N NaOH aqueous solution had been added to 12 g of ion-exchanged water. Then, the solution was added with a solution in which 0.2 g of BTEAcd had been dissolved in 1 g of ethanol, and vigorously stirred. The resultant solution was subjected to an ultrasonic treatment for 15 minutes, and stirred at room temperature for 24 hours. Subsequently, the solution was heated at 100° C. for 24 hours, filtered, and dried. Thereby, a mesostructured powder made of an acridine-silane-compound was obtained.

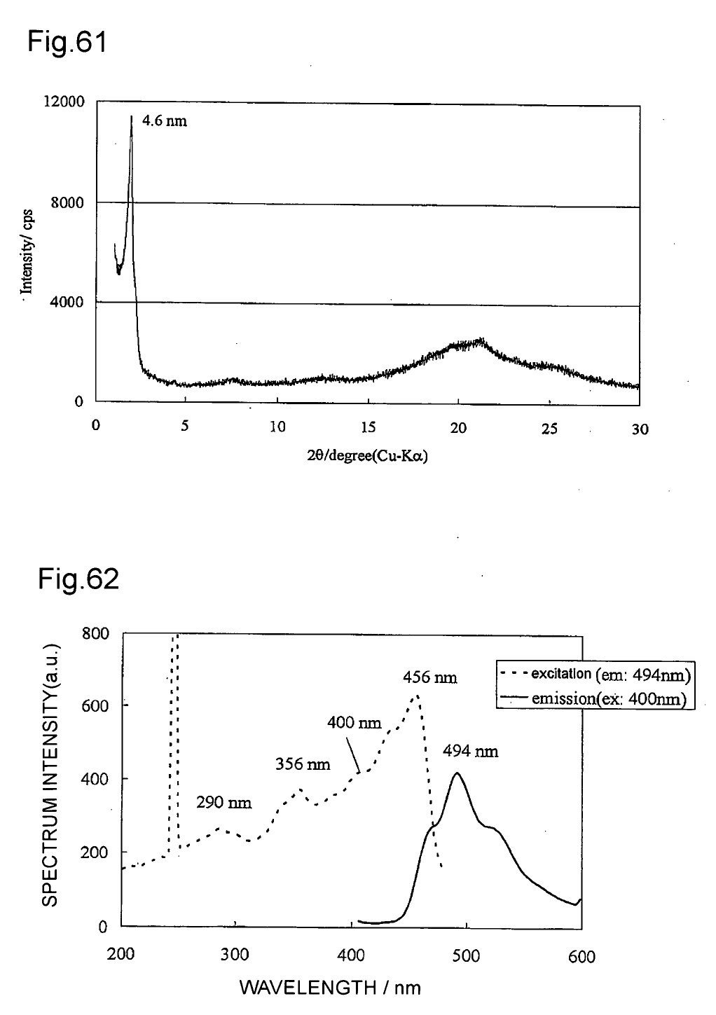

An X-ray diffraction pattern of the acridone-silica-composite-material powder thus obtained (Acd-HMM-powder), and a fluorescence spectrum and an excitation spectrum thereof are respectively shown in FIGS. 61 and 62. In the X-ray diffraction pattern, a peak based on the mesostructure was observed at d=4.6 nm, and therefore it was confirmed that an ordered mesostructure was present (FIG. 61). Additionally, when a fluorescence spectrum was measured with an excitation wavelength of 400 nm, it was found that a strong emission was shown around 494 nm (FIG. 62).

### Example 20

### synthesis of 3,6-Bis(triethoxysilyl)carbazole)

### Synthesis of 3,6-diiodocarbazole

A mixture of 278 mg (0.75 mmol, 2.5 eq.) of bis(pyridine)iodonium tetrafluoroborate (IPy₂BF₄) and 50 mg (0.30 mmol) of carbazole was added with 8 mL of dichloromethane under a nitrogen atmosphere, and further added dropwise with 26.4 μl (0.30 mmol, 1 eq.) of trifluoromethanesulfonic acid (TfOH) under a temperature condition of 0° C. Then, the resultant mixture was stirred under a nitrogen atmosphere at room temperature for 20 hours to obtain an orange-yellow reaction mixture (I). Subsequently, an excessive iodization reagent in the orange-yellow reaction mixture (I) thus obtained was decomposed with sodium thiosulfate (Na₂S₂O₃). Thereafter, the aqueous layer was extracted with dichloromethane. After that, the collected organic phase was washed with sodium chloride, dried with sodium sulfate (Na₂SO₄), filtered, and concentrated to obtain a crude product (I) (136.9 mg). Then, the crude product (I) thus obtained was separated and purified by silica gel chromatography (hexane:EtOAc=5:1). Thereby, 3,6-diiodocarbazole expressed by the following general formula (94) was obtained (a yield of 120.1 mg and 96%).

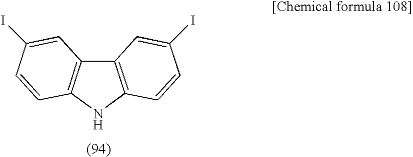

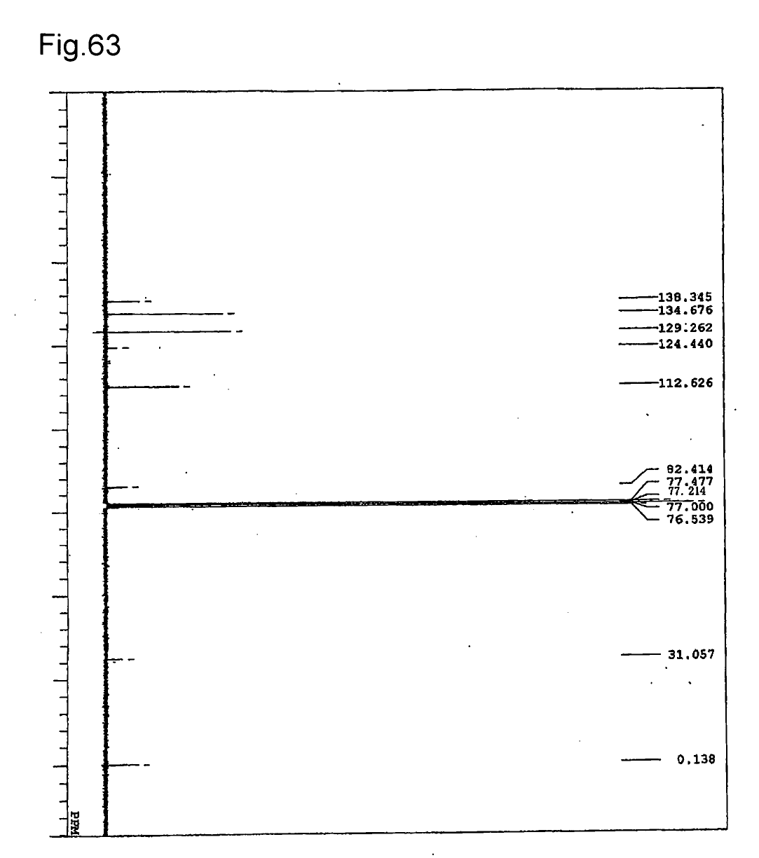

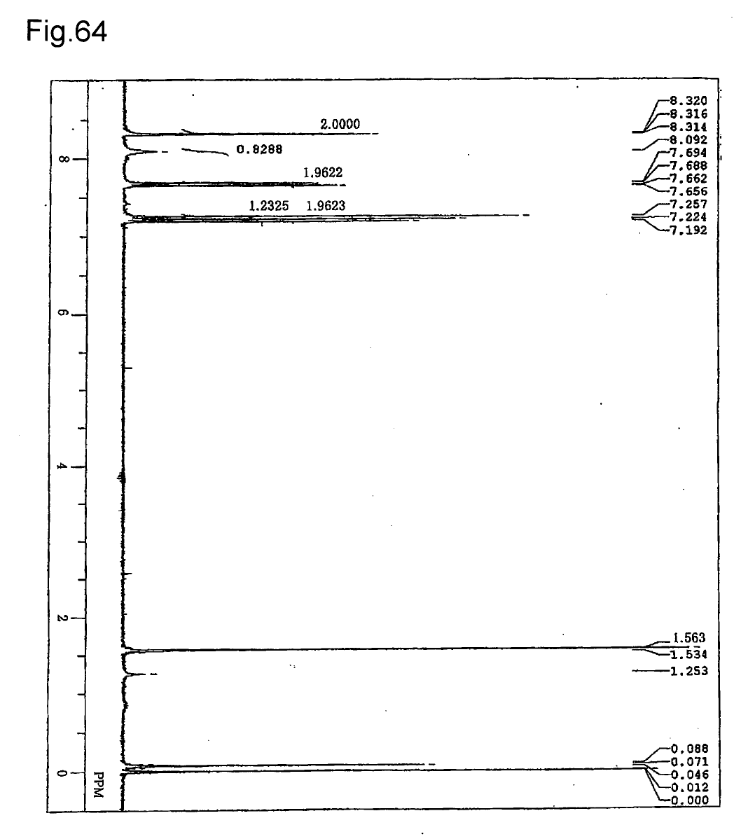

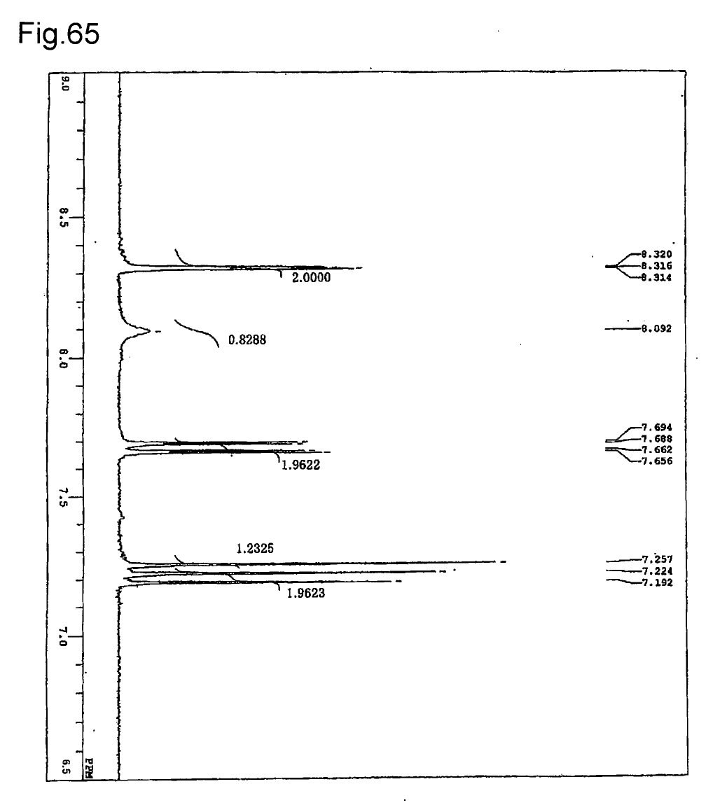

The 3,6-diiodocarbazole thus obtained was subjected to ¹³C NMR and ¹H NMR measurements. FIG. 63 shows a graph obtained from the ¹³C NMR measurement, and FIGS. 64 and 65 show graphs obtained from the ¹H-NMR measurements. These obtained results are shown below.

¹H NMR (CDCl₃) 8.32 (d, J=1.9 Hz, 2H), 8.09 (br, 1H), 7.68 (dd, J=8.4 Hz, 1.9 Hz, 2H), 7.22 (d, J=8.4 Hz, 2H);

¹³C NMR (CDCl₃) 138.34, 134.68, 129.26, 124.44, 112.63, 82.41.

### Synthesis of 3,6-Bis(triethoxysilyl)carbazole

A mixture of 1.0 g (2.39 mmol) of the 3,6-diiodocarbazole obtained as described above and 45 mg (0.12 mmol, 5 mol %) of [Rh(CH₃CN)₂(cod)]BF₄ was added with 20 mL of dimethylformamide (DMF) and 1.99 ml (27 mmol, 6 eq.) of triethylamine (TEA) under a nitrogen atmosphere. Then, the resultant mixture was stirred under a nitrogen atmosphere at room temperature for 30 minutes to obtain a mixed solution. Subsequently, the mixed solution thus obtained was added dropwise with 1.76 ml (18 mmol, 4 eq.) of triethoxysilane [(EtO)₃SiH] at room temperature, and stirred under a nitrogen atmosphere at 80° C. for 7 hours. Thereby, a reaction mixture (II) was obtained. Thereafter, the solvent in the reaction mixture (II) thus obtained was removed by distillation with a vacuum pump, and a residue was extracted with ether. After that, a salt thus formed was removed by filtering with celite. The solvent was removed by distillation from the organic phase with an evaporator to obtain a crude product (II). Then, the crude product (II) thus obtained was dissolved in 150 ml of ether, and purified by filtering the resultant through activated carbon (Kiriyama funnel, diameter: 5 cm, thickness: 7 mm). Thereby, a carbazole-silane compound was obtained (a yield of 1.097 g and 89%).

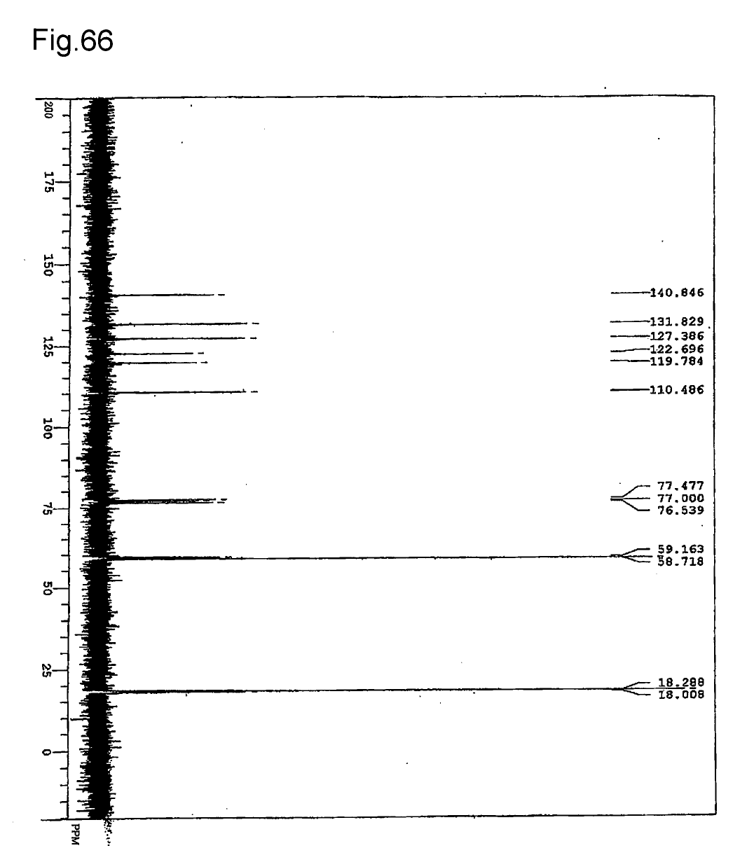

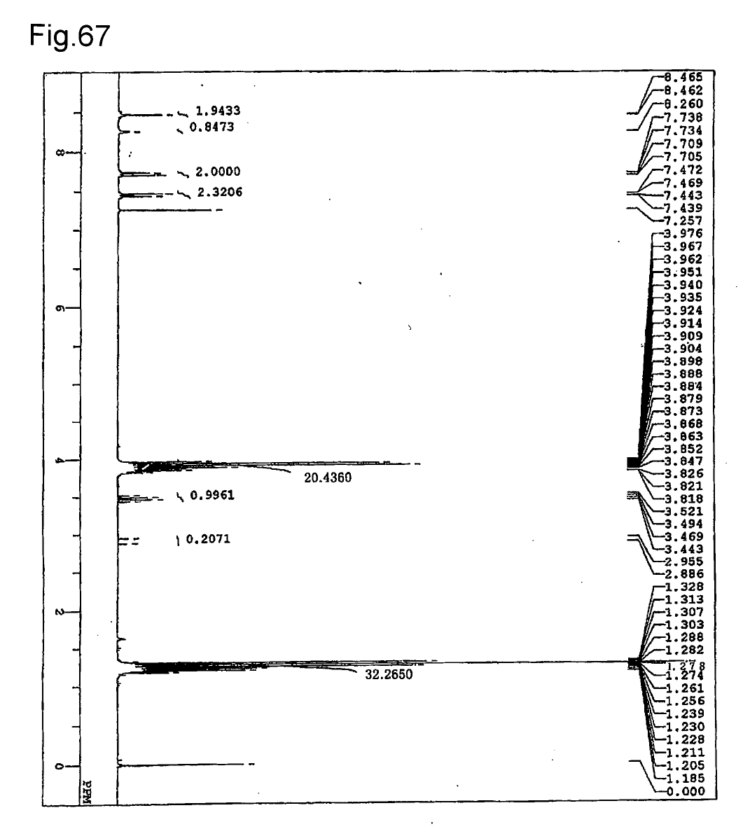

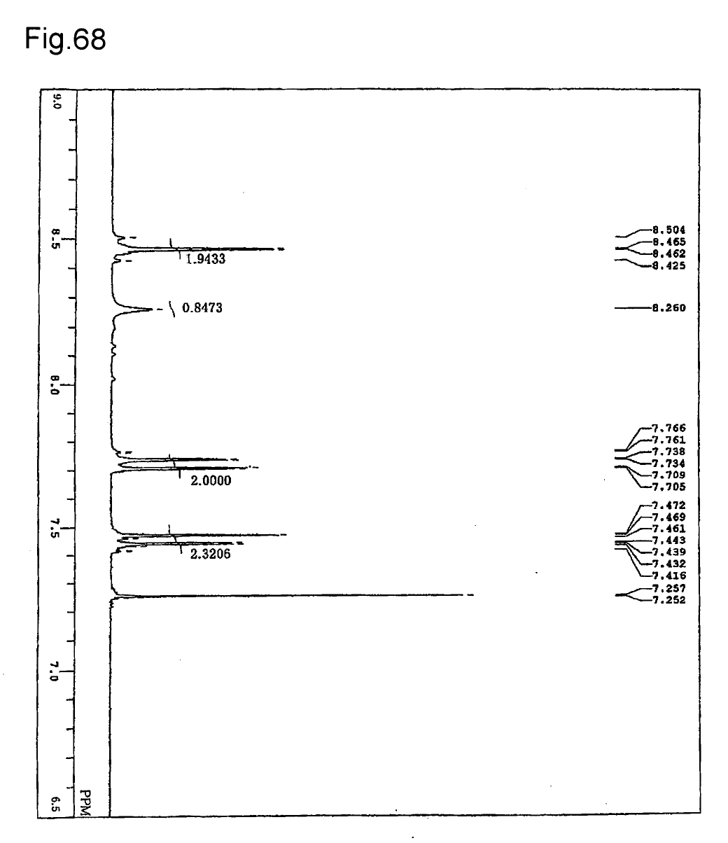

The carbazole-silane compound thus obtained was subjected to ¹³C NMR and ¹H NMR measurements. FIG. 66 shows a graph obtained from the ¹³C NMR measurement, and FIGS. 67 and 68 show graphs obtained from the ¹H-NMR measurements. These measurement results are shown below.

¹H NMR (CDCl₃) δ8.46 (d, J=0.8 Hz, 2H), 8.26 (s, 1H), 7.72 (dd, J=7.8 Hz, 0.8 Hz, 2H), 7.43 (dd, J=7.7, 0.8 Hz, 2H), 3.93 (q, J=7.3 Hz, 12H), 1.29 (t, J=7.3 Hz, 18H);

¹³C NMR (CDCl₃) δ140.85, 131.83, 127.39, 122.70, 119.78, 110.49, 58.72, 18.29.

Based on the NMR measurement results, it was confirmed that the carbazole-silane compound obtained in Example 20 was a carbazole-disilane compound expressed by the following general formula (95).

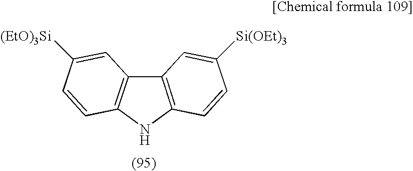

### Example 21

### synthesis of 3,6-Bis(triethoxysilyl)-9-methylcarbazole)

### Synthesis of 3,6-diiodo-9-methylcarbazole

A mixture of 308 mg (0.83 mmol, 2.5 eq.) of bis(pyridine)iodonium tetrafluoroborate (IPy₂BF₄) and 60 mg (0.33 mmol) of carbazole was added with 8 mL of dichloromethane under a nitrogen atmosphere, and further added dropwise with 29 μl (0.30 mmol, 1 eq.) of trifluoromethanesulfonic acid (TfOH) under a temperature condition of 0° C. Then, the resultant mixture was stirred under a nitrogen atmosphere at room temperature for 40 hours to obtain an orange-yellow reaction mixture (I). Subsequently, the excessive iodization reagent in the orange-yellow reaction mixture (I) thus obtained was decomposed with sodium thiosulfate (Na₂S₂O₃). Thereafter, the aqueous layer was extracted with dichloromethane. After that, the collected organic phase was washed with sodium chloride, dried with sodium sulfate (Na₂SO₄), filtered, and concentrated to obtain a crude product (I) (143.9 mg). Then, the crude product (I) thus obtained was separated and purified by silica gel chromatography (hexane:EtOAc=5:1). Thereby, 3,6-diiodo-9-methylcarbazole expressed by the following general formula (96) was obtained (a yield of 133.0 mg and 93%).

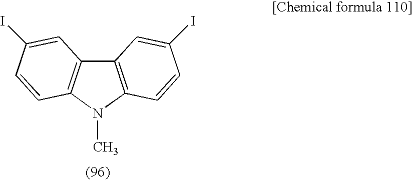

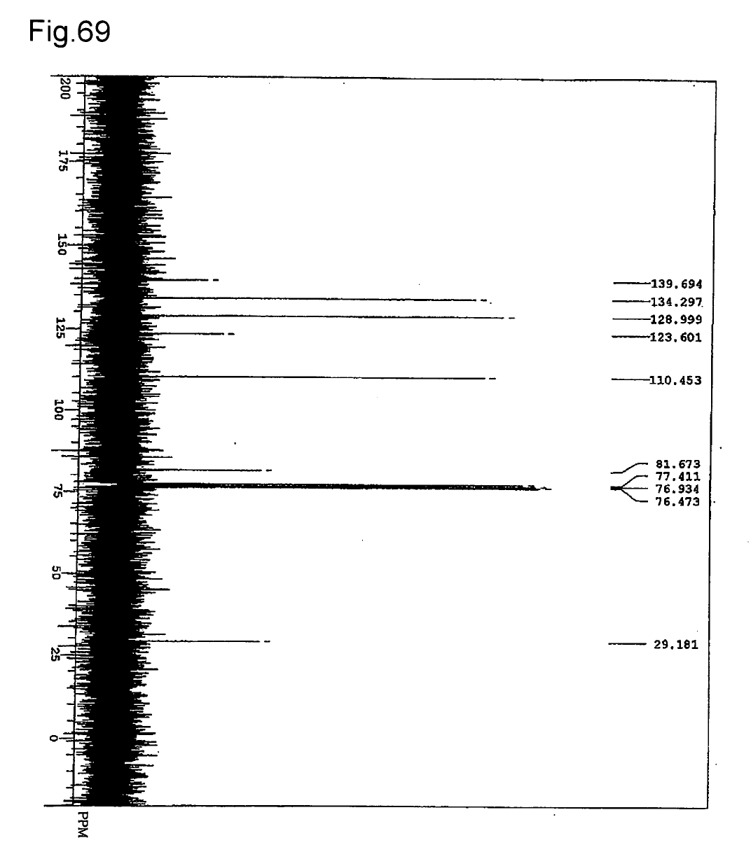

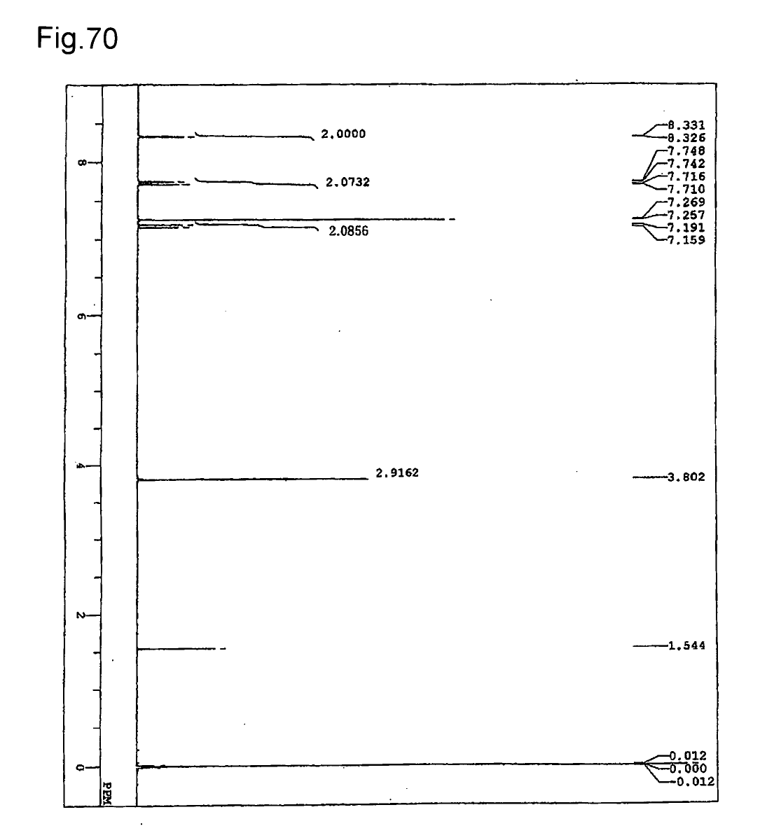

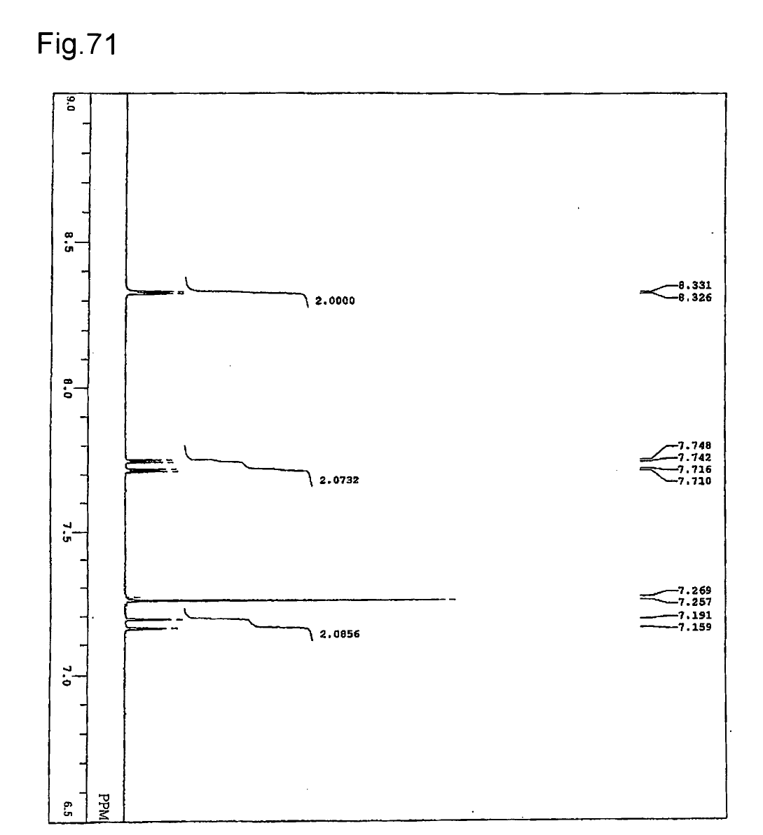

The obtained 3,6-diiodo-9-methylcarbazole was subjected to ¹³C NMR and ¹H NMR measurements. FIG. 69 shows a graph obtained from the ¹³C NMR measurement, and FIGS. 70 and 71 show graphs obtained from the ¹H-NMR measurements. These measurement results are shown below.

¹H NMR (CDCl₃) d8.32 (d, J=1.6 Hz, 2H), 7.73 (d, J=8.6 Hz, 1.6 Hz, 2H), 7.17 (d, J=8.6 Hz, 2H), 3.80 (s, 3H);

¹³C NMR (CDCl₃) d139.69, 134.30, 129.00, 123.60, 110.45, 81.67.

### Synthesis of 3,6-Bis(triethoxysilyl)-9-methylcarbazole

A mixture of 100 mg (0.23 mmol) of the 3,6-diiodo-9-methylcarbazole obtained as described above and 4.4 mg (0.012 mmol, 5 mol %) of [Rh(CH₃CN)₂(cod)]BF₄ was added with 4 ml of dimethylformamide (DMF) and 180 μl (1.39 mmol, 6 eq.) of triethylamine (TEA) under a nitrogen atmosphere. Then, the resultant mixture was stirred under a nitrogen atmosphere at room temperature for 30 minutes to obtain a mixed solution. Subsequently, the mixed solution thus obtained was added dropwise with 171 μl (0.92 mmol, 4 eq.) of triethoxysilane [(EtO)₃SiH] at room temperature, and stirred under a nitrogen atmosphere at 80° C. for 7 hours. Thereby, a reaction mixture (II) was obtained. Thereafter, the solvent in the reaction mixture (II) thus obtained was removed by distillation with a vacuum pump, and a residue was extracted with ether. After that, a salt thus formed was removed by filtering with celite. The solvent was removed by distillation from the organic phase with an evaporator to obtain a crude product (II). Then, the crude product (II) thus obtained was dissolved in 15 ml of ether, and purified by filtering the resultant through activated carbon (Kiriyama funnel, diameter: 1.5 cm, thickness: 5 mm). Thereby, a carbazole-silane compound was obtained (a yield of 90.9 g and 78%).

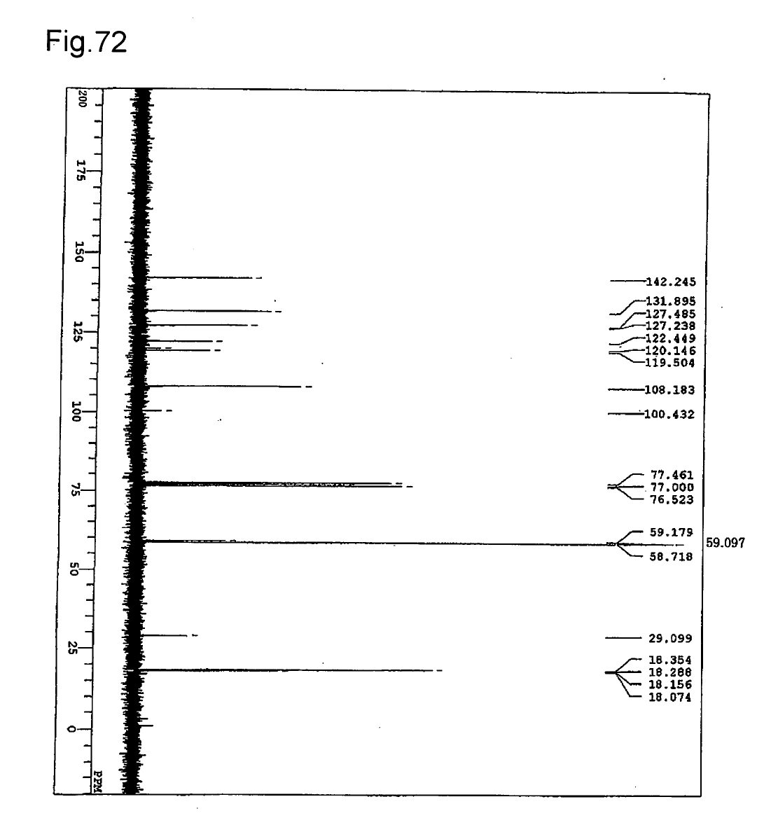

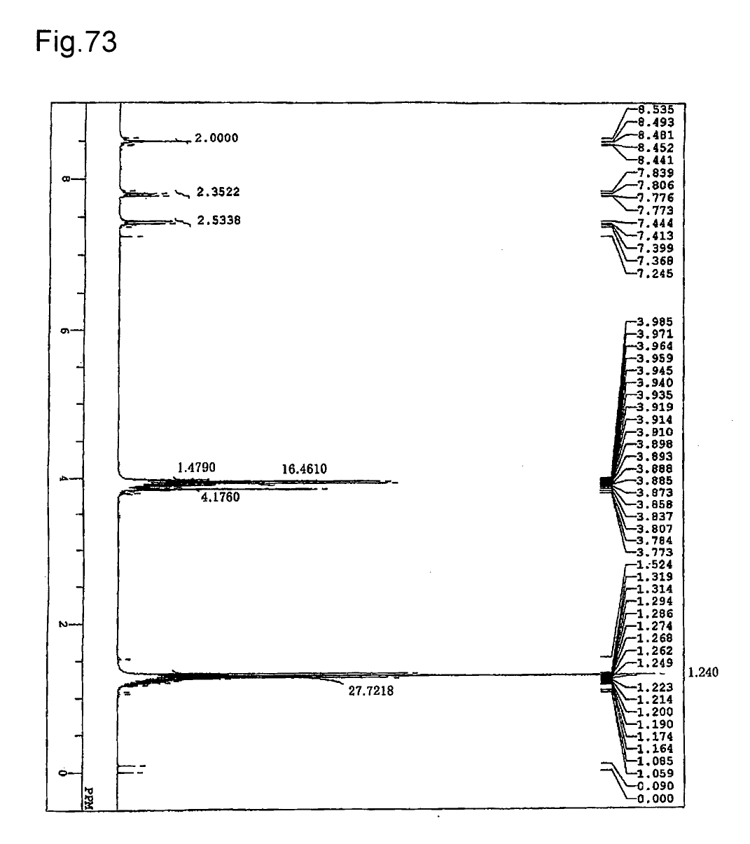

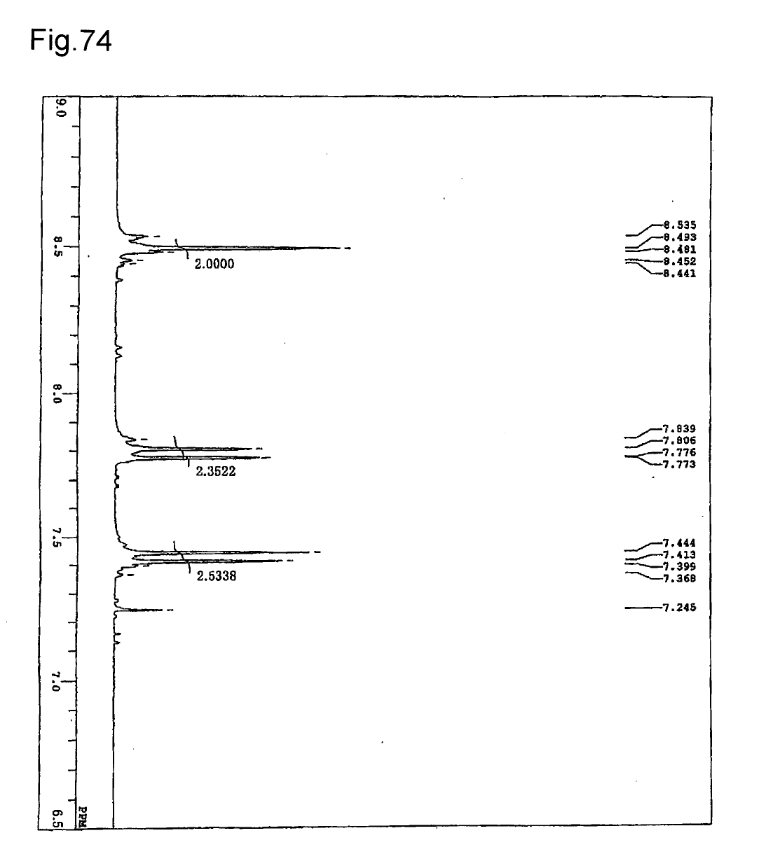

The obtained carbazole-silane compound was subjected to ¹³C NMR and ¹H NMR measurements. FIG. 72 shows a graph obtained from the ¹³C NMR measurement, and FIGS. 73 and 74 show graphs obtained from the ¹H-NMR measurements. These measurement results are shown below.

¹H NMR (CDCl₃) δ8.49 (s, 2H), 7.79 (d, J=8.1 Hz, 2H), 7.43 (d, J=8.1 Hz, 2H), 3.95 (q, J=7.1 Hz, 12H), 3.84 (s, 3H), 1.29 (t, J=7.1 Hz, 18H)

¹³C NMR (CDCl₃) δ142.25, 131.90, 127.49, 122.45, 119.50, 108.18, 58.72, 29.10, 18.35.

Based on the NMR measurement results, it was confirmed that the carbazole-silane compound obtained in Example 21 was a carbazole-disilane compound expressed by the following general formula (97).

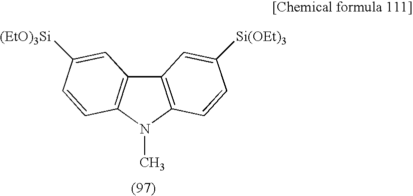

### Example 22

### synthesis of 3,6-Bis(triethoxysilyl)-9-oethylcarbazole)

### Synthesis of 3,6-diiodo-9-oethylcarbazole

A mixture of 166 mg (0.45 mmol, 2.5 eq.) of bis(pyridine)iodonium tetrafluoroborate (IPy₂BF₄) and 50 mg (0.18 mmol) of carbazole was added with 8 mL of dichloromethane under a nitrogen atmosphere, and further added dropwise with 32 μl (0.36 mmol, 2 eq.) of trifluoromethanesulfonic acid (TfOH) under a temperature condition of 0° C. Then, the resultant mixture was stirred under a nitrogen atmosphere at room temperature for 40 hours to obtain an orange-yellow reaction mixture (I). Subsequently, the excessive iodization reagent in the orange-yellow reaction mixture (I) thus obtained was decomposed with sodium thiosulfate (Na₂S₂O₃). Thereafter, the aqueous layer was extracted with dichloromethane. After that, the collected organic phase was washed with sodium chloride, dried with sodium sulfate (Na₂SO₄), filtered, and concentrated to obtain a crude product (I) (105 mg). Then, the crude product (I) thus obtained was separated by silica gel chromatography (hexane:EtOAc=20:1) and purified. Thereby, 3,6-diiodo-9-oethylcarbazole expressed by the following general formula (98) was obtained (a yield of 90 mg and 95%).

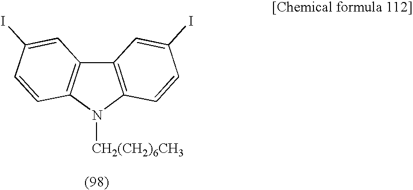

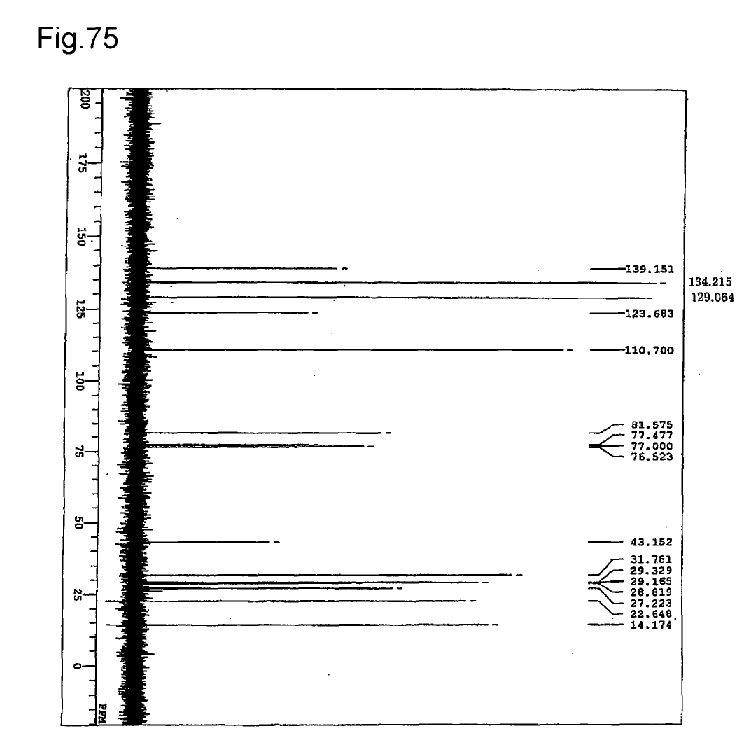

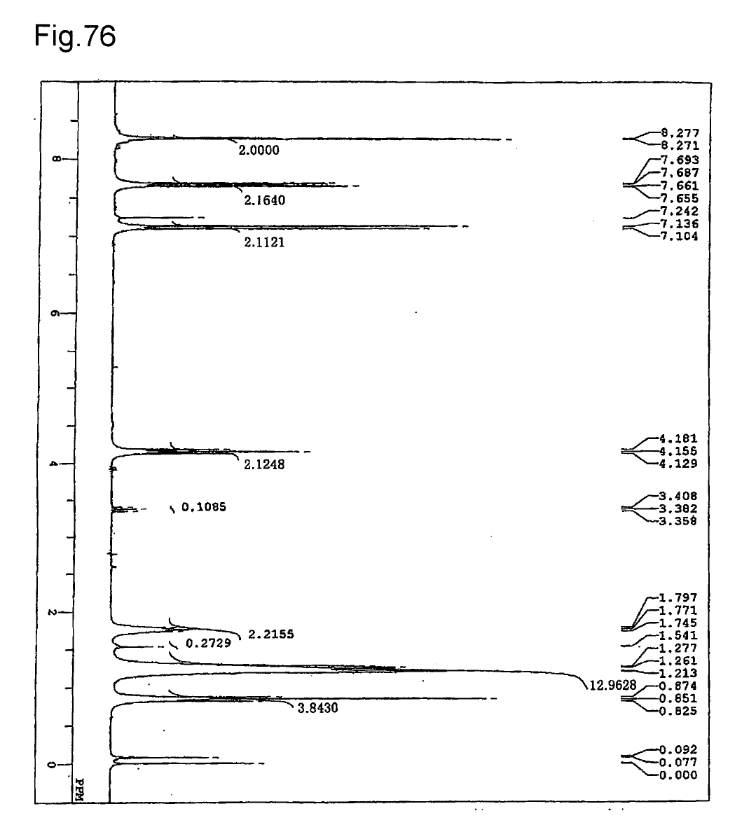

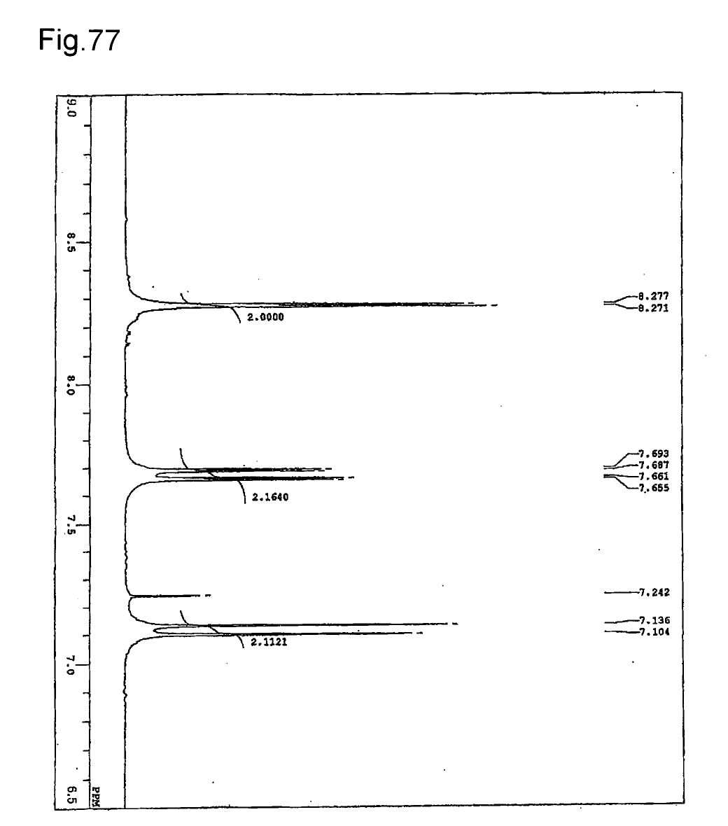

The 3,6-diiodo-9-oethylcarbazole thus obtained was subjected to ¹³C NMR and ¹H NMR measurements. FIG. 75 shows a graph obtained from the ¹³C NMR measurement, and FIGS. 76 and 77 show graphs obtained from the ¹H-NMR measurements. These measurement results are shown below.

¹H NMR (CDCl₃) 8.27 (d, J=1.6 Hz, 2H), 7.67 (d, J=8.4 Hz, 1.6 Hz, 2H), 7.12 (d, J=8.4 Hz, 2H), 4.16 (t, J=7.0 Hz, 2H), 1.80-1.75 (m, 2H), 1.28-1.21 (m, 10H), 0.85 (t, J=6.8 Hz, 3H);

¹³C NMR (CDCl₃) 139.15, 134.22, 129.06, 123.68, 110.70, 81.58, 43.15, 31.78, 29.33, 29.17, 28.82, 27.22, 22.65, 14.17.

### Synthesis of 3,6-Bis(triethoxysilyl)-9-oethylcarbazole

A mixture of 100 mg (0.19 mmol) of the 3,6-diiodo-9-oethylcarbazole obtained as described above and 3.6 mg (0.0094 mmol, 5 mol %) of [Rh(CH₃CN)₂(cod)]BF₄ was added with 4 ml of dimethylformamide (DMF) and 147 μl (1.13 mmol, 6 eq.) of triethylamine (TEA) under a nitrogen atmosphere. Then, the resultant mixture was stirred under a nitrogen atmosphere at room temperature for 30 minutes to obtain a mixed solution. Subsequently, the mixed solution thus obtained was added dropwise with 139 μl (0.75 mmol, 4 eq.) of triethoxysilane [(EtO)₃SiH] at room temperature, and stirred under a nitrogen atmosphere at 80° C. for 7 hours. Thereby, a reaction mixture (II) was obtained. Thereafter, the solvent in the reaction mixture (II) thus obtained was removed by distillation with a vacuum pump, and a residue was extracted with ether. After that, a salt thus formed was removed by filtering with celite. The solvent was removed by distillation from the organic phase with an evaporator to obtain a crude product (II). Then, the crude product (II) thus obtained was dissolved in 15 ml of ether, and purified by filtering the resultant through activated carbon (Kiriyama funnel, diameter: 1.5 cm, thickness: 5 mm). Thereby, a carbazole-silane compound was obtained (a yield of 80 mg and 70%).

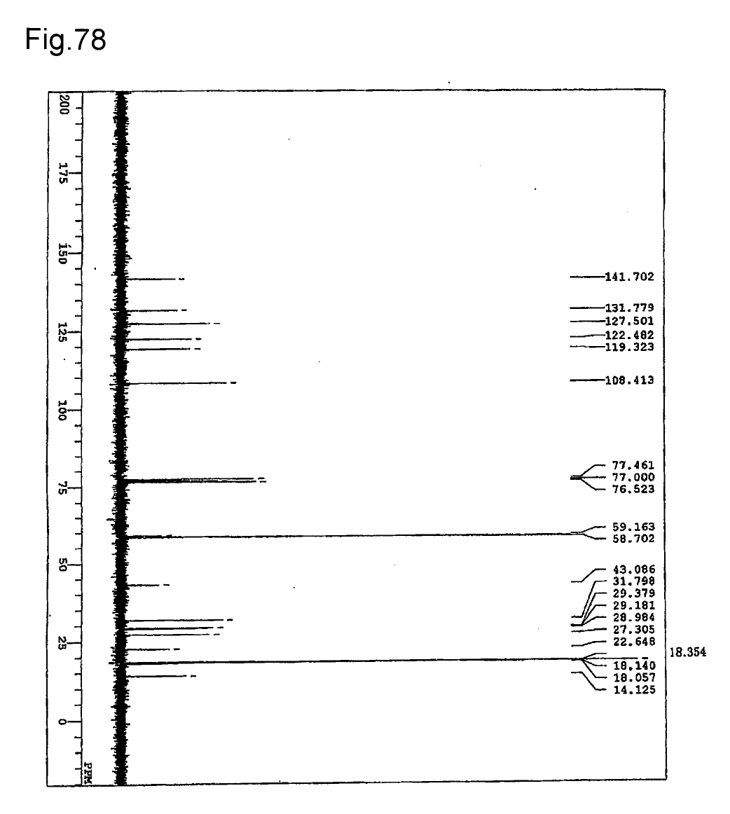

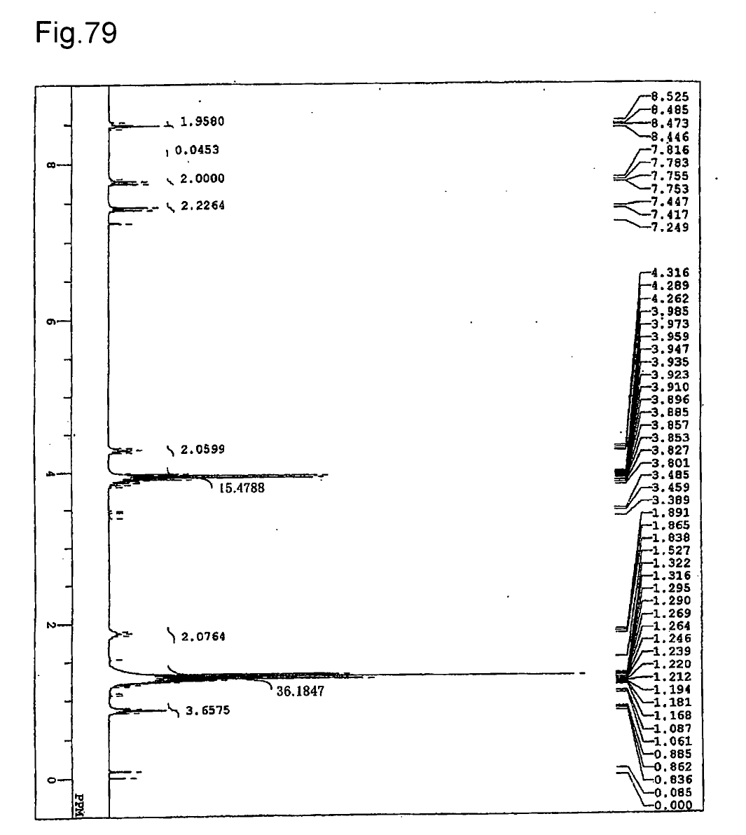

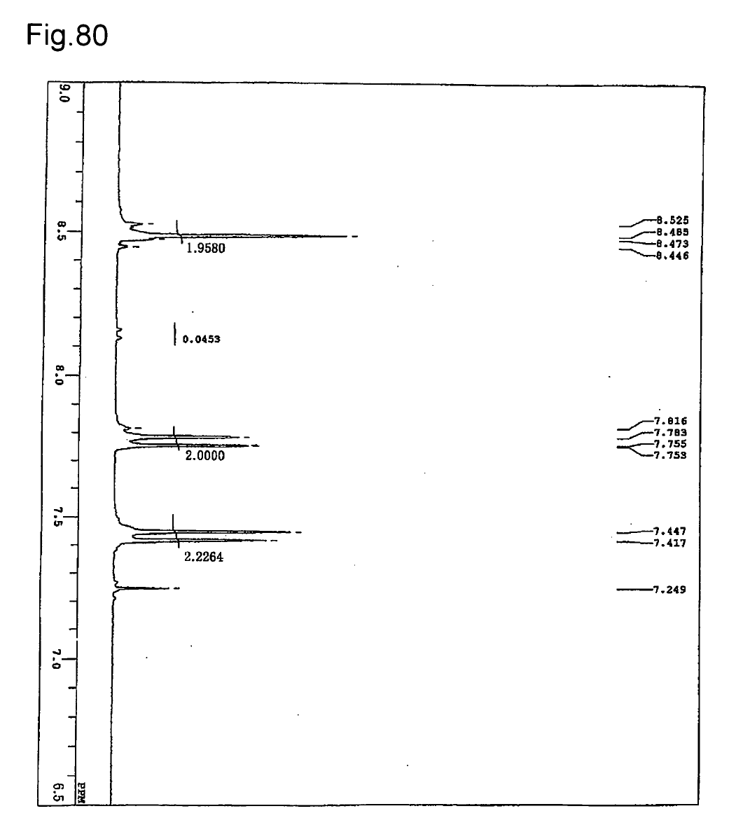

The carbazole-silane compound thus obtained was subjected to ¹³C NMR and ¹H NMR measurements. FIG. 78 shows a graph obtained from the ¹³C NMR measurement, and FIGS. 79 and 80 show graphs obtained from the ¹H-NMR measurements. These measurement results are shown below.

¹H NMR (CDCl₃) δ8.49 (s, 2H), 7.77 (d, J=8.1 Hz, 2H), 7.43 (d, J=8.1 Hz, 2H), 4.29 (t, J=7.3 Hz, 2H), 3.94 (q, J=7.3 Hz, 12H), 1.89-1.84 (m, 2H), 1.32-1.18 (m, 28H), 0.86 (t, J=7.3 Hz, 3H);

¹³C NMR (CDCl₃) δ141.70, 131.78, 127.50, 122.48, 119.32, 108.41, 58.70, 43.09, 31.80, 29.38, 29.18, 28.99, 27.31, 22.65, 18.35, 14.13.

Based on the NMR measurement results, it was confirmed that the carbazole-silane compound obtained in Example 21 was a carbazole-disilane compound expressed by the following general formula (99).

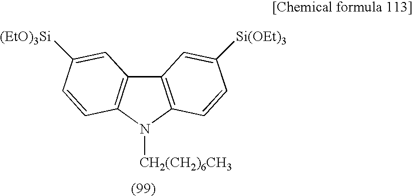

### Example 23

0.042 g of a triblock copolymer P123 ((EO)₂₀(PO)₇₀(EO)₂₀) was dissolved in a solution in which 22 μl of ion-exchanged water and 5 μl of a 2N hydrochloric acid aqueous solution had been added to 2 g of a mixed solvent of ethanol/THF (weight ratio of 1:1). Then, the solution was added with 0.05 g of BTECarb having a structure expressed by the following general formula (95) and stirred at room temperature for 20 hours or longer. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at room temperature for 24 hours or longer.

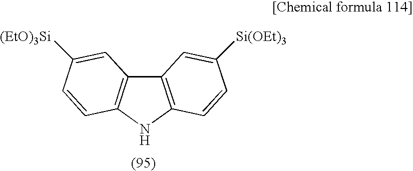

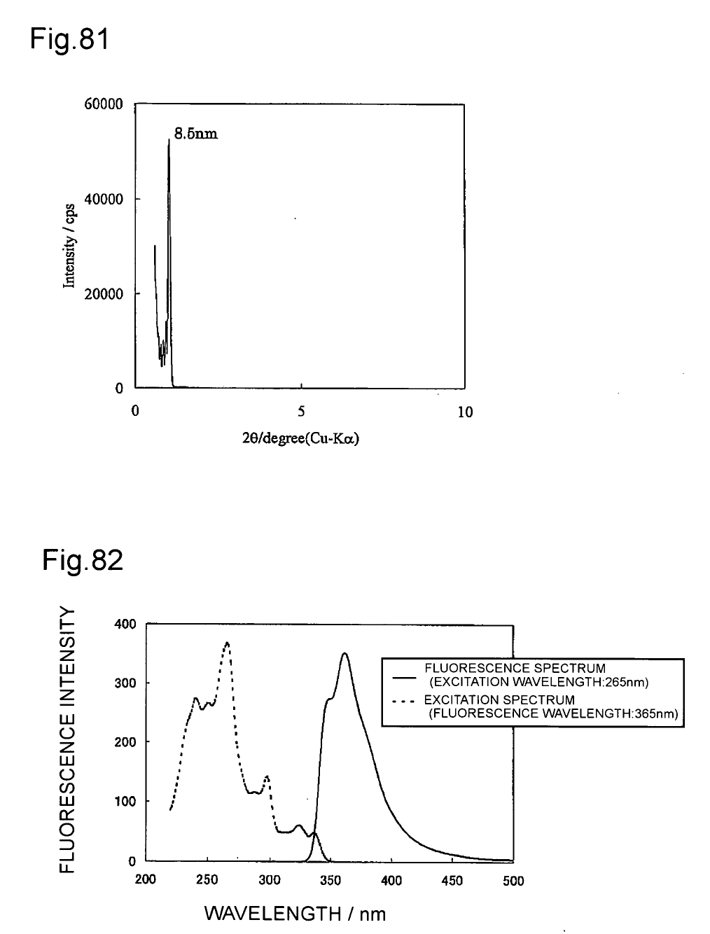

An X-ray diffraction pattern of the carbazole-silane-compound thin film (Carb-HMM-Acid-film) obtained in Example 23 and a fluorescence spectrum and an excitation spectrum thereof are respectively shown in FIGS. 81 and 82. In the X-ray diffraction pattern, a strong peak was observed at d=8.5 nm, and therefore it was confirmed that an ordered mesostructure was present (FIG. 81). Additionally, when a fluorescence spectrum was measured with an excitation wavelength of 265 nm, it was found that a strong emission was shown around 375 nm (FIG. 82).

### Example 24

0.05 g of BTECarb having a structure expressed by the following general formula (95) was added to a solution in which 22 μl of ion-exchanged water and 5 μl of a 2N hydrochloric acid aqueous solution had been added to 1 g of a mixed solvent of ethanol/THF (weight ratio of 1:1). Then, the solution was stirred at room temperature for 20 hours or longer. Thereby, a sol solution was obtained. Using this sol solution, a coating film (film thickness: 100 nm to 300 nm) was obtained by a spin coating method. In the coating conditions, the revolution speed was 4000 rpm, and the revolution time was 1 minute. The obtained film was dried at room temperature for 24 hours or longer.

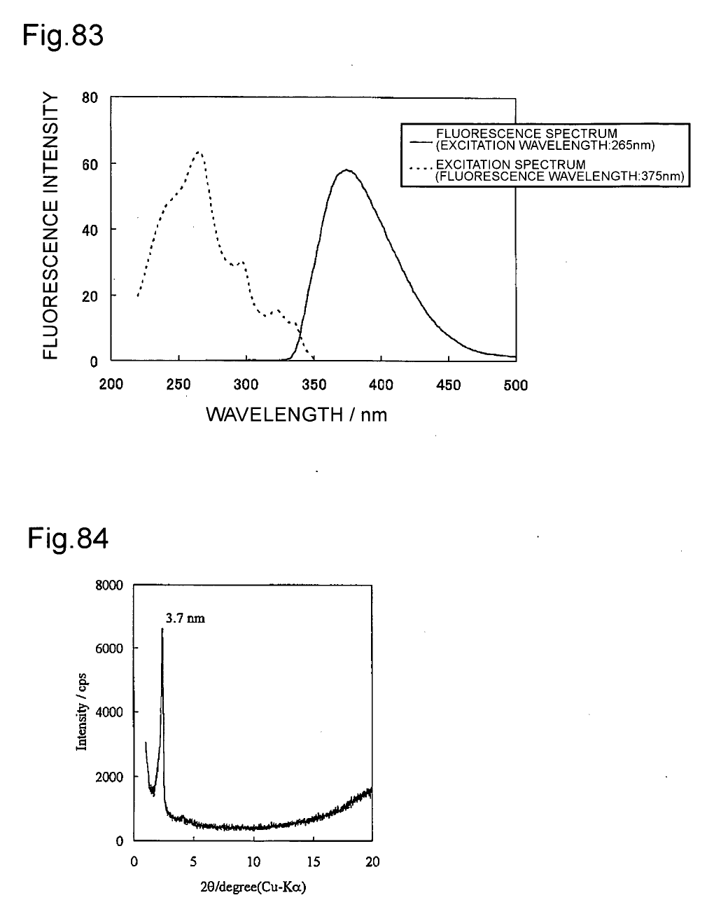

A fluorescence spectrum and an excitation spectrum of the carbazole-silane-compound thin film (Carb-Acid-film) obtained in Example 24 are shown in FIG. 83. When a fluorescence spectrum was measured with an excitation wavelength of 265 nm, it was found that a strong emission was shown around 375 nm (FIG. 83).

### Example 25

0.076 g of 1,12-bis(octadecyldimethylammonium)dodecan bromide (C₁₈₋₁₂₋₁₈) was dissolved in a water solution in which 6 g of ion-exchanged water and 100 ml of a 12N hydrochloric acid aqueous solution had been mixed. Then, the obtained solution was added with 0.1 g of the BTECarb having a structure expressed by the general formula (95) described above with vigorous stirring. The resultant solution was stirred at room temperature for 24 hours, and then heated at 60° C. for 24 hours. After being cooled to room temperature, the solution was filtered, washed, and dried to obtain a mesostructured powder.

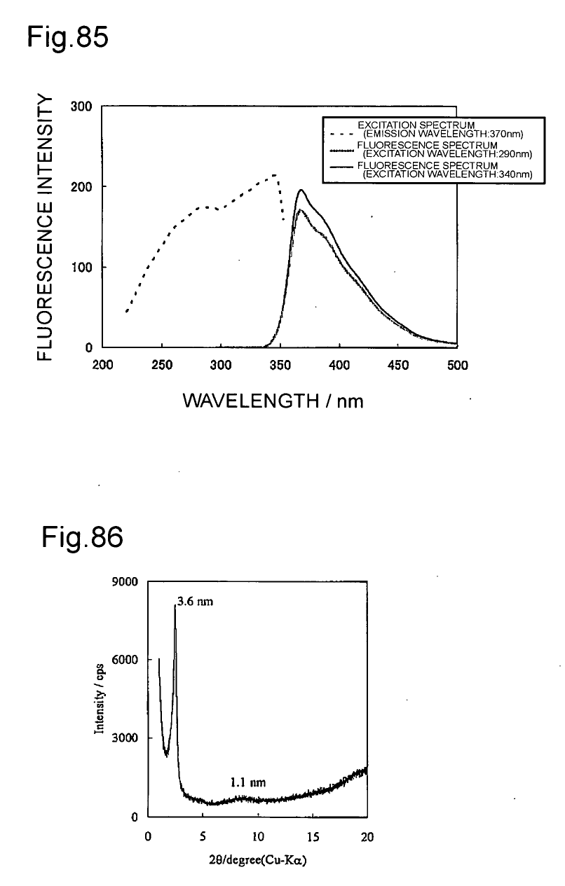

An X-ray diffraction pattern of the obtained powder (Carb-HMM-Acid) and a fluorescence spectrum and an excitation spectrum thereof are respectively shown in FIGS. 84 and 85. In the X-ray diffraction pattern, a peak was observed at d=3.7 nm, and therefore it was confirmed that an ordered mesostructure was present (FIG. 84). Additionally, when a fluorescence spectrum was measured with an excitation wavelength of 285 nm or 340 nm, it was found that a strong emission was shown around 365 nm (FIG. 85).

### Example 26

0.087 g of octadecyltrimethylammonium chloride was dissolved in a water solution in which 6 g of ion-exchanged water and 0.1 g of a 6 N NaOH solution were mixed together. Then, the obtained solution was added with 0.1 g of BTECarb having a structure expressed by the general formula (95) described above with vigorous stirring. The resultant solution was stirred at room temperature for 24 hours, and then heated at 60° C. for 20 hours. After being cooled to room temperature, the solution was filtered, washed, and dried to obtain a mesostructured powder.

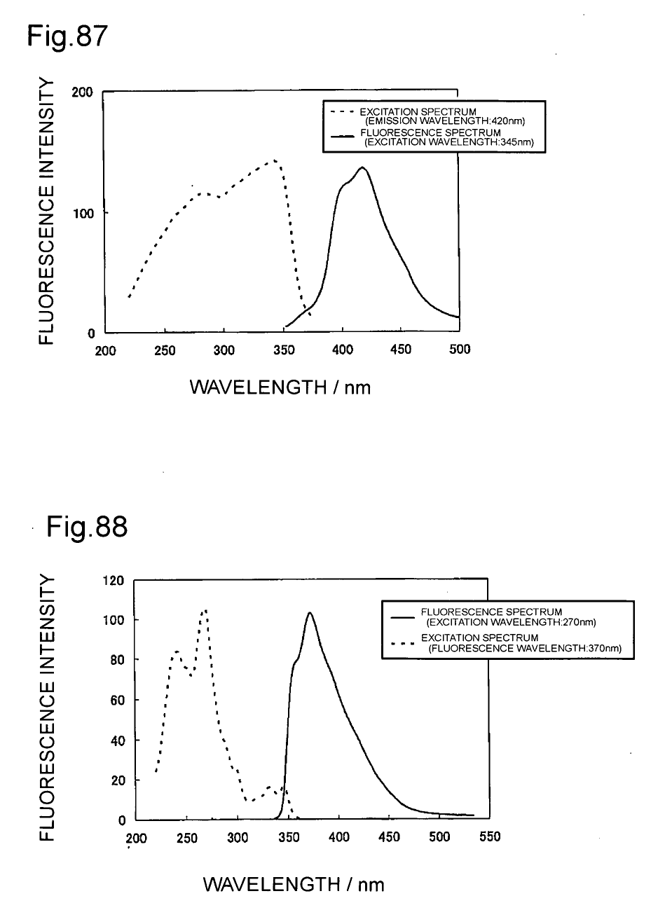

An X-ray diffraction pattern of the obtained powder (Carb-HMM-Base) and a fluorescence spectrum and an excitation spectrum thereof are respectively shown in FIGS. 86 and 87. In the X-ray diffraction pattern, a peak was observed at d=3.6 nm, and therefore it was confirmed that an ordered mesostructure was present (FIG. 86). Additionally, when a fluorescence spectrum was measured with an excitation wavelength of 345 nm, it was found that a strong emission was shown around 420 nm (FIG. 87).

### Example 27

A solution in which 1 g of a mixed solvent of ethanol/THF (weight ratio of 1:1) had been added with 22 μl of ion-exchanged water and 5 μl of a 2N hydrochloric acid aqueous solution was added with a solution in which 0.05 g of BTEMcarb having a structure expressed by the general formula (97) described above had been dissolved in 1 g of EtOH/THF (weight ratio of 1:1). Then, the resultant solution was stirred at room temperature for 20 hours or longer. Thereby, a sol solution was obtained. Using this sol solution, a coating film (film thickness: 100 nm to 200 nm) was obtained by a spin coating method. In the coating conditions, the revolution speed was 4000 rpm, and the revolution time was 30 seconds. The obtained film was dried at room temperature for 24 hours or longer.

A fluorescence spectrum and an excitation spectrum of the carbazole-disilane-compound thin film (Mcarb-Acid-film) obtained in Example 27 are shown in FIG. 88. When a fluorescence spectrum was measured with an excitation wavelength of 270 nm, it was found that a strong emission was shown around 370 nm (FIG. 88).

### Example 28

### synthesis of N,N′-Didodecyl-2,9-bis(triethoxysilyl)quinacridone)

### Synthesis of Dimethyl-2,5-bis[(4-bromophenyl)amino]cyclohexa-1,4-diene-1,4-dicarboxylate

9.12 g (40 mmol) of dimethyl-1,4-cyclohexanediiode-2,5-dicarboxylate was mixed with 200 ml of methanol to obtain a mixed solution. Then, the mixed solution was boiled. Note that, in such a boiling treatment, the mixed solution was added with 7.23 g (42 mmol) of 4-bromoaniline, and thereafter further added with 400 μl of concentrated hydrochloric acid. Subsequently, the mixed solution after the boiling treatment was refluxed under a nitrogen atmosphere for 3 hours, cooled to room temperature, and filtered. After that, a yellow precipitate thus obtained was washed with methanol, and dried under a reduced pressure. Thereby, dimethyl-2,5-bis[(4-bromophenyl)amino]cyclohexa-1,4-diene-1,4-dicarboxylate expressed by the following general formula (100) was obtained (a yield of 15.6 g and 73%).

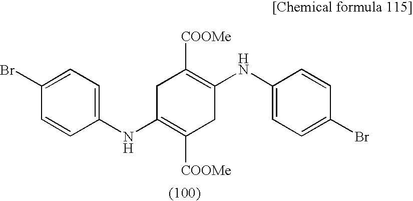

### Synthesis of 2,5-bis((4-bromophenyl)amino)terephthalic acid

8.04 g (15 mmol) of the dimethyl-2,5-bis[(4-bromophenyl)amino]cyclohexa-1,4-diene-1,4-dicarboxylate obtained as described above, 3.6 g (16 mmol) of 3-nitrobenzenesulfonic acid, 90 ml of ethanol, and 50 ml of a 1.0 M sodium hydroxide aqueous solution were refluxed under a nitrogen atmosphere overnight (10 hours) to obtain a mixed solution. Then, the mixed solution thus obtained was cooled to room temperature, and added with 120 ml of water. Subsequently, the mixed solution was adjusted to acidic with concentrated hydrochloric acid, and thereby a red precipitate was obtained. Thereafter, the mixed solution was filtered, and the obtained red precipitate was washed with water and dried under a reduced pressure. Thereby, 2,5-bis[(4-bromophenyl)amino]terephthalic acid expressed by the following general formula (101) was obtained (a yield of 7.0 g and 74%).

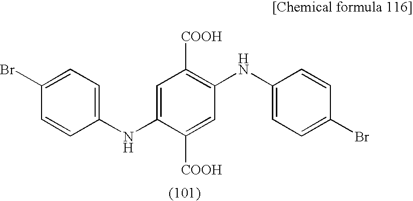

### Synthesis of 2.9-Dibromoquinacridone

2.0 g (4.0 mmol) of the 2,5-bis[(4-bromophenyl)amino]terephthalic acid obtained as described above and 20 g of polyphosphoric acid were stirred under a nitrogen atmosphere and a temperature condition of 150° C. for 3 hours to obtain a mixed solution. Then, the mixed solution thus obtained was cooled to room temperature (25° C.), and added with 80 ml of cold water to obtain a reddish violet precipitate. Subsequently, the mixed solution containing the precipitate was filtered, and the reddish violet precipitate thus obtained was washed with water and further with methanol, and dried under a reduced pressure. Thereby, 2.9-dibromoquinacridone expressed by the following general formula (102) was obtained (a yield of 1.76 g and 98%).

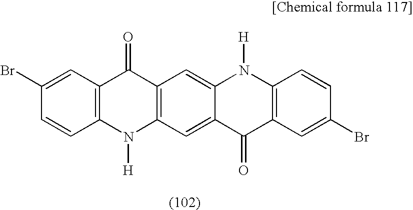

### Synthesis of N,N′-Didodecyl-2.9-Dibromoquinacridone

2.27 g (5.0 mmol) of the 2.9-dibromoquinacridone obtained as described above and 780 mg (19.5 mmol) of sodium hydride (60% suspension in oil) were stirred in 10 ml of anhydrous dimethylacetoamide under a nitrogen atmosphere until bubbling was ceased, and thereby a mixed solution was obtained. Then, the mixed solution thus obtained was stirred at 70° C. for 1 hour, and the color of the mixed solution turned to dark green. Subsequently, the mixed solution was added with 6.0 ml (25.0 mmol) of 1-bromododecan, stirred at 70° C. overnight, cooled to room temperature, and added with water. A precipitate thus obtained was filtered. Thereafter, the resultant was washed with hexane until the filtrate became colorless. A deposit on the filter paper surface was extracted with dichloromethane. After that, the resultant was dried with sodium sulfate to concentrate the solution. Thereby, N,N′-didodecyl-2.9-dibromoquinacridone expressed by the following general formula (103) was obtained (a yield of 1.05 g and 26%).

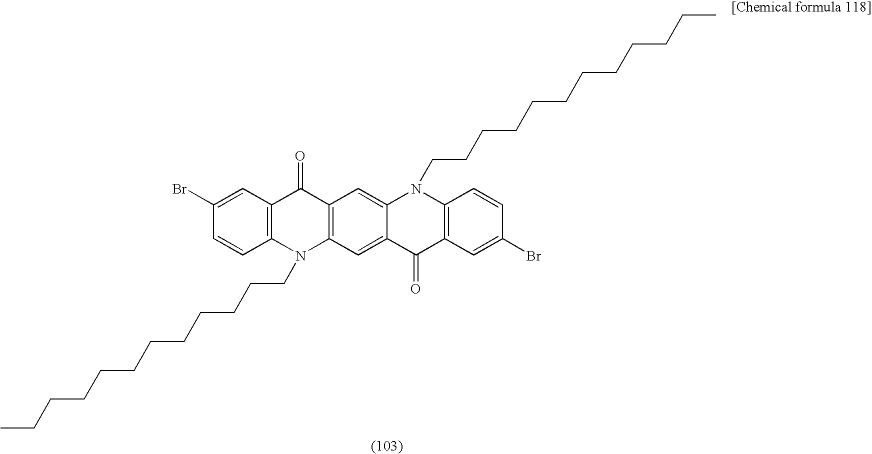

The N,N′-didodecyl-2.9-dibromoquinacridone thus obtained was subjected to ¹H NMR measurement. Note that, the NMR spectrum was measured with a JOEL JNM EX270 spectrometer (270 MHz for ¹H). Moreover, TMS was used as a reference for the chemical shifts in ¹H NMR. The measurement result is shown below.

¹H NMR (CDCl₃) δ8.62 (s, 2H), 8.56 (s, 2H), 7.78 (dd, J=4.6 Hz, 2H), 7.35 (d, J=4.6 Hz, 2H), 4.44 (t, J=7.8 Hz, 4H), 1.94 (t, 4H), 1.44 (m, 40H), 0.88 (t, J=6.8 Hz, 6H).

### Synthesis of N,N′-Didodecyl-2,9-bis(triethoxysilyl)quinacridone

A mixture of 1.64 mg (0.203 mmol) of the N,N′-didodecyl-2.9-dibromoquinacridone obtained as described above, 4.6 mg (0.012 mmol) of a [Rh(cod) (CH₃CN)₂]BF₄ complex, and 150 mg (0.406 mmol) of tetrabutylammoniumiodide was added with 4 ml of dimethylformamide (DMF) under a nitrogen atmosphere to obtain a mixed solution. Then, the mixed solution thus obtained was added with 0.17 ml (1.22 mmol) of triethylamine at room temperature. Subsequently, 0.15 ml (0.813 mmol) of triethoxysilane [(EtO)₃SiH] was added dropwise under a temperature condition of 0° C. Furthermore, the resultant mixed solution was stirred under a temperature condition of 80° C. for 2 hours. After the stirring, the DMF was removed with a vacuum pump from the mixed solution, and a residue was extracted with ether three times. After that, a salt thus formed was filtered with celite, and then concentrated. Thereby, a quinacridone-silane compound was obtained (a yield of 80 mg and 70%).

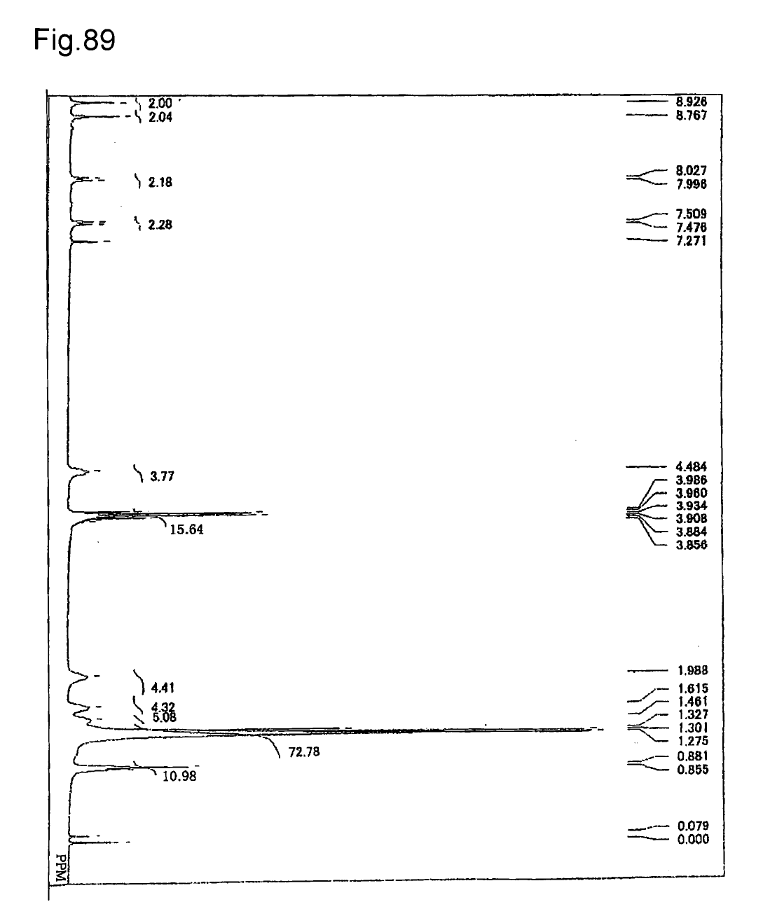

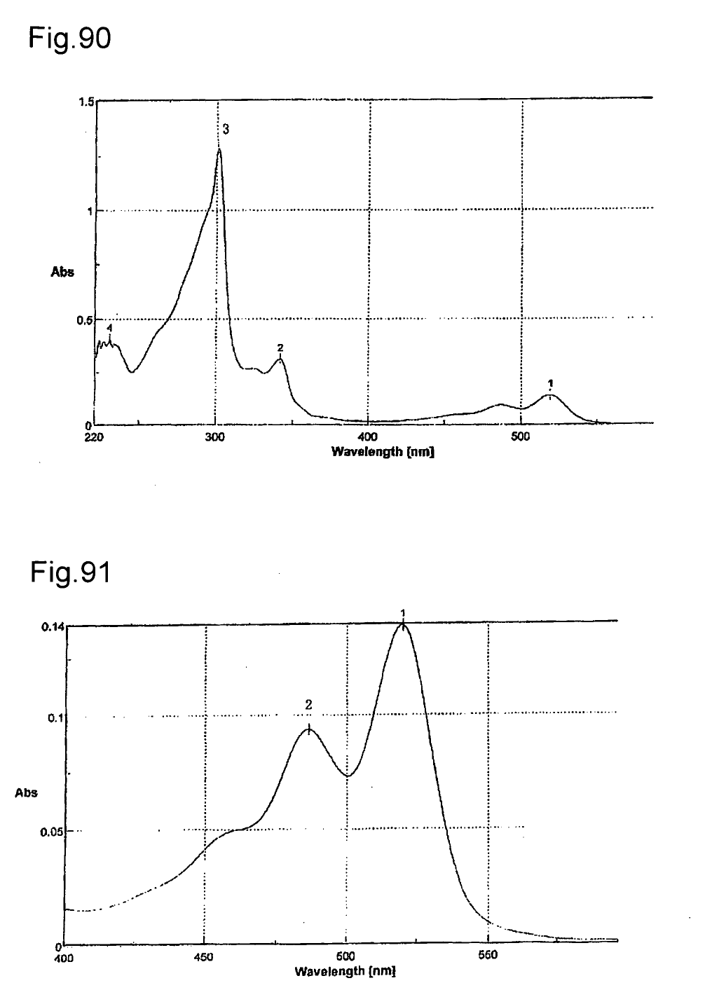

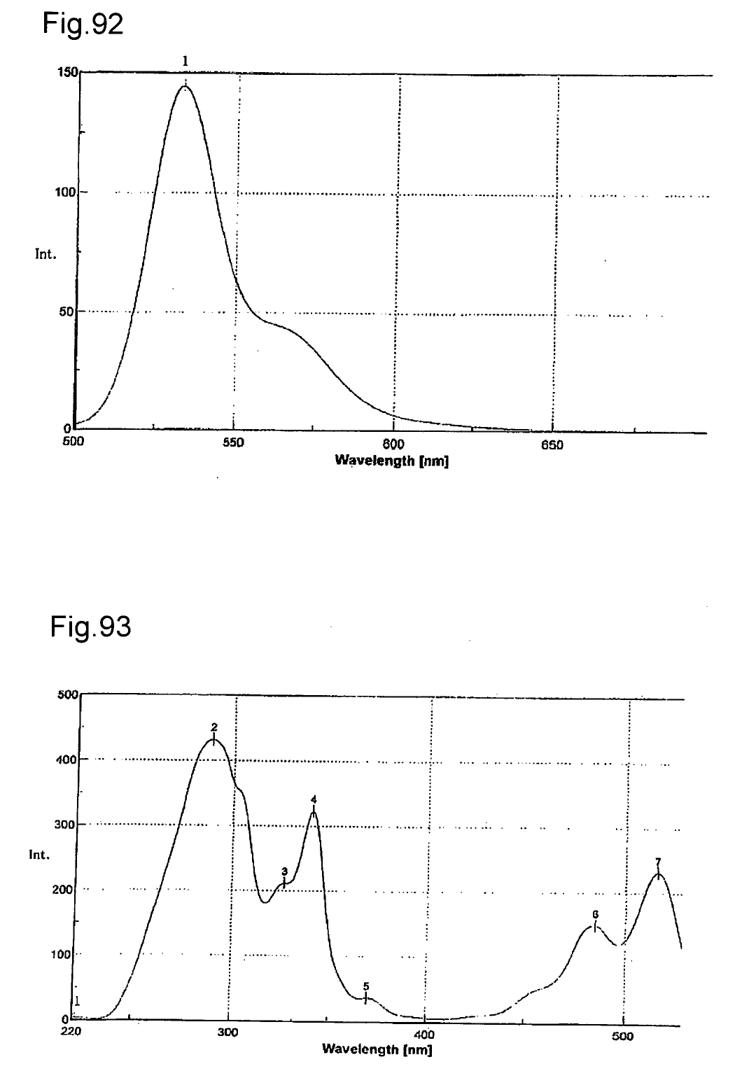

The quinacridone-silane compound thus obtained was subjected to ¹H NMR measurement. The obtained ¹H NMR measurement results are shown in FIG. 89 and below. Moreover, the UV spectra of the obtained quinacridone-silane compound are shown in FIGS. 90 and 91. Furthermore, a fluorescence spectrum (excitation wavelength: 486.5 nm) of the obtained quinacridone-silane compound (1×10⁻⁵ M) is shown in FIG. 92, and an excitation spectrum (measured wavelength: 533 nm) of the quinacridone-silane compound (1×10⁻⁵ M) is shown in FIG. 93. Note that, the NMR spectrum was measured with a JOEL JNM EX270 spectrometer (270 MHz for ¹H). Moreover, TMS was used as a reference for the chemical shifts in ¹H NMR.

¹H NMR (CDCl₃) δ8.93 (s, 2H), 8.77 (s, 2H), 8.01 (d, J=8.4 Hz, 2H), 7.50 (d, J=8.9 Hz, 2H), 4.48 (t, 4H), 3.92 (q, J=3.5 Hz, 12H), 1.99 (t, 4H), 1.62 (t, 4H), 1.37 (m, 54H), 0.88 (t, J=3.5 Hz, 6H).

Based on the NMR measurement results, it was confirmed that the quinacridone-silane compound obtained in Example 28 was a quinacridone-silane compound expressed by the following general formula (104).

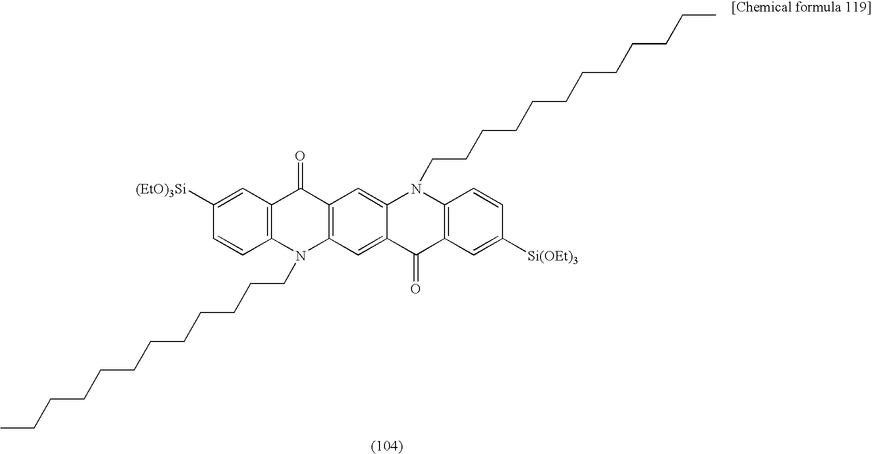

### Example 29

### Synthesis of 5,12-Bis(4-triethoxysilylphenyl)-6,11-diphenylnaphthacene)

### Synthesis of 6,11-diphenyl-5,12-naphthacenequinone

6.0 g (22.2 mmol) of 1,3-diphenylisobenzofuran in a powder form was added little by little to a solution, in which 3.51 g (22.2 mmol) of 1,4-naphthoquinone was dissolved in 120 mL of methylene chloride, to obtain a mixed solution. Then, the mixed solution thus obtained was stirred under a light-shielding condition at room temperature (25° C.) for 13 hours. Subsequently, this mixed solution was added with 170 mL of methylene chloride, cooled to −78° C. with dry ice/acetone, and slowly added dropwise with 24 mL (24 mmol) of a 1 M methylene chloride solution of boron tribromide (BBr₃). Thereafter, the resultant mixed solution was stirred under a temperature condition of −78° C. for 30 minutes, further stirred at room temperature (25° C.) for 2 hours, and then refluxed for 4 hours to obtain a reaction solution. Subsequently, the reaction solution thus obtained was poured into water and stirred. Thereafter, the aqueous phase and organic phase were separated, and the aqueous phase was extracted with chloroform. After that, the organic phase thus obtained was dried with anhydrous magnesium sulfate, and filtered. The filtrate was concentrated to obtain a residual solid. The residual solid was recrystallized by using a mixed solvent of chloroform and ethanol (chloroform/ethanol=1/1). Thereby, 6,11-diphenyl-5,12-naphthacenequinone expressed by the following general formula (105) was obtained (a yellow solid: a yield of 4.75 g and 52%).

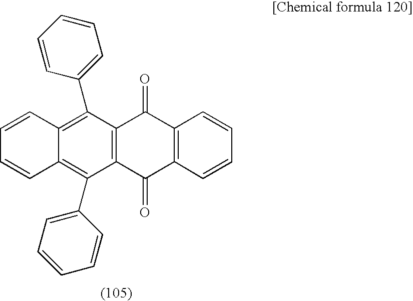

The 6,11-diphenyl-5,12-naphthacenequinone thus obtained was subjected to ¹H NMR measurement. Note that, the NMR spectrum was measured with a JOEL JNM EX270 spectrometer (270 MHz for ¹H). Moreover, TMS was used as a reference for the chemical shifts in ¹H NMR. The measurement result is shown below.

¹H NMR (CDCl₃) δ8.09 (dd, J=5.80, 3.33 Hz, 2H), 7.67 (dd, J=5.90, 2.60, 2H), 7.5-7.61 (m, 8H), 7.51 (dd, J=6.60, 3.30 Hz, 2H), 7.33-7.35 (m, 4H).

### Synthesis of 5,12-bis(4-methoxymethoxyphenyl)-6,11-diphenyl-5,12-naphthacenediol

20 mL of a THF solution containing 3.96 g (18.25 mmol) of 4-methoxymethoxybromobenzene, which was cooled to −78° C. with dry ice/acetone, was added dropwise with 7 mL (17.5 mmol) of a 2.5 M hexane solution of normal-butyllithium (n-BuLi), and the mixture was stirred for 30 minutes to obtain a solution. Then, the solution thus obtained was transferred, using a cannula, into 80 mL of a THF solution containing 1.50 g (3.65 mmol) of 6,11-diphenyl-5,12-naphthacenequinone, which was cooled to −78° C. with dry ice/acetone. Thereby, a mixed solution was obtained. Subsequently, the temperature of the mixed solution was gradually brought to room temperature with stirring for 24 hours. A saturated NH₄Cl aqueous solution was added to suppress the reaction. The aqueous phase in the mixed solution was extracted with ether. Thereafter, the organic phase thus obtained was washed with a saturated NH₄ Cl aqueous solution and a saturated NaCl aqueous solution, and dried with anhydrous magnesium sulfate. After that, magnesium sulfate was removed by filtration, and the filtrate was concentrated. Then, the filtrate thus obtained was added with hexane, and a precipitate thus formed was recovered by suction filtration. Subsequently, the obtained precipitate was thoroughly washed with hexane, and thus vacuum-dried. Thereby, 5,12-bis(4-methoxymethoxyphenyl)-6,11-diphenyl-5,12-naphthacenediol expressed by the following general formula (106) was obtained (a slightly yellowish white solid: a yield of 1.45 g and 58%).

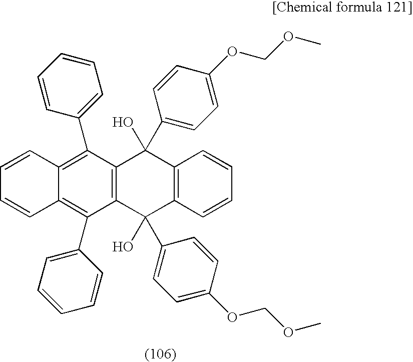

The 5,12-bis(4-methoxymethoxyphenyl-6,11-diphenyl-5,12-naphthacenediol thus obtained was subjected to ¹H NMR measurement. Note that, the NMR spectrum was measured with a JOEL JNM EX270 spectrometer (270 MHz for ¹H). Moreover, TMS was used as a reference for the chemical shifts in ¹H NMR. The measurement result is shown below.

¹H NMR (CDCl₃) δ7.72 (dd, J=5.60, 3.03 Hz, 2H), 7.57 (dd, J=6.39, 3.35 Hz, 2H), 7.49 (d, J=8.75 Hz, 4H), 7.29 (dd, J=6.39, 3.35 Hz, 2H), 7.14-7.25 (m, 10H), 6.95 (d, J=8.25 Hz, 2H), 6.72 (d, J=8.75 Hz, 4H), 5.10 (s, 4H), 3.44 (s, 6H).

### Synthesis of 5,12-Bis(4-hydroxyphenyl)-6,11-diphenylnaphthacene

1.5 g (2.18 mmol) of the 5,12-bis(4-methoxymethoxyphenyl-6,11-diphenyl-5,12-naphthacenediol obtained as described above was added with 150 mL of diethyl ether, and refluxed to obtain a mixture. Then, the mixture thus obtained was added dropwise with 16.5 mL of a 57 mass % hydrogen iodide (HI) aqueous solution, and refluxed for 30 minutes without any modification. Subsequently, the temperature of the mixture was returned to room temperature. A saturated sodium pyrosulfite (Na₂S₂O₅) aqueous solution was further added, and stirred. The aqueous phase and organic phase were separated, and the organic phase was extracted with ether. Thereafter, the organic phase thus obtained was dried with anhydrous magnesium sulfate. After that, the magnesium sulfate was removed by filtration, and the filtrate was concentrated. Thereby, a crude product (I) of 5,12-bis(4-hydroxyphenyl)-6,11-diphenylnaphthacene expressed by the following general formula (107) was obtained (a red solid: 1.3 g).

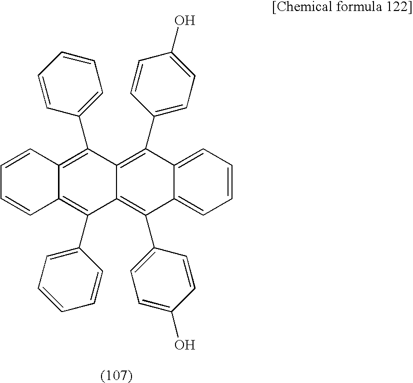

### synthesis of 5,12-Bis(4-trifluoromethylsulfonyloxyphenyl)-6,11-diphenylnaphthacene

1.7 g (3.01 mmol) of the crude product (I) of 5,12-bis(4-hydroxyphenyl)-6,11-diphenylnaphthacene obtained as described above was added with 180 mL of methylene chloride and 0.723 mL (9.0 mmol) of pyridine, and cooled to 0° C. to obtain a mixture. Then, the mixture was added dropwise with 2.02 mL (12 mmol) of trifluoromethanesulfonic anhydride, and stirred at room temperature for 17 hours to obtain a reaction mixed solution. Subsequently, the reaction mixed solution thus obtained was added with chloroform, and the aqueous phase and organic phase were separated. Thereafter, the organic phase was washed with a saturated NaHCO₃ aqueous solution and a saturated NaCl aqueous solution. The organic phase thus obtained was dried with anhydrous magnesium sulfate. After that, the magnesium sulfate was removed by filtration, and the filtrate was concentrated. Thereby, a crude product (II) was obtained. Then, the crude product (II) thus obtained was purified by silica gel column chromatography (hexane/chloroform=3/1), and thus 5,12-bis(4-trifluoromethylsulfonyloxyphenyl)-6,11-diphenylnaphthacene expressed by the following general formula (108) was obtained (a red solid: a yield of 0.45 g and 18%).

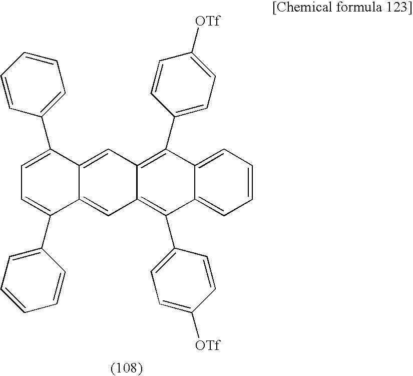

The 5,12-bis(4-trifluoromethylsulfonyloxyphenyl)-6,11-diphenylnaphthacene thus obtained was subjected to ¹H NMR measurement. Note that, the NMR spectrum was measured with a JOEL JNM EX270 spectrometer (270 MHz for ¹H). The measurement result is shown below.

¹H NMR (CDCl₃) δ7.40 (dd, J=7.05, 3.25 Hz, 2H), 7.21 (m, 4H), 7.13-7.18 (m, 8H), 6.93-6.99 (m, 8H), 6.89 (d, J=7.10 Hz, 4H).

### Synthesis of 5,12-Bis(4-triethoxysilylphenyl)-6,11-diphenylnaphthacene

A mixture of 340 mg (0.41 mmol) of the 5,12-bis(4-trifluoromethylsulfonyloxyphenyl)-6,11-diphenylnaphthacene obtained as described above, 15.2 mg (0.04 mmol, 10 mol %) of a [Rh(cod) (CH₃CN)₂]BF₄ complex, and 303 mg (0.82 mmol) of normal-tetrabutylnickel (n-Bu₄NI) was added with DMF (6 mL) and TEA (0.34 mL, 2.46 mmol, 6 eq.) after argon substitution, and thereby a mixed solution was obtained. Subsequently, the mixed solution thus obtained was cooled to 0° C., and added with 0.303 mL (1.64 mmol, 4 eq.) of triethoxysilane. Thereafter, the resultant mixed solution was stirred under a temperature condition of 80° C. for 24 hours to obtain a suspension. Then, after the DMF in the suspension thus obtained was removed with a vacuum pump, the suspension was extracted with ether three times, and filtered with celite to obtain a filtrate. Then, the filtrate thus obtained was filtered through activated carbon (powder). Thereby, a rubrene-silane compound was obtained (a red amorphous solid: 300 mg, 85%).

The rubrene-silane compound thus obtained was subjected to ¹H NMR measurement. Note that, the NMR spectrum was measured with a JOEL JNM EX270 spectrometer (270 MHz for ¹H). Moreover, TMS was used as a reference for the chemical shifts in ¹H NMR. The obtained H NMR measurement result is shown below.

¹H NMR (CDCl₃) δ7.4-7.24 (m, 10H), 6.96 (m, 8H), 6.89 (d, J=7.10 Hz, 4H), 6.63 (dd, J=31.15, 8.85 Hz, 4H), 3.87 (q, J=7.05 Hz, 12H), 1.24 (t, J=7.15 Hz, 18H).

Based on the NMR measurement result, it was confirmed that the rubrene-silane compound obtained in Example 29 was a rubrene-disilane compound (5,12-bis(4-triethoxysilylphenyl)-6,11-diphenylnaphthacene) expressed by the following general formula (109).

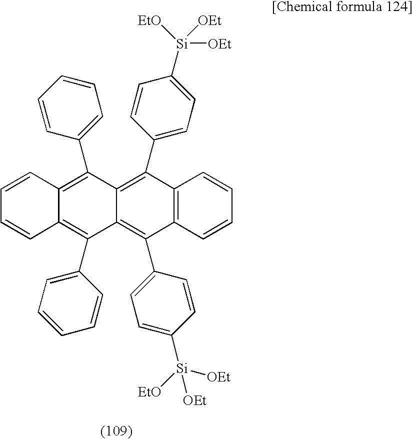

### Example 30

### synthesis of 1,4-Dihexyloxy-2,5-bis(4-triethoxysilylphenylethenyl)benzene)

### Synthesis of 1,4-Dihexyloxy-2,5-bis(4-iodophenylethenyl)benzene

A mixture of 1.00 g (2.99 mmol) of 2,5-dihexyloxyterephthalaldehyde and 2.20 g (6.2 mmol) of diethyl p-iodobenzylphosphonate was added with 100 mL of dehydrated THF, and cooled to 0° C. Then, a mixture of 1.68 g (15 mmol) of tert-butyloxypotassium (tert-BuOK) and 40 mL of THF was slowly added thereto, and thereby a mixed solution was obtained. Subsequently, the mixed solution was stirred at room temperature for 16 hours. Thereafter, approximately 150 mL of water was added thereto, and stirred. A pale yellow solid formed in the mixed solution was recovered by suction filtration. After that, the pale yellow solid thus obtained was washed with water and ethanol, and vacuum-dried. Thereby, 1,4-dihexyloxy-2,5-bis(4-iodophenylethenyl)benzene expressed by the following general formula (110) was obtained (a single-yellow solid: a yield of 1.82 g and 83%).

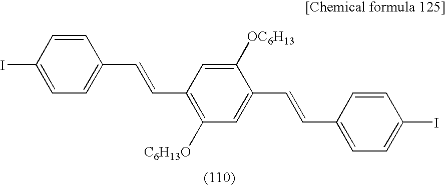

The 1,4-dihexyloxy-2,5-bis(4-iodophenylethenyl)benzene thus obtained was subjected to ¹H NMR measurement. Note that, the NMR spectrum was measured with a JOEL JNM EX270 spectrometer (270 MHz for ¹H). Moreover, TMS was used as a reference for the chemical shifts in ¹H NMR. The obtained H NMR measurement result is shown below.

¹H NMR (CDCl₃) δ7.67 (d, J=8.45 Hz, 4H), 7.46 (d, J=16.45 Hz, 2H), 7.26 (d, J=8.45 Hz, 4H), 7.09 (s, 2H), 7.04 (d, J=16.45 Hz, 2H), 4.04 (t, J=6.35 Hz, 4H), 1.86 (m, 4H), 1.30-1.60 (m, 12H), 0.92 (t, J=7.05, 6H)<

### synthesis of 1,4-Dihexyloxy-2,5-bis(4-triethoxysilylphenylethenyl)benzene

A mixture of 1.50 g (2.04 mmol) of the 1,4-dihexyloxy-2,5-bis(4-iodophenylethenyl)benzene obtained as described above and 38 mg (0.1 mmol, 5 mol %) of a [Rh(cod) (CH₃CN)₂]BF₄ complex was added with 40 mL of dist.DMF and 1.67 mL (12 mmol, 6 eq.) of dist.TEA after argon substitution, and thereby a mixed solution was obtained. Subsequently, the mixed solution thus obtained was cooled to 0° C., and added with 1.51 mL (8.16 mmol, 4 eq.) of triethoxysilane. Thereafter, the resultant mixed solution was stirred at 80° C. for 3 hours, and a suspension was obtained. After that, the DMF was removed with a vacuum pump from the suspension thus obtained, and a residue was extracted with ether three times, and filtered with celite to obtain a filtrate. Then, the filtrate thus obtained was further filtered through activated carbon (powder) for concentration thereof, and further filtrated through cotton fibers to obtain a yellow-green viscous liquid. Subsequently, the yellow-green viscous liquid thus obtained was left standing for 3 days or longer, and gradually crystallized. Thereby, a 1,4-dihexyloxy-2,5-phenylethenylbenzene-silane compound was obtained (a yield of 1.20 g and 73%).

The 1,4-dihexyloxy-2,5-phenylethenylbenzene-silane compound thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown below.

¹H NMR (CDCl₃) δ7.66 (d, J=8.45 Hz, 4H), 7.55 (m, 6H), 7.13 (m, 4H), 4.06 (t, J=6.35 Hz, 4H), 3.89 (q, J=7.00 Hz, 12H), 1.87 (m, 4H), 1.30-1.60 (m, 12H), 1.26 (t, J=6.95 Hz, 18H), 0.93 (t, J=7.05, 6H);

¹³C NMR (CDCl₃) δ151.2, 139.8, 135.2, 129.8, 128.6, 126.9, 125.9, 110.7, 69.6, 58.7, 31.6, 29.5, 25.9, 22.6, 18.4, 14.0.

Based on the NMR measurement results, it was confirmed that the 1,4-dihexyloxy-2,5-phenylethenylbenzene-silane compound obtained in Example 30 was a 1,4-dihexyloxy-2,5-phenylethenylbenzene-disilane compound (1,4-dihexyloxy-2,5-bis(4-triethoxysilylphenylethenyl)benzene) expressed by the following general formula (111).

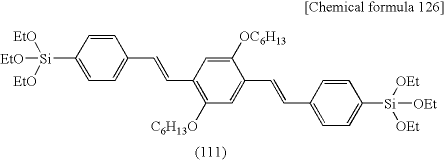

### Example 31

### synthesis of tris(4-triethoxysilylphenyl)amine)

### Synthesis of tris(4-iodophenyl)amine

A mixture of 5.3 g (14.3 mmol, 3.5 eq.) of bis(pyridine)iodonium tetrafluoroborate (IPy₂BF₄) and 1 g (4.1 mmol) of triphenylamine was added with 60 ml of dichloromethane (dist.CH₂Cl₂) under a nitrogen atmosphere to obtain a mixed solution. Then, the mixed solution thus obtained was cooled to 0° C., and added dropwise with 900 μl (4.1 mmol, 1 eq.) of trifluoromethanesulfonic acid (TfOH). The resultant mixed solution was stirred under a nitrogen atmosphere at room temperature for 21 hours to obtain a reaction mixture. Subsequently, the reaction mixture thus obtained was added with a saturated sodium thiosulfate (Na₂S₂O₃) aqueous solution to suppress the reaction. Thereafter, the aqueous phase in the reaction solution was extracted with dichloromethane. Thereby, the organic phase containing the reddish-brown reaction mixture was obtained. After that, the organic phase thus obtained was washed with a saturated NaCl solution, dried with Na₂SO₄, filtered, and concentrated to obtain a crude product (2.9714 g). Then, the crude product thus obtained was separated and purified by silica gel column chromatography (hexane:ethyl acetate=5:1). Thereby, tris(4-iodophenyl)amine was obtained (a yield of 2.507 g and 99%).

The tris(4-iodophenyl)amine thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown below.

¹H NMR (CDCl₃) δ7.54 (d, J=8.9 Hz, 6H), 6.81 (d, J=8.9 Hz, 6H);

¹³C NMR (CDCl₃) δ146.5, 138.4, 126.0, 86.6.

In addition, the following reaction formula (H) shows an outline of the synthesis method for the tris(4-iodophenyl)amine.

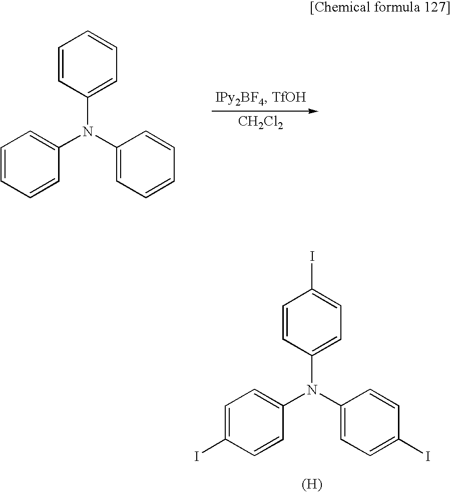

### Tris(4-triethoxysilylphenyl)amine

A mixture of 100 mg (0.16 mmol) of the tris(4-iodophenyl)amine obtained as described above, 5.4 mg (0.014 mmol, 9 mol %) of a [Rh(CH₃CN)₂(cod)]BF₄ complex, and 195 mg (0.48 mmol, 3 eq.) of PPh₃MeI was added dropwise with 4 ml of DMF, 201 μl (1.45 mmol, 9 eq.) of triethylamine, and 178 μl (0.96 mmol, 6 eq.) of triethoxysilane (EtO)₃SiH). Then, the resultant mixture was stirred under a nitrogen atmosphere at 80° C. for 1 hour to obtain a reaction mixture. Subsequently, a solvent in the reaction mixture thus obtained was removed by distillation with a vacuum pump, and a residue was extracted with ether. Thereafter, a salt thus formed was removed by filtering with celite. After that, the solvent was removed by distillation from the organic phase with an evaporator to obtain a crude product (128.4 mg). Then, the crude product thus obtained was dissolved in 15 ml of ether, and purified by filtering the resultant through activated carbon (Kiriyama funnel, thickness: 7 mm). Thereby, a triphenylamine-silane compound was obtained (118.4 mg, 100%).

The obtained triphenylamine-silane compound was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown below.

¹H NMR (CDCl₃) δ7.54 (d, J=8.6 Hz, 6H), 7.09 (d, J=8.6 Hz, 6H), 3.89 (q, J=7.0 Hz, 18H), 1.26 (t, J=7.0 Hz, 27H);

¹³C NMR (CDCl₃) δ148.9, 135.8, 124.7, 123.5, 58.7, 18.2.

Based on the NMR measurement results, it was confirmed that the triphenylamine-silane compound obtained in Example 31 was tris(4-triethoxysilylphenyl)amine.

In addition, the following reaction formula (1) shows an outline of the synthesis method for the tris(4-triethoxysilylphenyl)amine.

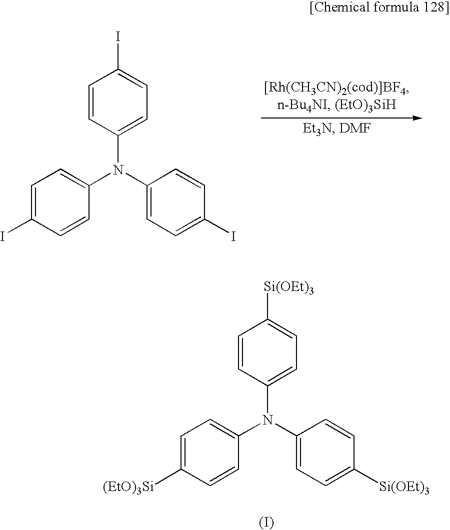

### Example 32

### synthesis of tris(4-diallylethoxysilylphenyl)amine)

242 mg (0.33 mmol) of the tris(4-triethoxysilylphenyl)amine obtained in Example 31 was added with 5 ml of ether under a nitrogen atmosphere, and further added dropwise with 4 ml (12 eq.) of allylmagnesium bromide (1M ether solution) under a temperature condition of 0° C. to obtain a reaction mixture. Then, the reaction mixture thus obtained was stirred under a nitrogen atmosphere at room temperature for 20 hours, and cooled (quenched) with H₂O. The aqueous phase in the reaction mixture was added with 10 mass % HCl to adjust the pH to 4. Subsequently, the organic phase was separated therefrom, and the aqueous layer was extracted with ether. The collected organic phase was washed with a saturated NaHCO₃ aqueous solution and a saturated NaCl aqueous solution, dried with magnesium sulfate, filtered, and concentrated to obtain a crude product (214 mg). Then, the crude product thus obtained was separated and purified by preparative thin-layer chromatography (PTLC: hexane/ethyl acetate=10/1). Thereby, a triphenylamine-silane compound was obtained (a yield of 80 mg and 34%).

The triphenylamine-silane compound thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown below.

¹H NMR (CDCl₃) δ7.46 (d, J=8.4 Hz, 6H), 7.09 (d, J=8.4 Hz, 6H), 5.93-5.77 (m, 6H), 5.00-4.90 (m, 12H), 3.79 (q, J=7.0 Hz, 6H), 1.93 (d, J=7.8 Hz, 12H), 1.22 (t, J=7.0 Hz, 9H);

¹³C NMR (CDCl₃) δ148.5, 135.1, 133.3, 129.0, 123.4, 114.7, 59.2, 21.3, 18.4.

Based on the NMR measurement results, it was confirmed that the triphenylamine-silane compound obtained in Example 32 was tris(4-diallylethoxysilylphenyl)amine.

In addition, the following reaction formula (J) shows an outline of the synthesis method for the tris(4-triethoxysilylphenyl)amine.

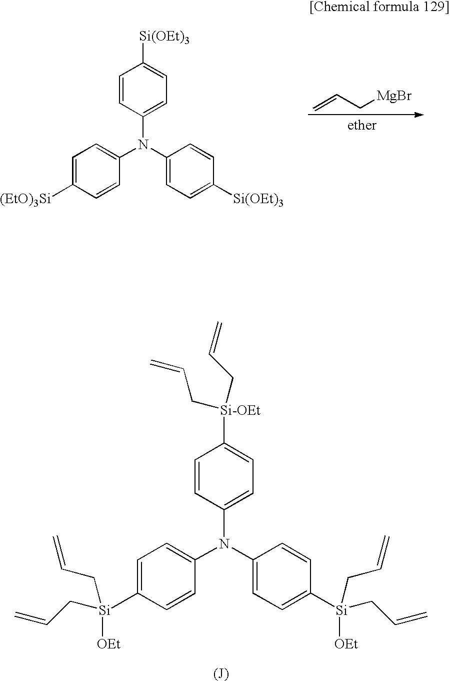

### Example 33

### synthesis of 3,6-bis(diallylethoxysilyl)carbazole)

902 mg (1.83 mmol) of the 3,6-bis(triethoxysilyl)carbazole obtained as in Example 20 was added with 1 ml of dist.ether, and further added dropwise with 11 ml (11 mmol, 6 eq.) of allylmagnesium bromide under a nitrogen atmosphere and a temperature condition of 0° C. to obtain a reaction mixture. Then, the reaction mixture thus obtained was stirred under a nitrogen atmosphere at room temperature for 18 hours, and added with 10 mass % HCl to adjust the pH of the aqueous phase in the reaction mixture to 4. Subsequently, the organic phase was separated from the reaction mixture, and the aqueous phase was extracted with ether. Thereafter, the obtained organic phase was washed with a saturated NaHCO₃ solution and a saturated NaCl solution, dried with anhydrous magnesium sulfate. After that, the magnesium sulfate was removed by filtration, and the filtrate was concentrated. Thereby, a crude product was obtained (945.3 mg). Then, the crude product thus obtained was separated and purified by silica gel column chromatography (hexane:ethyl acetate=20:1). Thereby, a carbazole-silane compound was obtained (a yield of 695.9 mg and 80%).

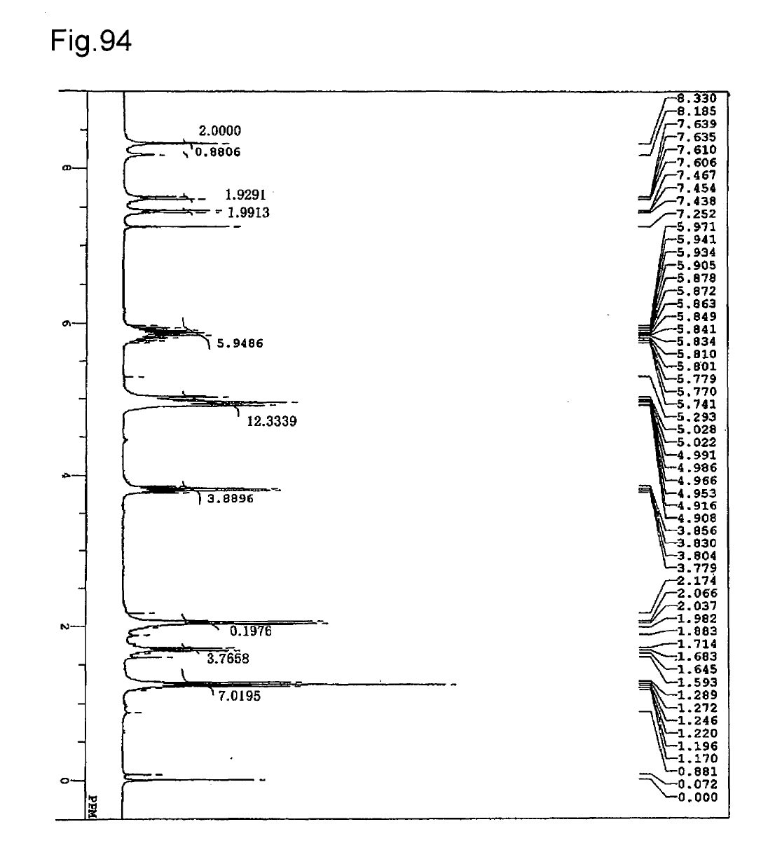

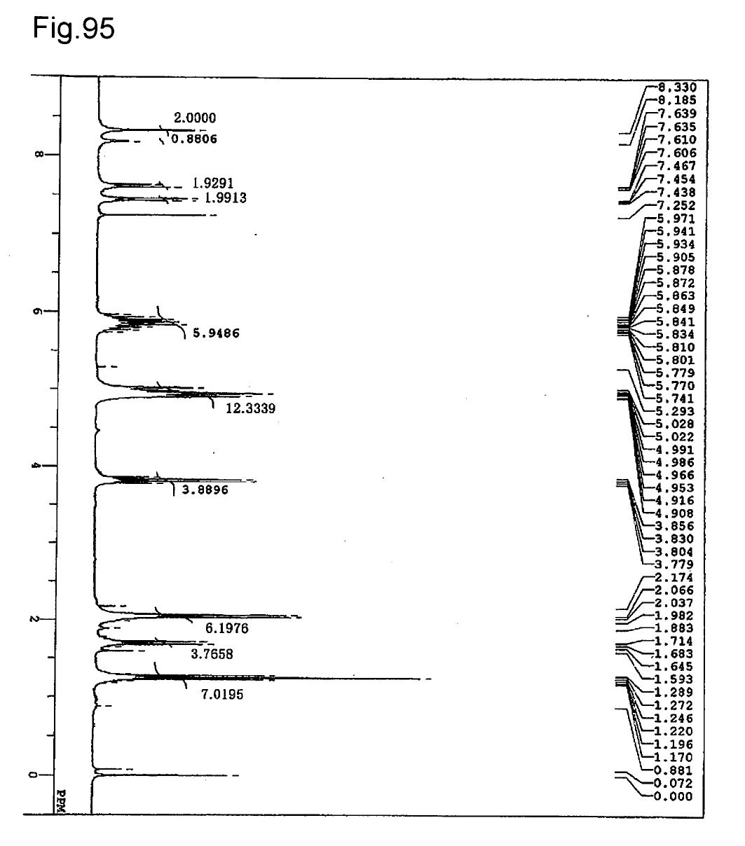

The carbazole-silane compound thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown in FIG. 94 (¹H NMR), FIG. 95 (¹H NMR), and below.

¹H NMR (CDCl₃) δ8.34 (d, J=1.1 Hz, 2H), 7.62 (dd, J=1.1 Hz, 8.1 Hz, 2H), 7.41 (d, J=8.1 Hz, 2H), 6.00-5.82 (m, 4H), 5.04-4.87 (m, 8H), 3.82 (q, J=7.0 Hz, 4H), 2.05 (d, J=7.8 Hz, 8H), 1.25 (t, J=7.0 Hz, 6H);

¹³C NMR (CDCl₃) δ140.4, 133.5, 131.4, 126.4, 124.7, 122.9, 114.6, 110.3, 59.3, 21.6, 18.4.

Based on the NMR measurement results, it was confirmed that the carbazole-silane compound obtained in Example 33 was 3,6-bis(diallylethoxysilyl)carbazole.

In addition, the following reaction formula (K) shows an outline of the synthesis method for the 3,6-bis(diallylethoxysilyl)carbazole.

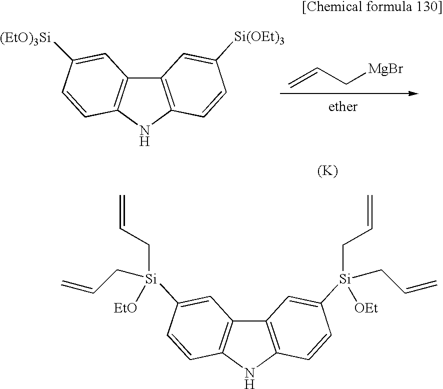

### Example 34

### synthesis of 3,6-bis(diallylethoxysilyl)-9-methylcarbazole)

1.5 g (2.97 mmol) of the 3,6-bis(triethoxysilyl)-9-methylcarbazole obtained as in Example 21 was added with 30 ml of dist.ether, and further added dropwise with 26.7 ml (9 eq.) of allylmagnesium bromide under a nitrogen atmosphere at 0° C. to obtain a reaction mixture. Then, the reaction mixture thus obtained was stirred under a nitrogen atmosphere at room temperature for 16 hours, and added with 10 mass % HCl to adjust the pH of the aqueous phase of the reaction mixture to 4. Subsequently, the organic phase was separated from the reaction mixture, and the aqueous phase was extracted with ether. The obtained organic phase was washed with a saturated NaHCO₃ aqueous solution and a saturated NaCl aqueous solution, dried with anhydrous magnesium sulfate. Thereafter, the magnesium sulfate was removed by filtration, and the filtrate was concentrated. Thereby, a carbazole-silane compound was obtained (a yield of 1.45 g and 99%).

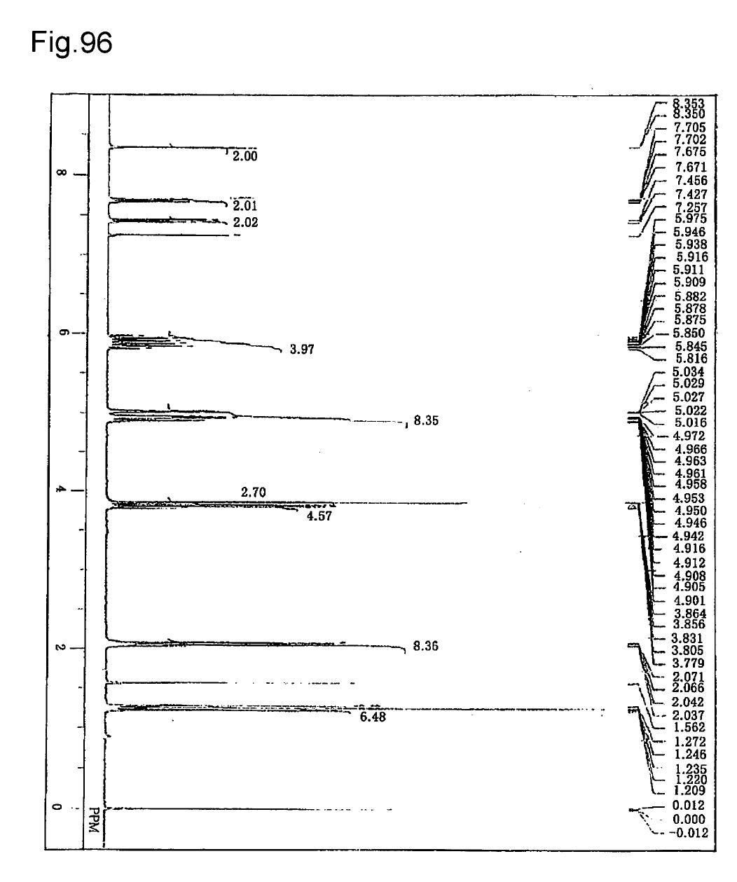

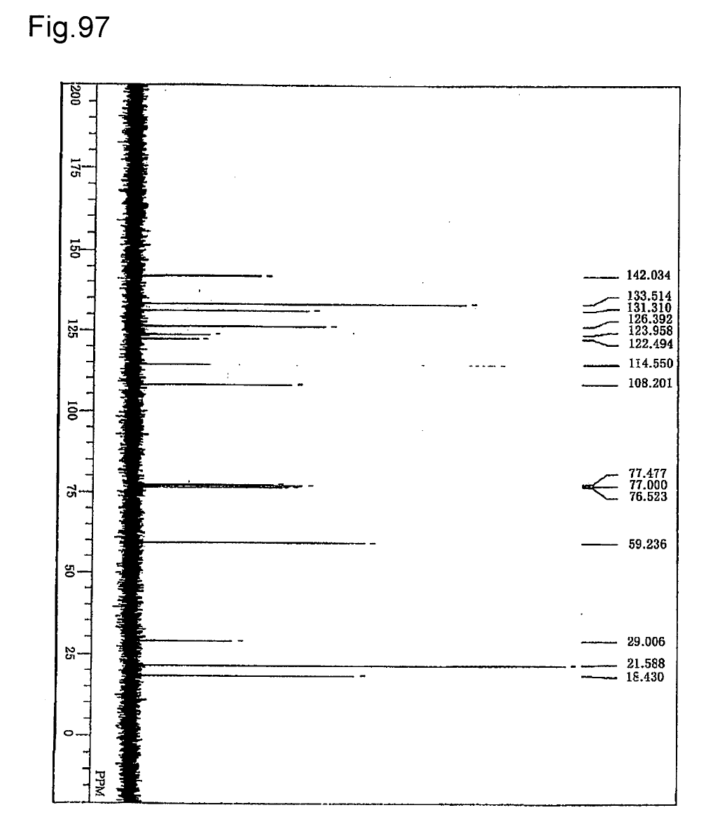

The carbazole-silane compound thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown in FIG. 96 (¹H NMR) and FIG. 97 (¹³C NMR), and below.

¹H NMR (CDCl₃) δ8.35 (d, J=0.8 Hz, 2H), 7.69 (dd, J=0.8 Hz, 8.1 Hz, 2H), 7.44 (d, J=8.1 Hz), 5.98-5.82 (m, 4H), 5.03-4.90 (m, 8H), 3.89 (s, 3H), 3.82 (q, J=7.0 Hz, 4H), 2.06 (d, J=7.8 Hz, 8H), 1.24 (t, J=7.0 Hz, 6H);

¹³C NMR (CDCl₃) δ142.3, 133.5, 131.3, 126.4, 124.0, 122.5, 114.6, 108.2, 59.2, 29.0, 21.6, 18.4.

Based on the NMR measurement results, it was confirmed that the carbazole-silane compound obtained in Example 34 was 3,6-bis(diallylethoxysilyl)-9-methylcarbazole.

In addition, the following reaction formula (L) shows an outline of the synthesis method for the 3,6-bis(diallylethoxysilyl)-9-methylcarbazole.

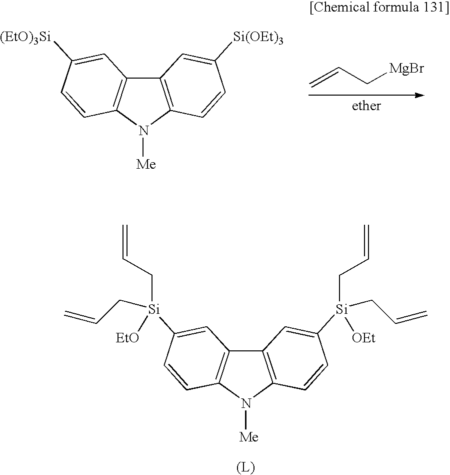

### Example 35

### 2,7-bis(diallylethoxysilyl)fluorene)

1058 mg (2.2 mmol) of the 2,7-bis(triethoxysilyl)fluorene obtained as in Example 1 was added dropwise with 12.9 ml (12.9 mmol, 6 eq.) of allylmagnesium bromide under a nitrogen atmosphere at 0° C. to obtain a reaction mixture. Then, the reaction mixture thus obtained was stirred under a nitrogen atmosphere at room temperature for 18 hours, and added with 10 mass % HCl to adjust the pH of the aqueous phase of the reaction mixture to 4. Subsequently, the organic phase was separated from the reaction mixture, and the aqueous phase was extracted with ether. The obtained organic phase was washed with a saturated NaHCO₃ aqueous solution and a saturated NaCl aqueous solution, dried with anhydrous magnesium sulfate. Thereafter, the magnesium sulfate was removed by filtration, and the filtrate was concentrated to obtain a crude product. The crude produce thus obtained was separated and purified by silica gel column chromatography (hexane:ethyl acetate=20:1). Thereby, a fluorene-silane compound was obtained (a yield of 829.3 mg and 81%).

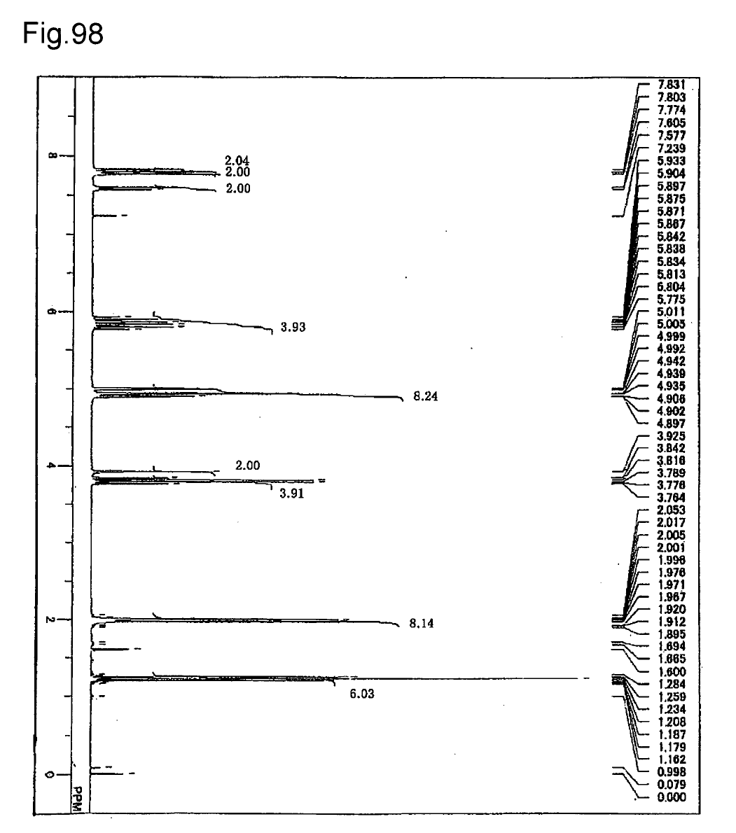

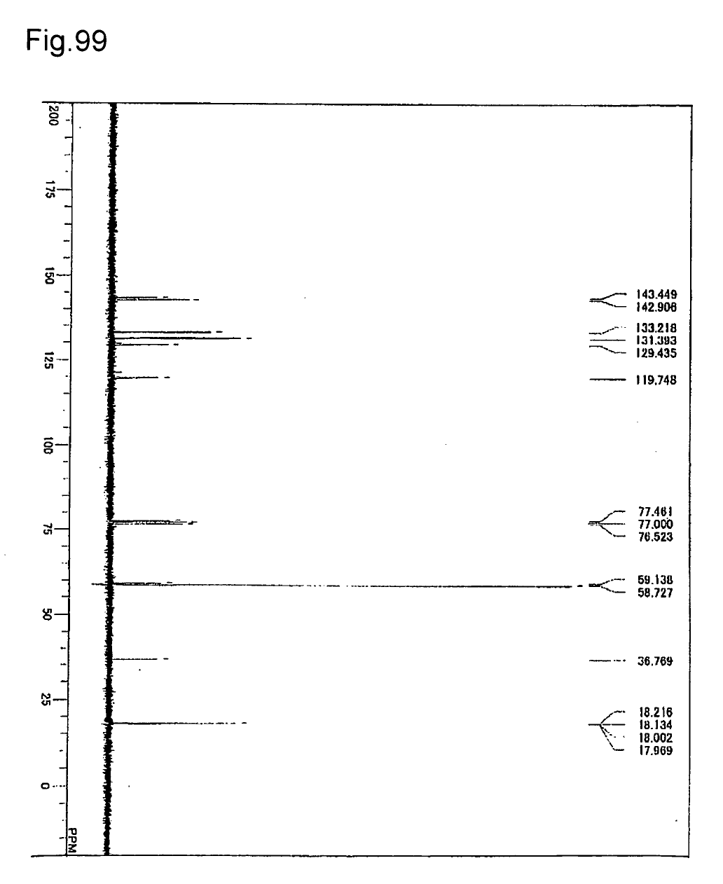

The fluorene-silane compound thus obtained was subjected to ¹³C NMR and ¹H NMR measurements. Note that, the NMR spectra were measured with a JOEL JNM EX270 spectrometer (270 MHz for ¹H). Moreover, TMS was used as a reference for the chemical shifts in ¹H NMR, and CDCl₃ was used as a reference for the chemical shifts in ¹³C NMR. The measurement results are shown in FIG. 98 (¹H NMR), FIG. 99 (¹³C NMR), and below.

¹H NMR (CDCl₃) δ7.82 (d, J=7.6 Hz, 2H), 7.77 (s, 2H), 7.59 (d, J=7.6 Hz, 2H), 5.93-5.78 (m, 4H), 5.01-4.90 (m, 8H), 3.93 (s, 2H), 3.80 (q, J=7.3 Hz, 4H), 1.99 (d, J=8.1 Hz, 8H), 1.23 (t, J=7.3 Hz, 6H);

¹³C NMR (CDCl₃) δ143.0, 142.8, 133.7, 133.2, 132.5, 130.6, 119.6, 114.7, 59.3, 36.9, 21.4, 18.4.

Based on the NMR measurement results, it was confirmed that the fluorene-silane compound obtained in Example 35 was 2,7-bis(diallylethoxysilyl)fluorene.

In addition, the following reaction formula (M) shows an outline of the synthesis method for the 2,7-bis(diallylethoxysilyl)fluorene.

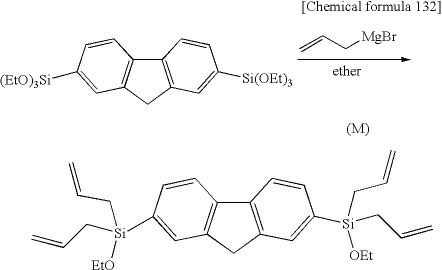

### INDUSTRIAL APPLICABILITY

As has been described, the present invention makes it possible to provide a bridged organosilane, which has a large complex organic group, and which is useful for the synthesis of a mesoporous silica and a light-emitting material, and to provide a production method of the bridged organosilane. The bridged organosilane of the present invention is accordingly a disilane compound having a large complex organic group, such as fluorene and pyrene, and therefore useful as a bridged organosilane for the synthesis of a mesoporous silica material and a light-emitting material.

1. A bridged organosilane, expressed by the following general formula (1): 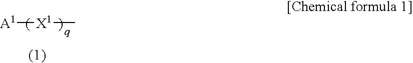
wherein, in the formula (1),
q represents an integer in a range from 2 to 4,
X¹— represents a substituent selected from the group consisting of substituents expressed by the following general formulae (2) to (5): 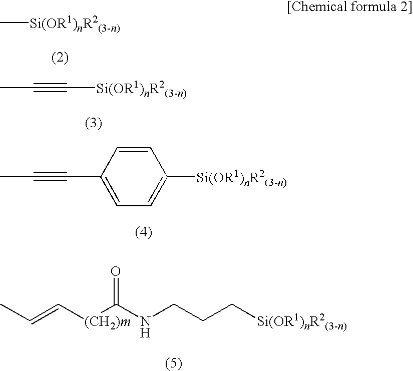
(in the formulae (2) to (5), R¹ represents any one of alkyl groups having 1 to 5 carbon atoms, R² represents an allyl group, n represents an integer in a range from 0 to 3, and m represents an integer in a range from 0 to 6), and
A¹ represents one organic group selected from the group consisting of organic groups expressed by the following general formula (6): 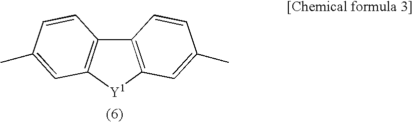
{in the formula (6), Y¹< represents a substituent selected from the group consisting of substituents expressed by the following general formulae (7) to (12): 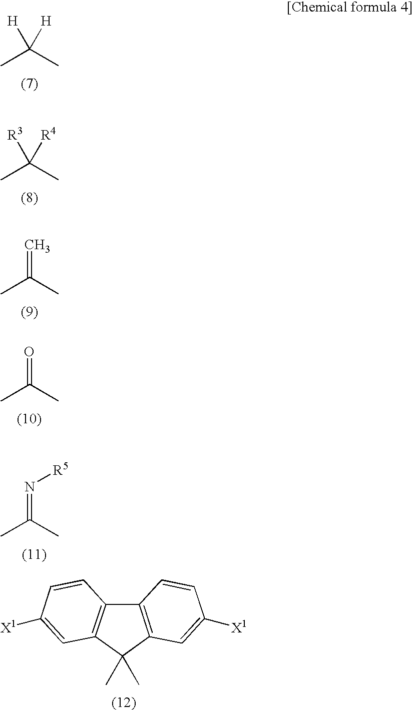
(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (12), X¹— represents a substituent selected from the group consisting of substituents expressed by the formulae (2) to (5))},
organic groups expressed by the following general formulae (13) and (14): 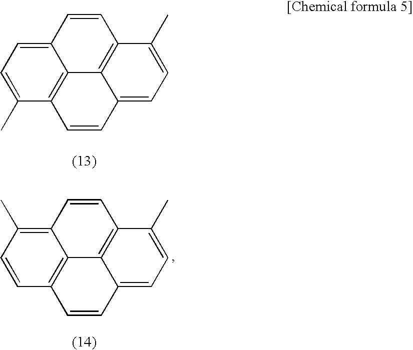
organic groups expressed by the following general formulae (15) to (17): 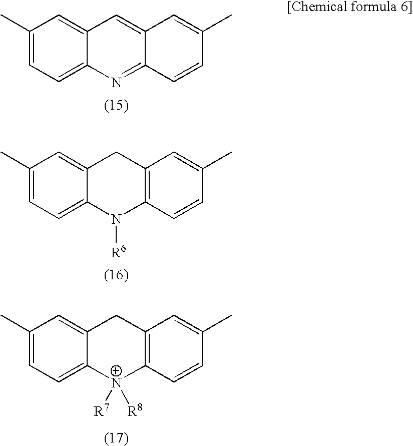
(in the formula (16), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (17), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms),
an organic group expressed by the following general formula (18): 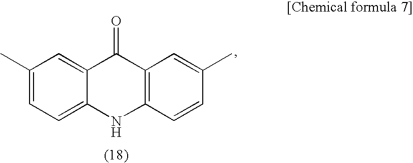
an organic group expressed by the following general formula (19): 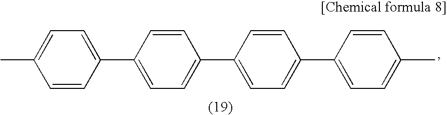
organic groups expressed by the following general formulae (20) and (21): 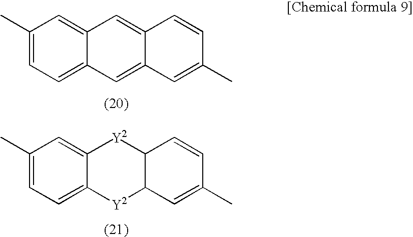
{in the formula (21), Y²< represents a substituent expressed by any one of the following general formulae (10) and (11): 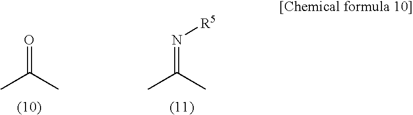
(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms)},
organic groups expressed by the following general formulae (22) and (23): 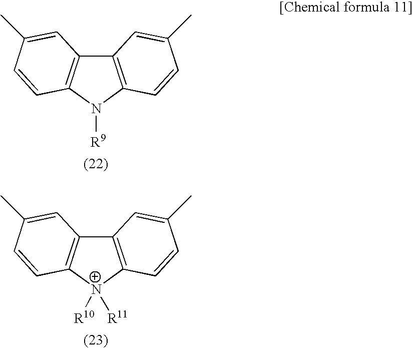
(in the formula (22), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (23), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formula (24): 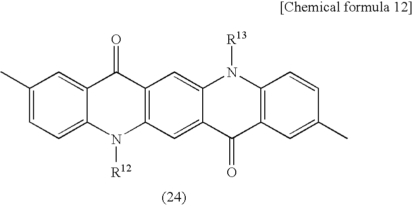
(in the formula (24), R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formulae (25) and (26): 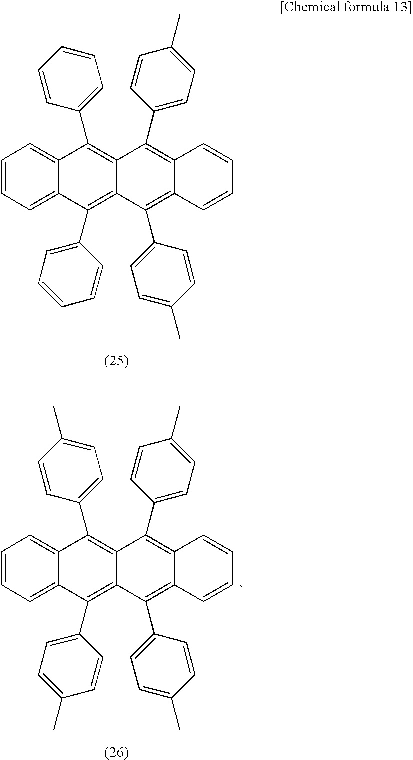
organic groups expressed by the following general formula (27): 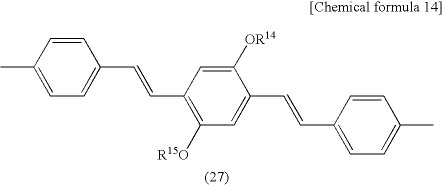
(in the formula (27), R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms), and
an organic group expressed by the following general formula (28):

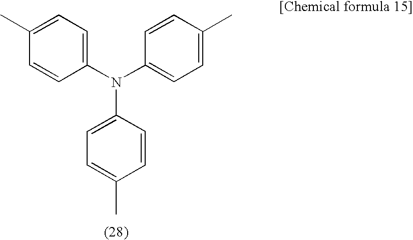

2. The bridged organosilane according to claim 1, which is a fluorene-silane compound expressed by the following general formula (29): 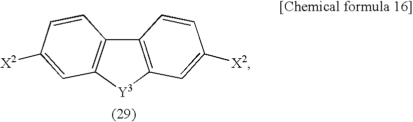
wherein, in the formula (29),
X²— represents a substituent selected from the group consisting of substituents expressed by the following general formulae (2) to (4): 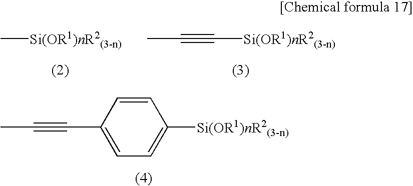
(in the formulae (2) to (4), R¹ represents any one of alkyl groups having 1 to 5 carbon atoms, R² represents an allyl group, and n represents an integer in a range from 0 to 3), and
Y³< represents a substituent selected from the group consisting of substituents expressed by the following general formulae (7) to (11) and (30): 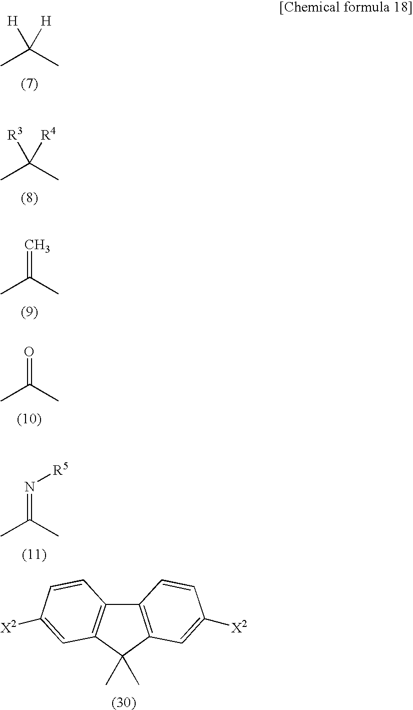
(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (30), X²— represents a substituent selected from the group consisting of substituents expressed by the formulae (2) to (4)).

3. The bridged organosilane according to claim 1, which is a pyrene-silane compound expressed by any one of the following general formulae (31) and (32): 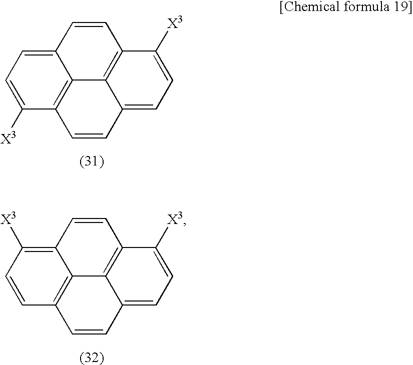
wherein, in the formulae (31) and (32),
X³— represents a substituent expressed by the following general formula (2):
[Chemical formula 20]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3).

4. The bridged organosilane according to claim 1, which is an acridine-silane compound expressed by any one of the following general formulae (33), (34) and (35): 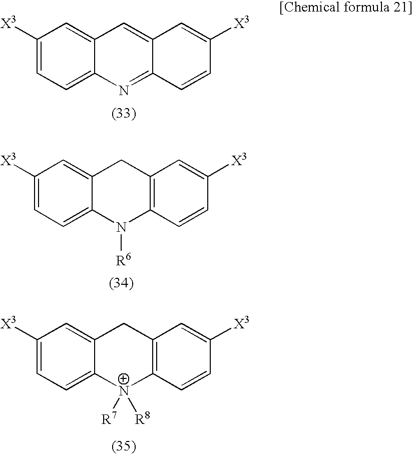
wherein, in the formulae (33) to (35), X³— represents a substituent expressed by the following general formula (2):
[Chemical formula 22]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3); in the formula (34), R⁶ represents any one of a hydrogen atom, alkyl groups having 1 to 22 carbon atoms, perfluoroalkyl groups having 1 to 22 carbon atoms, and aryl groups having 6 to 8 carbon atoms, and; in the formula (35), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms.

5. The bridged organosilane according to claim 1, which is an acridone-silane compound expressed by the following general formula (36): 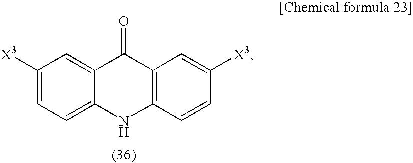
wherein, in the formula (36), X³— represents a substituent expressed by the following general formula (2):
[Chemical formula 24]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3).

6. The bridged organosilane according to claim 1, which is a quaterphenyl-silane compound expressed by the following general formula (37): 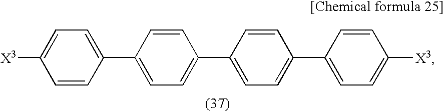
wherein, in the formula (37), X³— represents a substituent expressed by the following general formula (2):
[Chemical formula 26]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3).

7. The bridged organosilane according to claim 1, which is any one of an anthracene-silane compound, an anthraquinone-silane compound, and an anthraquinonediimine-silane compound, expressed by any one of the following general formulae (38) and (39): 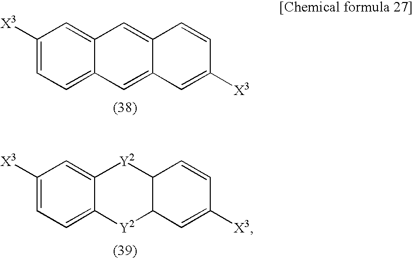
wherein, in the formulae (38) and (39), X³— represents a substituent expressed by the following general formula (2):
[Chemical formula 28]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3; and in the formula (39), Y²< represents a substituent expressed by any one of the following general formulae (10) and (11): 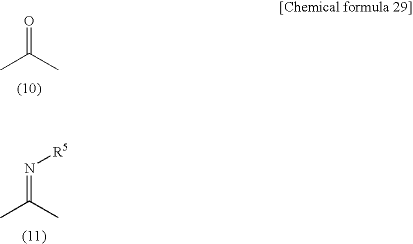
(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms).

8. The bridged organosilane according to claim 1, which is a carbazole-silane compound expressed by any one of the following general formulae (40) and (41): 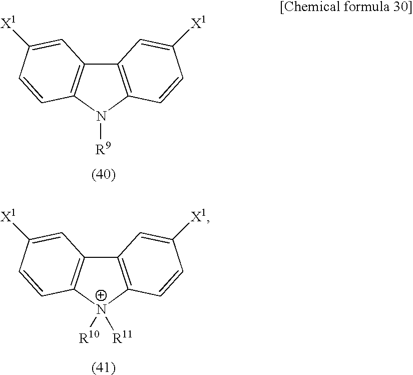
wherein, in the formulae (40) and (41), X¹— represents a substituent selected from the group consisting of substituents expressed by the following general formulae (2) to (5): 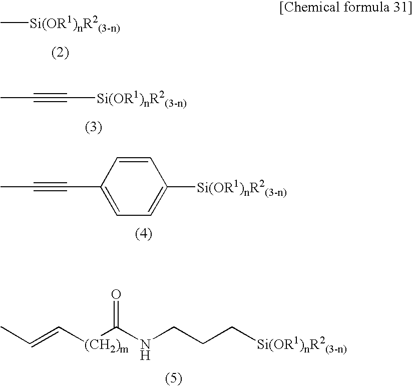
(in the formulae (2) to (5), R¹ represents any one of alkyl groups having 1 to 5 carbon atoms, R² represents an allyl group, n represents an integer in a range from 0 to 3, and m represents an integer in a range from 0 to 6); in the formula (40), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (41), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms.

9. The bridged organosilane according to claim 1, which is a quinacridone-silane compound expressed by the following general formula (42): 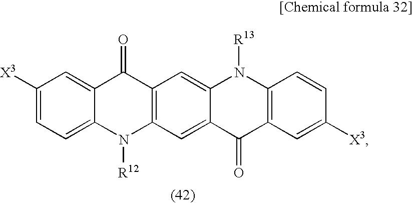
wherein, in the formula (42),
X³— represents a substituent expressed by the following general formula (2):
[Chemical formula 33]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3; and R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms.

10. The bridged organosilane according to claim 1, which is a rubrene-silane compound expressed by any one of the following general formulae (43) and (44): 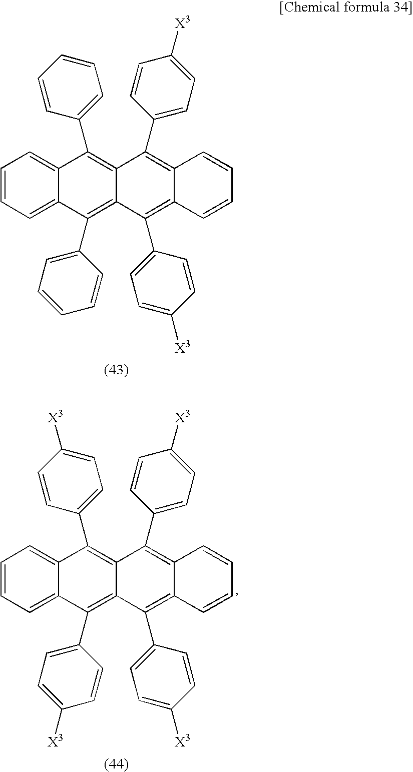
wherein, in the formulae (43) and (44), X³— represents a substituent expressed by the following general formula (2):
[Chemical formula 35]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3).

11. The bridged organosilane according to claim 1, which is a 1,4-alkyloxy-2,5-phenylethenylbenzene-silane compound expressed by the following general formula (45): 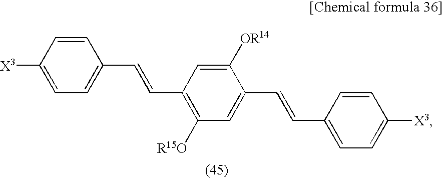
wherein, in the formula (45),
X³— represents a substituent expressed by the following general formula (2):
[Chemical formula 37]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3), and
R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms.

12. The bridged organosilane according to claim 1, which is a triphenylamine-silane compound expressed by the following general formula (46): 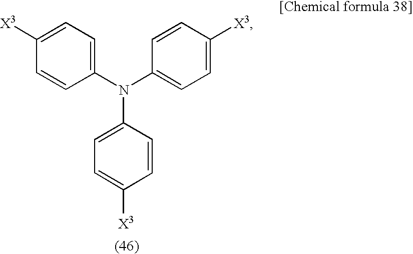
wherein, in the formula (46), X³— represents a substituent expressed by the following general formula (2):
[Chemical formula 39]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3).

13. A bridged organosilane production method, obtaining the bridged organosilane according to claim 1, the method comprising causing a compound expressed by the following general formula (47): 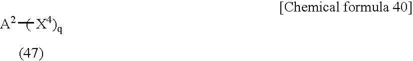
to react with a silane compound expressed by the following general formula (54):
[Chemical formula 55]
H—Si(OR¹)₃ (54)
wherein, in the formula (47),
q represents an integer in a range from 2 to 4,
X⁴— represents a substituent selected from the group consisting of substituents expressed by the following general formulae (48) to (51): 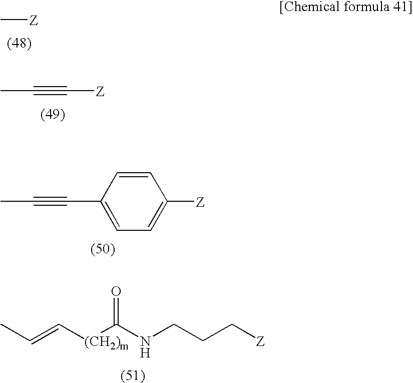
(in the formulae (48) to (51), Z represents any one of a halogen atom, a hydroxy group, and a fluoromethanesulfonate group, m represents an integer in a range from 0 to 6), and
A² represents one organic group selected from the group consisting of organic groups expressed by the following general formula (52): 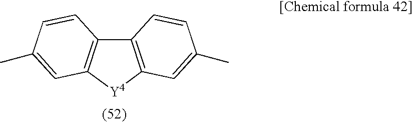
{in the formula (52), Y⁴< represents a substituent selected from the group consisting of substituents expressed by the following general formulae (7) to (11) and (53): 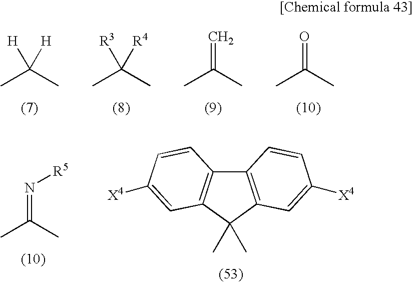
(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (53), X⁴— represents a substituent selected from the group consisting of substituents expressed by the formulae (48) to (51))},
organic groups expressed by the following general formulae (13) and (14): 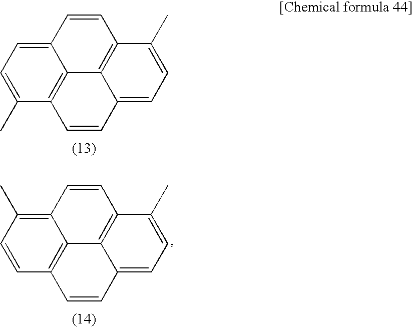
organic groups expressed by the following general formulae (15) to (17): 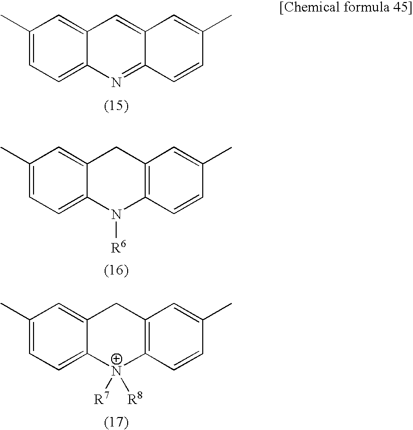
(in the formula (16), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (17), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms),
an organic group expressed by the following general formula (18): 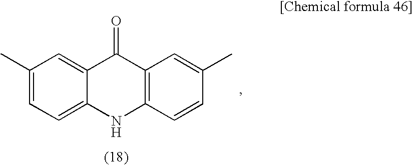
an organic group expressed by the following general formula (19): 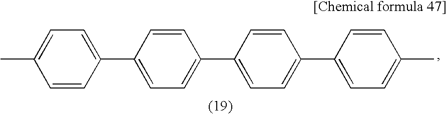
organic groups expressed by the following general formulae (20) and (21): 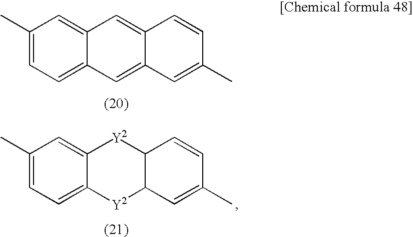
{in the formula (21), Y²< represents a substituent expressed by any one of the following general formulae (10) and (11): 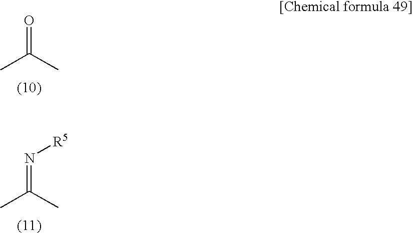
(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms)},
organic groups expressed by the following general formulae (22) and (23): 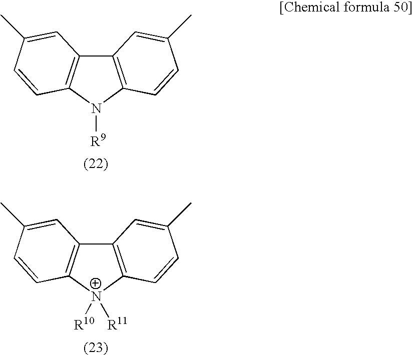
(in the formula (22), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (23), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formula (24): 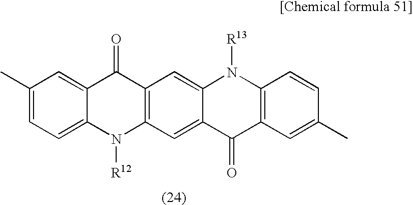
(in the formula (24), R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms),
organic groups expressed by the following general formulae (25) and (26): 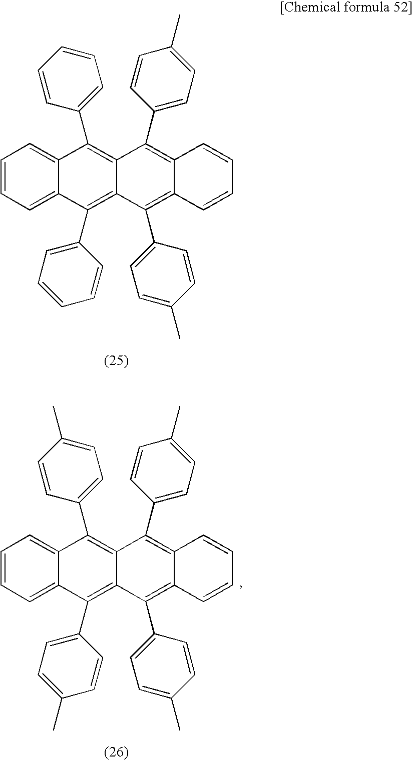
organic groups expressed by the following general formula (27): 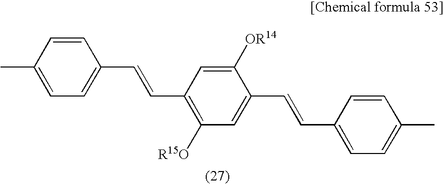
(in the formula (27), R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms), and
an organic group expressed by the following general formula (28): 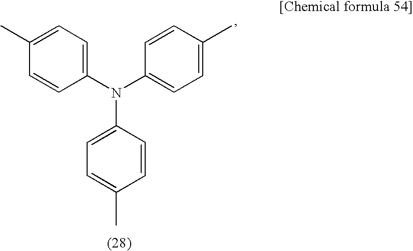
and
wherein, in the formula (54), R¹ represents any one of alkyl groups having 1 to 5 carbon atoms.

14. The bridged organosilane production method according to claim 13, wherein a bridged organosilane which is the fluorene-silane compound expressed by the following general formula (29): 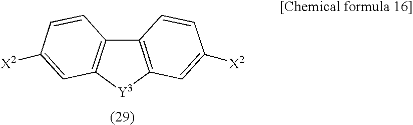
wherein in the formula (29),
X²— represents a substituent selected from the group consisting of substituents expressed by the following general formulae (2) to (4): 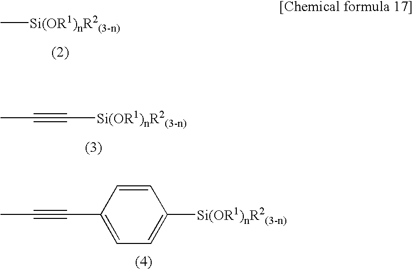
(in the formulae (2) to (4), R¹ represents any one of alkyl groups having 1 to 5 carbon atoms, R² represents an allyl group, and n represents an integer in a range from 0 to 3), and
Y³< represents a substituent selected from the group consisting of substituents expressed by the following general formulae (7) to (11) and (30): 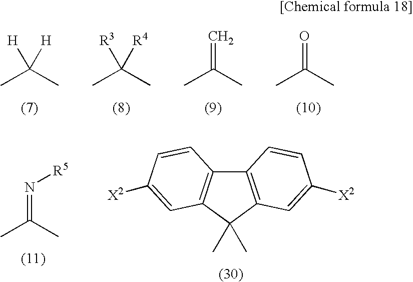
(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (30), X²— represents a substituent selected from the group consisting of substituents expressed by the formulae (2) to (4) is obtained by causing a fluorene compound expressed by the following general formula (55): 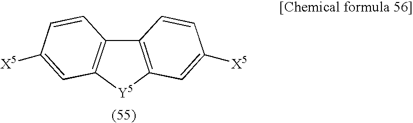
to react with a silane compound expressed by the following general formula (54):
[Chemical formula 59]
H—Si(OR¹)₃ (54)
wherein, in the formula (55),
X⁵— represents a substituent selected from the group consisting of substituents expressed by the following general formulae (48) to (50): 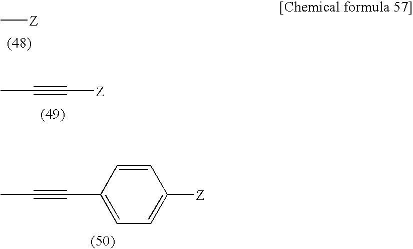
(in the formulae (48) to (50), Z represents any one of a halogen atom, a hydroxy group, and a fluoromethanesulfonate group), and
Y⁵< represents a substituent selected from the group consisting of substituents expressed by the following general formulae (7) to (11) and (56): 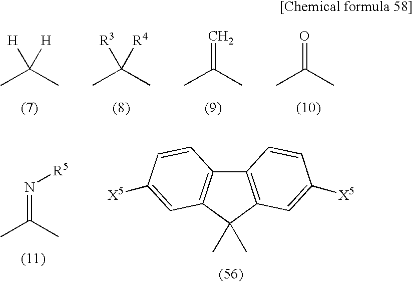
(in the formula (8), R³ and R⁴, which may be the same or different from each other, each represent any one of a hydrogen atom, a hydroxy group, a phenyl group, alkyl groups having 1 to 22 carbon atoms, and perfluoroalkyl groups having 1 to 22 carbon atoms; in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms; and in the formula (56), X⁵— represents a substituent selected from the group consisting of substituents expressed by the formulae (48) to (50)), and wherein,
in the formula (54), R¹ represents any one of alkyl groups having 1 to 5 carbon atoms.

15. The bridged organosilane production method according to claim 13, wherein a bridged organosilane which is the pyrene-silane compound expressed by any one of the following general formulae (31) and (32): 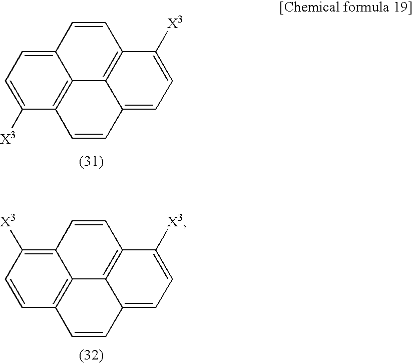
wherein, in the formulae (31) and (32),
X³— represents a substituent expressed by the following general formula (2):
[Chemical formula 20]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3), is obtained by causing a pyrene compound expressed by any one of the following general formulae (57) and (58): 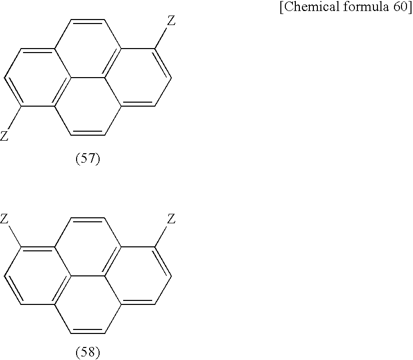
(in the formulae (57) and (58), Z represents any one of a halogen atom, a hydroxy group, and a fluoromethanesulfonate group)
to react with a silane compound expressed by the following general formula (54):
[Chemical formula 61]
H—Si(OR¹)₃ (54)
(in the formula (54), R¹ represents any one of alkyl groups having 1 to 5 carbon atoms).

16. The bridged organosilane production method according to claim 13, wherein a bridged organosilane which is the acridine-silane compound expressed by any one of the following general formulae (33), (34) and (35): 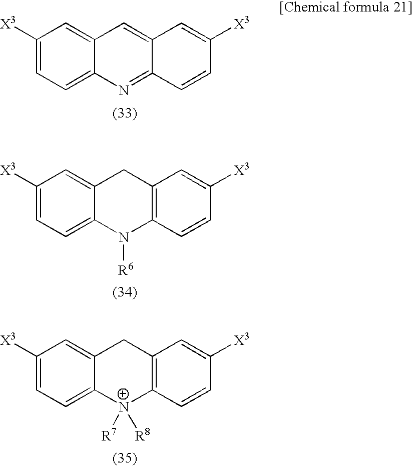
wherein, in the formulae (33) to (35), X³— represents a substituent expressed by the following general formula (2):
[Chemical formula 22]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3); in the formula (34), R⁶ represents any one of a hydrogen atom, alkyl groups having 1 to 22 carbon atoms, perfluoroalkyl groups having 1 to 22 carbon atoms, and aryl groups having 6 to 8 carbon atoms, and; in the formula (35), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms, is obtained by causing an acridine compound expressed by any one of the following general formulae (59), (60) and (61): 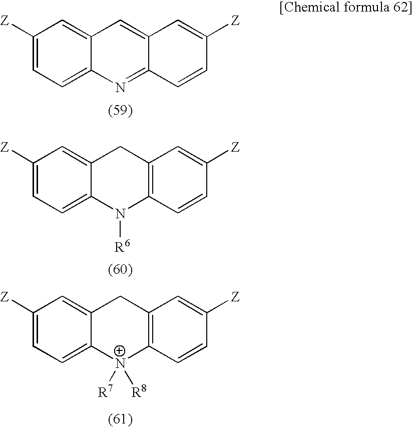
(in the formulae (59) to (61), Z represents any one of a halogen atom, a hydroxy group, and a fluoromethanesulfonate group; in the formula (60), R⁶ represents any one of a hydrogen atom, alkyl groups having 1 to 22 carbon atoms, perfluoroalkyl groups having 1 to 22 carbon atoms, and aryl groups having 6 to 8 carbon atoms; and in the formula (61), R⁷ and R⁸, which may be the same or different from each other, each represent any one of a hydrogen atom, a hydroxy group, a phenyl group, alkyl groups having 1 to 22 carbon atoms, and perfluoroalkyl groups having 1 to 22 carbon atoms)
to react with a silane compound expressed by the following general formula (54):
[Chemical formula 63]
H—Si(OR¹)₃ (54)
(in the formula (54), R¹ represents any one of alkyl groups having 1 to 5 carbon atoms).

17. The bridged organosilane production method according to claim 13, wherein a bridged organosilane which is the acridone-silane compound expressed by the following general formula (36): 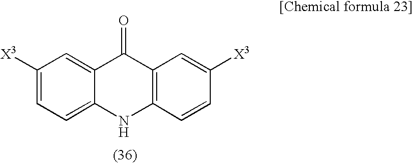
wherein, in the formula (36), X³— represents a substituent expressed by the following general formula (2):
[Chemical formula 24]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3), is obtained by causing an acridone compound expressed by the following general formula (62): 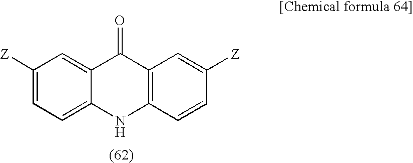
(in the formula (62), Z represents any one of a halogen atom, a hydroxy group, and a fluoromethanesulfonate group)
to react with a silane compound expressed by the following general formula (54):
[Chemical formula 65]
H—Si(OR¹)₃ (54)
(in the formula (54), R¹ represents any one of alkyl groups having 1 to 5 carbon atoms).

18. The bridged organosilane production method according to claim 13, wherein a bridged organosilane which is a quaterphenyl-silane compound expressed by the following general formula (37): 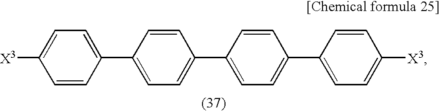
wherein, in the formula (37), X³— represents a substituent expressed by the following general formula (2):
[Chemical formula 26]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3), is obtained by causing a quaterphenyl compound expressed by the following general formula (63): 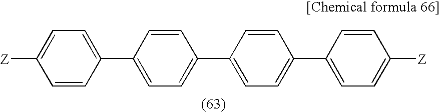
(in the formula (63), Z represents any one of a halogen atom, a hydroxy group, and a fluoromethanesulfonate group)
to react with a silane compound expressed by the following general formula (54):
[Chemical formula 67]
H—Si(OR¹)₃ (54)
(in the formula (54), R¹ represents any one of alkyl groups having 1 to 5 carbon atoms).

19. The bridged organosilane production method according to claim 13, wherein a bridged organosilane which is any one of an anthracene-silane compound, an anthraquinone-silane compound, and an anthraquinonediimine-silane compound, expressed by any one of the following general formulae (38) and (39): 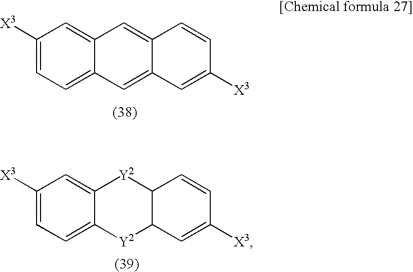
wherein, in the formulae (38) and (39), X³— represents a substituent expressed by the following general formula (2):
[Chemical formula 28]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3; and in the formula (39), Y²< represents a substituent expressed by any one of the following general formulae (10) and (11): 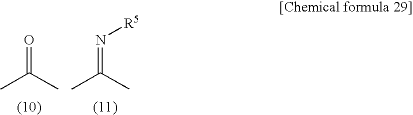
(in the formula (11), R⁵ represents any one of a hydrogen atom, alkyl groups having 1 to 22 carbon atoms, perfluoroalkyl groups having 1 to 22 carbon atoms, and aryl groups having 6 to 8 carbon atoms), is obtained by causing an anthracene compound expressed by the following general formula (64): 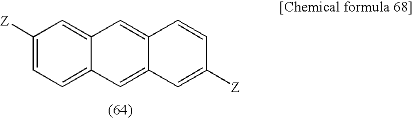
[in the formula (64), Z represents any one of a halogen atom, a hydroxy group, and a fluoromethanesulfonate group]
to react with a silane compound expressed by the following general formula (54):
[Chemical formula 69]
H—Si(OR¹)₃ (54)
(in the formula (54), R¹ represents any one of alkyl groups having 1 to 5 carbon atoms).

20. The bridged organosilane production method according to claim 13, wherein a bridged organosilane which is a carbazole-silane compound expressed by any one of the following general formulae (40) and (41): 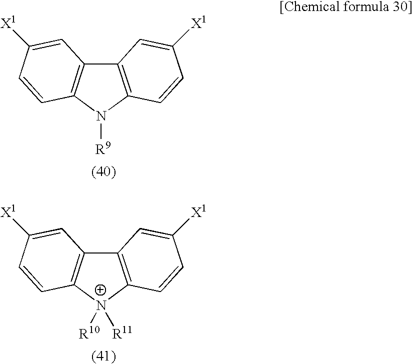
wherein, in the formulae (40) and (41), X¹— represents a substituent selected from the group consisting of substituents expressed by the following general formulae (2) to (5): 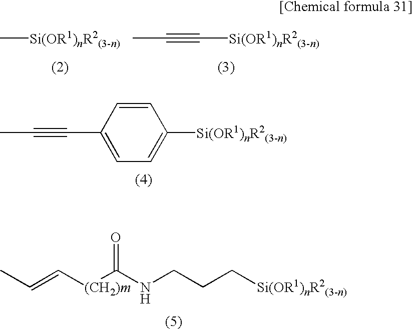
(in the formulae (2) to (5), R¹ represents any one of alkyl groups having 1 to 5 carbon atoms, R² represents an allyl group, n represents an integer in a range from 0 to 3, and m represents an integer in a range from 0 to 6); in the formula (40), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (41), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms is obtained by causing a carbazole compound expressed by any one of the following general formulae (65) and (66): 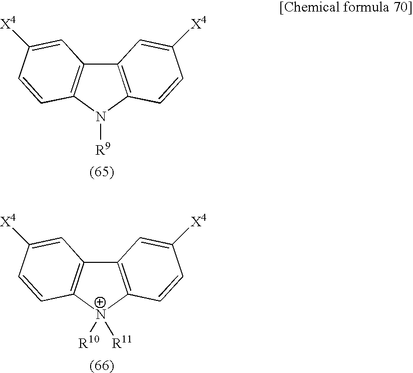
[in the formulae (65) and (66), X⁴— represents a substituent selected from the group consisting of substituents expressed by the following general formulae (48) to (51): 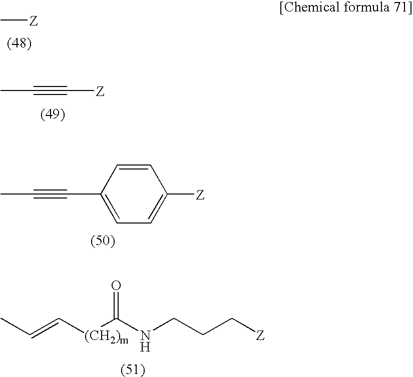
(in the formulae (48) to (51), Z represents any one of a halogen atom, a hydroxy group, and a fluoromethanesulfonate group, and m represents an integer in a range from 0 to 6); in the formula (65), R⁹ represents any one of a hydrogen atom, alkyl groups having 1 to 22 carbon atoms, perfluoroalkyl groups having 1 to 22 carbon atoms, and aryl groups having 6 to 8 carbon atoms; and in the formula (66), R¹⁰ and R¹¹, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms]
to react with a silane compound expressed by the following general formula (54):
[Chemical formula 72]
H—Si(OR¹)₃ (54)
(in the formula (54), R¹ represents any one of alkyl groups having 1 to 5 carbon atoms).

21. The bridged organosilane production method according to claim 13, wherein a bridged organosilane which is a quinacridone-silane compound expressed by the following general formula (42): 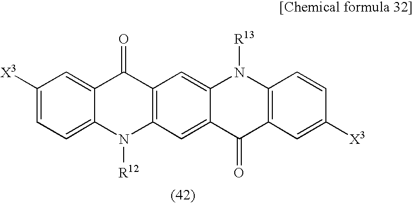
wherein, in the formula (42),
X³— represents a substituent expressed by the following general formula (2):
[Chemical formula 33]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3; and R¹² and R¹³, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms is obtained by causing a quinacridone compound expressed by the following general formula (67): 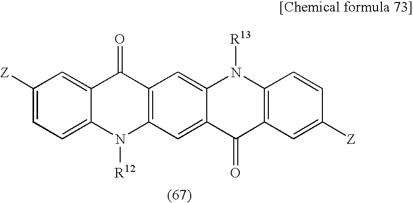
[in the formula (67), Z represents any one of a halogen atom, a hydroxy group, and a fluoromethanesulfonate group]
to react with a silane compound expressed by the following general formula (54):
[Chemical formula 74]
H—Si(OR¹)₃ (54)
(in the formula (54), R¹ represents any one of alkyl groups having 1 to 5 carbon atoms).

22. The bridged organosilane production method according to claim 13, wherein a bridged organosilane which is a rubrene-silane compound expressed by any one of the following general formulae (43) and (44): 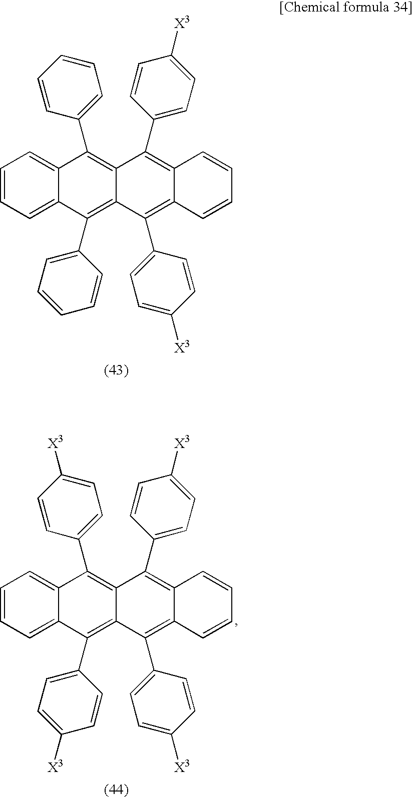
wherein, in the formulae (43) and (44), X³— represents a substituent expressed by the following general formula (2):
[Chemical formula 35]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3) is obtained by causing a rubrene compound expressed by any one of the following general formulae (68) and (69): 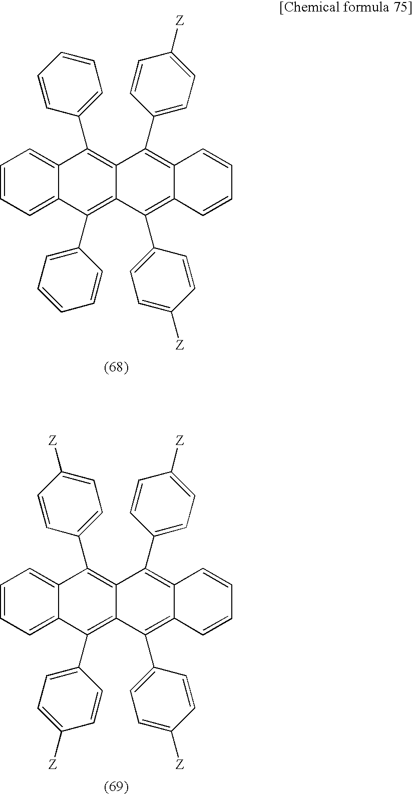
[in the formulae (68) and (69), Z represents any one of a halogen atom, a hydroxy group, and a fluoromethanesulfonate group]
to react with a silane compound expressed by the following general formula (54):
[Chemical formula 76]
H—Si(OR¹)₃ (54)
(in the formula (54), R¹ represents any one of alkyl groups having 1 to 5 carbon atoms).

23. The bridged organosilane production method according to claim 13, wherein a bridged organosilane which is a 1,4-alkyloxy-2,5-phenylethenylbenzene-silane compound expressed by the following general formula (45): 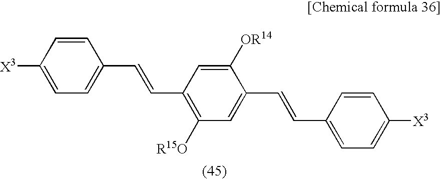
wherein, in the formula (45),
X³— represents a substituent expressed by the following general formula (2):
[Chemical formula 37]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3), and
R¹⁴ and R¹⁵, which may be the same or different from each other, each represent any one of a hydrogen atom, alkyl groups having 1 to 22 carbon atoms, perfluoroalkyl groups having 1 to 22 carbon atoms, and aryl groups having 6 to 8 carbon atoms is obtained by causing a 1,4-alkyloxy-2,5-phenylethenylbenzene compound expressed by the following general formula (70): 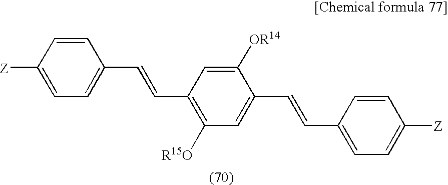
[in the formula (70), Z represents any one of a halogen atom, a hydroxy group, and a fluoromethanesulfonate group]
to react with a silane compound expressed by the following general formula (54):
[Chemical formula 78]
H—Si(OR¹)₃ (54)
(in the formula (54), R¹ represents any one of alkyl groups having 1 to 5 carbon atoms).

24. The bridged organosilane production method according to claim 13, wherein a bridged organosilane which is a triphenylamine-silane compound expressed by the following general formula (46): 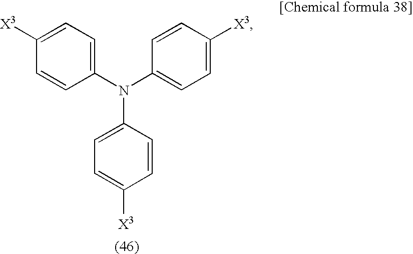
wherein, in the formula (46), X³— represents a substituent expressed by the following general formula (2):
[Chemical formula 39]
—Si(OR¹)ₙR²₍₃₋ₙ₎ (2)
(in the formula (2), R¹ represents any one of alkyl groups having 1 to 5 carbon atoms, R² represents an allyl group, and n represents an integer in a range from 0 to 3) is obtained by causing a triphenylamine compound expressed by the following general formula (71): 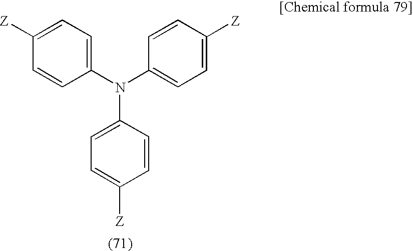
[in the formula (71), Z represents any one of a halogen atom, a hydroxy group, and a fluoromethanesulfonate group]
to react with a silane compound expressed by the following general formula (54):
[Chemical formula 80]
H—Si(OR¹)₃ (54)
(in the formula (54), R¹ represents any one of alkyl groups having 1 to 5 carbon atoms).
